(19) United States
(12) Patent Application Publication
(10) Pub. No.: US 2021/0327534 A1
(43) Pub. Date: Oct. 21, 2021

(54) CANCER CLASSIFICATION USING PATCH CONVOLUTIONAL NEURAL NETWORKS

(71) Applicants: Virgil Nicula, Cupertino, CA (US); Ognjen Nikolic, Menlo Park, CA (US); Yasushi Saito, Mountain View, CA (US); Marius Eriksen, Palo Alto, CA (US); Josh Newman, Mountain View, CA (US); Darya Filippova, Mountain View, CA (US); Alexander Yip, Menlo Park, CA (US); Oliver Venn, San Francisco, CA (US); Joerg Bredno, San Francisco, CA (US); Qinwen Liu, Fremont, CA (US); Alexander P. Fields, Burlingame, CA (US)

(72) Inventors: Virgil Nicula, Cupertino, CA (US); Ognjen Nikolic, Menlo Park, CA (US); Yasushi Saito, Mountain View, CA (US); Marius Eriksen, Palo Alto, CA (US); Josh Newman, Mountain View, CA (US); Darya Filippova, Mountain View, CA (US); Alexander Yip, Menlo Park, CA (US); Oliver Venn, San Francisco, CA (US); Joerg Bredno, San Francisco, CA (US); Qinwen Liu, Fremont, CA (US); Alexander P. Fields, Burlingame, CA (US)

(73) Assignee: GRAIL, INC., Menlo Park, CA (US)

(21) Appl. No.: 17/119,606

(22) Filed: Dec. 11, 2020

Related U.S. Application Data
(60) Provisional application No. 62/948,129, filed on Dec. 13, 2019.

(51) Int. Cl. G16B 20/00; G16B 20/20

(52) U.S. Cl. CPC G16B 20/00; G16B 20/20

(57) ABSTRACT

Methods for determining a disease condition of a subject of a species are provided that comprises obtaining a dataset of fragment methylation patterns determined by methylation sequencing of nucleic acid from a biological sample of the subject. A fragment methylation pattern comprises the methylation state of each CpG site in the fragment. A patch including a channel comprising parameters for the methylation status of respective CpG sites in a set of CpG sites in a reference genome represented by the patch is constructed by populating, for each respective fragment in the plurality of fragments that aligns to the set of CpG sites, an instance of all or a portion of the plurality of parameters based on the methylation pattern of the respective fragment. Application of the patch to a patch convolutional neural network determines the disease condition of the subject.

### CROSS REFERENCE TO RELATED APPLICATION

This application claims priority to U.S. Provisional Patent Application No. 62/948,129 entitled “Cancer Classification Using Patch Convolutional Neural Networks,” filed Dec. 13, 2019, which is hereby incorporated by reference.

### TECHNICAL FIELD

Patch convolutional neural networks that classify subjects for a disease condition, such as cancer, using genotypic information from such subjects are provided.

### BACKGROUND

Earlier detection of cancer is one of the most humane ways to improve cancer outcomes. Status quo treatments—the combination of surgery, chemotherapy and radiation for solid tumors, or chemo and bone marrow transplants for liquid ones—have drawbacks including unsatisfactory survival rates. Treatments often leave patients in pain, while providing an unsatisfactory amount of survival time. New immunotherapies also have drawbacks. Patients have to be treated in intensive care units, and there are often deadly side effects. All such treatments are more effective when cancer is detected early.

However, current screening tests are unsatisfactory. Monitoring methods such as mammography, colonoscopy, Pap smears and testing for prostate specific antigen (PSA) have been in use for decades, but not all are uniformly successful. Some lesions progress so slowly that a patient is more likely to die of something else, while some dangerous tumors still are not detectable before it is too late to cure them. Moreover, to date, no satisfactory screening test is available for lung cancer, among others.

The present disclosure is directed to addressing one or more of these above-referenced challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

The present disclosure addresses the above-identified problems in the art by providing tools for early detection of cancer in subjects. As discussed above, early cancer detection is important because it allows for earlier treatment and therefore a greater chance for survival. Towards that end, the present disclosure provides systems and methods for analyzing methylation states of CpG sites of cfDNA fragments. Sequencing of cell-free DNA (cfDNA) fragments and analysis of methylation states of various dinucleotides of cytosine and guanine (known as CpG sites) in the fragments can provide insight into whether a subject has cancer.

The present disclosure can provide improved specificity and sensitivity over existing classification techniques by applying deep learning classification techniques to methylation fragment data, specifically vision classification techniques. For example, re-framing cancer/non-cancer and tissue-of-origin methylation fragment classifications as a deep learning problem analogous to a vision problem can provide key information on non-linearities in the data such as granular methylation sequence features and higher-order, cross-region features.

The disclosed systems and methods can apply a custom-trained Patch Convolutional Neural Network (Patch-CNN) to the cancer/non-cancer and tissue-of-origin classifications over fragment data from data files. To provide the network with both fine-grained fragment sequence data and visibility into regional locality information, the data can be encoded and represented as a two dimensional “image” with CpG sites along a first axis and depth of piled-up fragment reads along an orthogonal axis and supplemental data encoded as additional channels. CNN architecture can be used in the field of vision and image processing, with the ability to learn common patterns and features across broad sections of data. In the disclosed systems and methods, the positional context of neighboring CpG sites can be encoded and represented similar to image pixels, which are used as inputs for model learning to recognize anomalous sequences and fragments. Similarly, providing a larger region view in terms of the width of CpG sites and depth of reads can provide the network with the ability to learn higher order features across co-localized anomalous fragments.

A major area of concern can include the size of the input features. As such, dimensionality reduction strategies can be employed to make network training feasible. A common obstacle that arises during deep learning applications can include the difficulty of preserving as much information as possible in the underlying data (e.g., at both the fragment level and across regions) while making the problem computationally tractable. For example, a prediction model including every CpG site in the genome or in a targeted methylation panel can contain ˜28M or 1M CpG sites, respectively. Using read depths of ˜30-1500, the network input quickly can rise to more than one billion parameters. The network size, depth, computational complexity, memory constraints and imbalance of number of training examples compared to input parameters can be simply intractable, particularly for traditional deep learning databases and large image classifiers that operate on a maximum of 28×28 images or thirty to fifty thousand inputs. While there are dimensionality reductions that pre-filter, aggregate and bin data into coarser resolution, they can reduce information available for classification.

One option for dimensionality reduction can include subdividing the input space into more tractable, localized regions that can be learned independently before merging. This can be equivalent to conducting localized, sharded searches that attempt to explore regions independently before merging results. Thus, as is described herein in the present disclosure, a genome or panel of CpG sites can be represented as a large image segmented into manageable regions for use in Patch-CNN, transforming disease prediction into a more tractable problem. The present disclosure can further provide systems and methods for the framing and structuring of fragment data into data constructs, such as matrices, for stable and reproducible classification.

Thus, the present disclosure can provide systems and methods for improving performance gains for fragment, region, and sample-level classification using deep neural nets (e.g., Patch-CNN) on methylation sequencing data. Furthermore, the present disclosure can provide systems and methods for improving assessment of features at granularities other than anomalous methylation states, including fine granularity methylation sequence features and coarse granularity cross-region patterns. Such applications can improve the sensitivity and specificity of performance of predictions (e.g., cancer/non-cancer and tissue-of-origin) while also identifying the CpG regions of interest that provide the most information gain compared to conventional analysis workflows.

Thus, the present disclosure can provide methods for determining a disease condition of a test subject of a species. In one such aspect of the present disclosure, the method is performed at a computer system including at least one processor and a memory storing at least one program for execution by the at least one processor. The at least one program can include instructions for obtaining a dataset, in electronic form, where the dataset comprises a corresponding methylation pattern of each respective fragment in a plurality of fragments. The corresponding methylation pattern of each respective fragment can be determined by a methylation sequencing of one or more nucleic acid samples including the respective fragment in a biological sample obtained from the test subject and includes a methylation state of each CpG site in a corresponding plurality of CpG sites in the respective fragment.

In this aspect, the at least one program further includes instructions for constructing a first patch including a first channel. The first patch can represent a first independent set of CpG sites in a reference genome of the species, and each respective CpG site in the first independent set of CpG sites corresponds to a predetermined location in the reference genome. The first channel of the first patch can include a plurality of instances of a first plurality of parameters. Each instance of the first plurality of parameters can include a parameter for a methylation status of a respective CpG site in the first independent set of CpG sites for the first patch. Construction of the first patch can comprise populating, for each respective fragment in the plurality of fragments that aligns to the first independent set of CpG sites, an instance of all or a portion of the first plurality of parameters based on the methylation pattern of the respective fragment.

In this aspect, the at least one program can further include instructions for applying at least the first patch to a classifier thereby determining the cancer condition in the test subject.

In some embodiments, the at least one program further comprises instructions for, after obtaining the dataset and prior to constructing the first patch, pruning the plurality of fragments. The plurality of fragments can be pruned by removing from the plurality of fragments each respective fragment, whose corresponding methylation pattern across a corresponding plurality of CpG sites in the respective fragment has a p-value that fails to satisfy a p-value threshold. The p-value of the respective fragment can be determined based upon a comparison of the corresponding methylation pattern of the respective fragment to a corresponding distribution of methylation patterns of the corresponding plurality of CpG sites in a corresponding plurality of reference fragments that have the corresponding plurality of CpG sites of the respective fragment. The methylation pattern of each reference fragment in the corresponding plurality of reference fragments can be obtained by a methylation sequencing of nucleic acid from biological samples obtained from a cohort of subjects that have one or more common characteristics (e.g., a cohort of healthy subjects, a cohort of healthy subjects that smoke, a cohort of subjects that do not smoke, a cohort of male subjects, a cohort of female subjects, a cohort of subjects that are above a threshold age, a cohort of subjects that are in a specified age range, a cohort of subjects that have a particular set of genetic mutations, a cohort of subjects of a particular race, etc.).

In some embodiments, the first patch comprises a plurality of channels including the first channel and a second channel. The second channel can comprise a corresponding instance of a second plurality of parameters for each instance of the first plurality of parameters. Each instance of the second plurality of parameters can include a parameter for a first characteristic, other than CpG methylation state, of a respective CpG site in the first independent set of CpG sites for the first patch. Constructing the first patch can comprise populating, for each respective fragment in the plurality of fragments that aligns to the first independent set of CpG sites, an instance of all or a portion of the first plurality of parameters and an instance of all or a portion of the second plurality of parameters based on the methylation pattern of the respective fragment.

In some embodiments, the methylation pattern of a respective fragment does not include each CpG site in the first independent set of CpG sites of the first patch. Constructing the first patch, for a respective fragment in the plurality of fragments, can comprise populating parameters in the instance of first plurality of parameters that correspond to CpG sites present in the respective fragment.

In some embodiments, constructing the first patch, for a respective fragment in the plurality of fragments, comprises identifying, within an instance of the first plurality of parameters of the first channel, parameters corresponding to the CpG sites in the respective fragment that have not previously been assigned methylation states based on another fragment in the plurality of fragments. Constructing the first patch can further comprise assigning, for each parameter among the identified parameters that aligns to a corresponding CpG site of the respective fragment, the methylation state of the corresponding CpG site of the respective fragment.

In some embodiments, for a respective fragment in the plurality of fragments, constructing the first patch comprises identifying, within an instance of the first plurality of parameters of the first channel, parameters corresponding to the CpG sites in the respective fragment that have not previously been assigned methylation states based on another fragment in the plurality of fragments. Constructing the first patch can further comprise assigning, for each parameter among the identified parameters that aligns to a respective CpG site of the respective fragment, the methylation state of the respective CpG site of the respective fragment. Constructing the first patch can further comprise assigning, for each parameter among the identified parameters, in the second plurality of parameters of the instance of the second plurality of parameters of the second channel that corresponds to the instance of the first plurality of parameters, that aligns to a respective CpG site of the respective fragment, the first characteristic of the respective CpG site of the respective fragment. In some embodiments, the first characteristic of the respective CpG site is a multiplicity of the respective fragment the respective CpG site is on. In some embodiments, the first characteristic of the respective CpG site comprises a CpG β-value drawn from a cohort of subjects that have one or more common characteristics described elsewhere herein, a CpG β-value drawn from a predetermined tissue type in a cohort of subjects that have one or more common characteristics described elsewhere herein, a CpG β-value drawn from the test subject, a Pearson's correlation score for methylation state of 5′ and 3′ neighbor CpG sites, a Jaccard similarity, Euclidean distance, Manhattan distance, maximum value, normalized Euclidean distance, normalized maximum value, Dice coefficient, or cosine similarity of methylation state of the respective CpG site in the test subject versus a cancer cohort or a cohort of subjects that have one or more common characteristics described elsewhere herein, a fragment p-value of the respective fragment, a length of the respective fragment the respective CpG site is on, a fragment sequence source, a fragment mapping quality score of the respective fragment the respective CpG site is on, a distance to a 5′ adjacent CpG site in the reference genome, a distance to a 3′ adjacent CpG site in the reference genome, a multiplicity of the respective fragment the respective CpG site is on, a genetic element the respective CpG site is within, a biological pathway the respective CpG site is associated with, a gene the respective CpG site is associated with, a value of a CpG transition impulse function for the respective CpG site, a value of a CpG run-length encoding for the respective CpG site, and a read strand orientation of the fragment the respective CpG site is on. In some embodiments, more than one fragment in the plurality of fragments is assigned to a single instance of the first plurality of parameters of the first channel in the first patch provided that more than one fragment does not have common CpG sites.

In some embodiments, parameters in the instance of the first plurality of parameters are zero filled. In some embodiments, the first independent set of CpG sites are in a CpG index of the reference genome. In some such embodiments, the CpG index of the reference genome includes a first CpG site, not present in the first independent set of CpG sites, located in the reference genome between a second CpG site and a third CpG site that are present in the first independent set of CpG sites.

In some embodiments, the first independent set of CpG sites includes a first CpG site and a second CpG site that are adjacent to each other in a CpG index of the reference genome. A first fragment in the plurality of fragments can include the first CpG site but not the second CpG site. A second fragment in the plurality of fragments can include the second CpG site but not the first CpG site.

In some embodiments, a parameter in an instance of the first plurality of parameters, for a respective fragment in the plurality of fragments, is: methylated when the corresponding CpG site in the respective fragment is determined by the methylation sequencing to be methylated, unmethylated when the corresponding CpG site in the respective fragment is determined by the methylation sequencing to not be methylated, and/or other when the corresponding CpG site in the respective fragment is determined by the methylation sequencing to be other than methylated or unmethylated.

In some embodiments, a number of instances of the first plurality of parameters of the first channel are not assigned a respective fragment, and the at least one program further comprises instructions for zero filling parameters in instances of the plurality of parameters of the first channel that have not been assigned a fragment. In some embodiments, where the at least one program is unable to identify, within an instance of the first plurality of parameters of the first channel, parameters corresponding to the CpG sites in the respective fragment that have not previously been assigned methylation states based on another fragment in the plurality of fragments, the at least one program further comprises instructions for discarding the respective fragment. In some embodiments, where the at least one program is unable to identify, within an instance of the first plurality of parameters of the first channel of the first patch, parameters corresponding to the CpG sites in the respective fragment that have not previously been assigned methylation states based on another fragment in the plurality of fragments, the at least one program further comprises instructions for creating an additional instance of the first patch and assigning the respective fragment to the additional instance of the first patch.

In some embodiments, the plurality of channels comprises at least three channels. A third channel in the first plurality of channels can comprise a corresponding instance of a third plurality of parameters for each instance of the first plurality of parameters. Each instance of the third plurality of parameters can include a parameter for a second characteristic of a respective CpG site in the first independent set of CpG sites. The second characteristic can comprise a CpG β-value drawn from a cohort of subjects that have one or more common characteristics described elsewhere herein, a CpG β-value drawn from a predetermined tissue type in a cohort of subjects that have one or more common characteristics described elsewhere herein, a CpG β-value drawn from the test subject, a Pearson's correlation score for methylation state of 5′ and 3′ neighbor CpG sites, a Jaccard similarity of methylation state of the respective CpG site in test subject versus a cancer cohort or a cohort of subjects that have one or more common characteristics described elsewhere herein, a fragment p-value of the respective fragment, a length of the respective fragment the respective CpG site is on, a fragment sequence source, a fragment mapping quality score of the respective fragment the respective CpG site is on, a distance to a 5′ adjacent CpG site in the reference genome, a distance to a 3′ adjacent CpG site in the reference genome, a multiplicity of the respective fragment the respective CpG site is on, a genetic element the respective CpG site is within, a biological pathway the respective CpG site is associated with, a gene the respective CpG site is associated with, a value of a CpG transition impulse function for the respective CpG site, a value of a CpG run-length encoding for the respective CpG site, and a read strand orientation of the fragment the respective CpG site is on.

In some embodiments, the first independent set of CpG sites is drawn from across the entire reference genome. In some embodiments, the at least one program further includes instructions for constructing a second patch including a corresponding first channel. The second patch can represent a second independent set of CpG sites in the reference genome of the species. Each respective CpG site in the second independent set of CpG sites can correspond to a predetermined location in the reference genome. The corresponding first channel of the second patch can comprise a corresponding plurality of instances of a first plurality of parameters. Each instance of the corresponding first plurality of parameters of the first channel of the second patch can include a parameter for a methylation status of a respective CpG site in the second independent set of CpG sites for the second patch. The at least one program can further include instructions for populating, for each respective fragment in the plurality of fragments that aligns to the second independent set of CpG sites, an instance of all or a portion of the first plurality of parameters of the second patch based on the methylation pattern of the respective fragment thereby constructing the second patch. The instructions can further comprise applying the first and second patches to the classifier thereby determining the cancer condition in the test subject. In some embodiments, the second patch can comprise a corresponding plurality of channels including the corresponding first channel. A corresponding second channel in the corresponding plurality of channels of the second patch can comprise a corresponding instance of a second plurality of parameters for each instance of the first plurality of parameters. Each instance of the second plurality of parameters of the second patch can include a parameter for a first characteristic, other than CpG methylation state, of a respective CpG site in the second independent set of CpG sites for the second patch. The instructions for populating, for each respective fragment in the plurality of fragments that aligns to the second independent set of CpG sites, can further populate an instance of all or a portion of the instance of the second plurality of parameters of the second patch based on the methylation pattern of the respective fragment.

In some embodiments, the first independent set of CpG sites does not overlap with the second independent set of CpG sites. In some other such embodiments, the first independent set of CpG sites overlaps with the second independent set of CpG sites. In some embodiments, the first patch represents an equally sized, but different, portion of the reference genome than the second patch. In some other such embodiments, the first patch represents a first portion of the reference genome and the second patch represents a second portion of the reference genome, where a size of the first portion is different than a size of the second portion. In some embodiments, the first independent set of CpG sites comprises a first number of CpG sites, the second independent set of CpG sites comprises a second number of CpG sites, and the first number of CpG sites is the same as the second number of CpG sites. In some other such embodiments, the first independent set of CpG sites comprises a first number of CpG sites, the second independent set of CpG sites comprises a second number of CpG sites, and the first number of CpG sites is different than the second number of CpG sites.

In some embodiments, the methylation sequencing of one or more nucleic acid samples is whole genome methylation sequencing or targeted DNA methylation sequencing using a plurality of nucleic acid probes. In some such embodiments, the methylation sequencing of one or more nucleic acid samples uses a plurality of nucleic acid probes. In some embodiments, the methylation sequencing of one or more nucleic acid samples detects one or more 5-methylcytosine (5mC) and/or 5-hydroxymethylcytosine (5hmC) in the respective fragment. As disclosed herein, the term “methylation” analysis can cover any type of modification involving a methyl group, including but not limited to hydroxymethylation.

In some embodiments, the methylation sequencing of one or more nucleic acid samples comprises conversion of one or more unmethylated cytosines or one or more methylated cytosines, in the respective fragment, to a corresponding one or more uracils. In some embodiments, the one or more uracils are detected during the methylation sequencing as one or more corresponding thymines. In some other such embodiments, the conversion of one or more unmethylated cytosines or one or more methylated cytosines comprises a chemical conversion, an enzymatic conversion, or combinations thereof.

In some embodiments, the at least one program further comprises instructions for constructing a plurality of patches including the first patch, each respective patch being for a different independent set of CpG sites in the reference genome. Constructing the first patch can further comprise constructing a plurality of patches including the first patch. The classifier can comprise one or more trained first stage models (e.g., a single first stage model for all patches or a plurality of trained first stage models each corresponding to a patch) and a second stage model. The applying the at least the first patch to a classifier can comprise obtaining a feature vector comprising a plurality of feature elements. Each feature element in the plurality of feature elements can be an output of a corresponding trained first stage model in the plurality of trained first stage models upon application of a respective patch in the plurality of patches to the corresponding trained first stage model. The instructions can further include applying the feature vector to the second stage model thereby determining the cancer condition in the test subject. In some embodiments, each respective trained first stage model in the plurality of trained first stage models is a corresponding trained convolutional neural network and the second stage model is a logistic regression model. In some embodiments, the second stage model can be a binary classification algorithm or a multinomial classification algorithm (e.g., for classifying tissue of origin). In some embodiments, the second stage classification algorithm can be based on a GradientBoosting algorithm, a decision tree algorithm, a random forest algorithm, a K nearest neighbors algorithm, a Gaussian NB algorithm, a deep neural Network algorithm, or any combinations thereof.

The first channel of the first patch can be two dimensional with each respective instance of the plurality of instances of the first plurality of parameters of the first patch forming a first dimension and the first plurality of parameters of the first patch forming the second dimension. In some embodiments, the plurality of patches is between 10 patches and 10000 patches. In some other such embodiments, the plurality of patches is between 100 patches and 3000 patches.

In some embodiments, the classifier comprises a plurality of first stage models and a dynamic neural network. The at least one program can further include instructions for constructing a plurality of patches including the first patch, each respective patch being for a different set of CpG sites in the reference genome. Constructing the plurality of patches can construct a respective patch including the first patch. Applying the at least the first patch to a classifier can comprise applying each respective patch in the plurality of patches to a corresponding first stage model in the plurality of first stage model. The corresponding first stage model can comprise a respective input layer for receiving the respective patch, where the respective patch comprises a first number of dimensions. The corresponding first stage model can further comprise a respective fully connected embedding layer that comprises a corresponding set of weights. The respective fully connected embedding layer can directly or indirectly receive output of the respective input layer. A respective output of the respective embedding layer can be a second number of dimensions that is less than the first number of dimensions. The corresponding first stage model can further comprise a respective output layer that directly or indirectly receives output from the respective fully connected embedding layer. Applying the at least the first patch to a classifier can further comprise inputting an aggregate of the respective output from each respective fully connected embedding layer of each trained first stage model in the plurality of first stage models into the dynamic neural network thereby determining the cancer condition in the test subject. In some such embodiments, the respective output of the respective embedding layer of each respective first stage model in the plurality of first stage models can include a set of between 32 and 1048 values. In some further such embodiments, the at least one program further comprises instructions for training the plurality of first stage models and the dynamic neural network using a cohort of subjects. In some such embodiments, the cohort of subjects comprises a first subset of subjects that have a first label for the cancer condition and a second subset of subjects that have a second label for the cancer condition. In some embodiments, a single first stage model is trained on multiple patches per sample across a group of samples (e.g., the samples are obtained from a group of training subjects having a known cancer status).

The trained first stage model can then be applied to sequencing data from a test sample from a subject of unknown status to extract feature elements from each patch. For example, the sequencing data can be processed according to the same set of patches used for training (e.g., Patch 530-1, Patch 530-2, all through Patch 530-K). The single first stage model can then be applied to each patch (e.g., Trained Model 1, Trained Model 2, . . . , and Trained Model K of FIG. 7A are in fact the same trained model), using sequencing data from the group of training subjects, to separately extract features and/or feature elements from each respective patch (e.g., Feature element 1, feature element 2, . . . and feature element K). In some embodiments, a mixed approach can be taken. In particular, a plurality of first stage models can be trained and used to obtain features and/or feature elements for further sample-level classification. For example, multiple patches can be used to train a common first stage model per sample across a group of samples (e.g., the samples are obtained from a group of training subjects having a known cancer status). The same common first stage model can be applied to corresponding patches based on sequencing data of a sample from a subject to extract features and/or feature elements from the subject. In other embodiments, a single first stage model is trained with a single patch per sample across a group of samples (e.g., the samples are obtained from a group of training subjects having a known cancer status). For example, if the dataset has 10000 samples, the models trained on single patch per sample can be trained 10000 times. The particular first stage model can then be applied to a corresponding patch from the subject to extract features and/or feature elements from the subject. The features and/or feature elements from all patches being examined for this particular subject can then be used to perform a sample level classification. For example, as illustrated in FIG. 7A, Trained Model 1 and Trained Model 2 of FIG. 7A can be the same while Trained Model K can be specific for Patch 530-K). The shared model can be used to extract feature elements from Patches 530-1 and 530-2 while the individualized model is used to extract feature element(s) from Patch 530-K. Regardless of the number of first stage models that are trained, the same number of feature elements can be presented to the sample level classifier for classification.

In some further such embodiments, the instructions for training comprise stratifying, on a random basis, the cohort of subjects into a plurality of groups based on any combination of cancer condition, age, smoking status, or sex. The instructions for training can further comprise using a first group in the plurality of groups as a training group and the remainder of the plurality of groups as test groups to train the plurality of models and the dynamic neural network against the training group. The instructions for training can further comprise repeating using the groups for training and test groups, for each group in the plurality of groups, so that each group in the plurality of groups is used as the training group in an iteration. The instructions for training can further comprise repeating the stratifying, using groups and repeating iterations until a classifier performance criterion is satisfied. In some further such embodiments, the cancer condition is tissue of origin and each subject in the cohort of subjects is labeled with a tissue of origin. In some further such embodiments, the cohort includes subjects that have an anorectal cancer, a bladder cancer, a breast cancer, a cervical cancer, a colorectal cancer, a head and neck cancer, a hepatobiliary cancer, an endometrial cancer, a kidney cancer, a leukemia, a liver cancer, a lung cancer, a lymphoid neoplasm, a melanoma, a multiple myeloma, a myeloid neoplasm, an ovary cancer, a non-Hodgkin lymphoma, a pancreatic cancer, a prostate cancer, a renal cancer, a thyroid cancer, an upper gastrointestinal tract cancer, a urothelial carcinoma, or a uterine cancer.

In some further such embodiments, the cancer condition is a stage of an anorectal cancer, a stage of bladder cancer, a stage of breast cancer, a stage of cervical cancer, a stage of colorectal cancer, a stage of head and neck cancer, a stage of hepatobiliary cancer, a stage of endometrial cancer, a stage of kidney cancer, a stage of leukemia, a stage of liver cancer, a stage of lung cancer, a stage of lymphoid neoplasm, a stage of melanoma, a stage of multiple myeloma, a stage of myeloid neoplasm, a stage of ovary cancer, a stage of non-Hodgkin lymphoma, a stage of pancreatic cancer, a stage of prostate cancer, a stage of renal cancer, a stage of thyroid cancer, a stage of upper gastrointestinal tract cancer, a stage of urothelial carcinoma, or a stage of uterine cancer. In some such embodiments, the cancer condition is whether or not a subject has cancer and the stratifying the cohort of subjects ensures that each group in the plurality groups has equal numbers of subjects that have cancer and that do not have cancer.

In some such embodiments, the training eliminates one or more patches in the plurality of patches using L1 or L2 regularization based upon values provided by the respective output layer of each respective patch in the plurality of patches during the training. In some embodiments, the plurality of instances of the first plurality of parameters is between 24 and 2048. In some embodiments, a number of instances in the plurality of instances of the first plurality of parameters is determined based on expected read depth of the plurality of fragments plus one standard deviation across the plurality of fragments. In some embodiments, the constructing patches further comprises sorting respective fragments assigned to the first patch based on their respective p-values or their starting position in the reference genome.

In some embodiments, the at least one program further comprises instructions for selecting the first independent set of CpG sites of the first patch through evaluation of a plurality of CpG methylation patterns. The plurality of CpG methylation patterns can be determined by a methylation sequencing of a plurality of clinical fragments obtained from a plurality of clinical nucleic acid samples of a plurality of clinical biological samples obtained from a clinical cohort comprising a plurality of clinical subjects. The plurality of clinical subjects can include a first set of clinical subjects that have a first indication for the cancer condition and a second set of clinical subjects that have a second indication for the cancer condition.

In some such embodiments, the instructions for selecting a set of CpG sites comprise determining a first ranking of a plurality of CpG sites in the reference genome based upon a respective first mutual information score for a methylation status of each CpG site in the plurality of CpG sites between the first set of clinical subjects and the second set of clinical subjects. The instructions can further comprise selecting a first threshold number of CpG sites for the corresponding independent set of CpG sites for the first patch using the ranking. In some further such embodiments, the plurality of clinical subjects includes a third set of clinical subjects that have a third indication for the cancer condition and a fourth set of clinical subjects that have a fourth indication for the cancer condition. In some such embodiments, the instructions for selecting further comprise determining a second ranking of the plurality of CpG sites in the reference genome based upon a respective second mutual information score for a methylation status of each CpG site in the plurality of CpG sites between the third set of clinical subjects and the fourth set of clinical subjects. The instructions can further comprise selecting a second threshold number of CpG sites for the first independent set of CpG sites of the first patch using the second ranking. In some such embodiments, constructing patches further comprises sorting respective fragments assigned to the first patch based on their respective first or second mutual information score. In some such embodiments, the first indication for the cancer condition is a first cancer type and the second indication for the cancer condition is a second cancer type. In some such embodiments, each respective CpG site in the first threshold number of CpG sites for the first independent set of CpG sites of the first patch is padded in the reference genome from all other CpG sites in the first threshold number of CpG sites by a threshold number of residues.

In some such embodiments, the instructions for selecting a set of CpG sites further comprise determining a first ranking of a plurality of fixed length regions in the reference genome based upon a respective first mutual information score for a methylation status of a CpG site methylation pattern of each fixed length region in the plurality of fixed length regions between the first set of clinical subjects and the second set of clinical subjects. The instructions for selecting can further comprise selecting a first threshold number of CpG sites for the first independent set of CpG sites of the first patch from those fixed length regions in the plurality of fixed length regions using the first ranking. In some further such embodiments, the plurality of clinical subjects includes a third set of clinical subjects that have a third indication for the cancer condition and a fourth set of clinical subjects that have a fourth indication for the cancer condition. The instructions for selecting can further comprise determining a second ranking of the plurality of fixed length regions in the reference genome based upon a respective second mutual information score for a methylation status of a CpG site methylation pattern of each fixed length region in the plurality of fixed length regions between the third set of clinical subjects and the fourth set of clinical subjects. The instructions for selecting can further comprise selecting a second threshold number of CpG sites for the first independent set of CpG sites of the first patch using the second ranking. In some such embodiments, constructing patches further comprises sorting respective fragments assigned to the first patch based on their respective first or second mutual information score. In some embodiments, the one or more nucleic acid samples are cell-free nucleic acid samples.

Another aspect of the present disclosure provides a computer system for determining a cancer condition of a test subject of a species. Any methods disclosed herein can also be used to determine disease conditions (e.g., genetic disorders) other than cancer conditions. In this aspect, the computer system comprises at least one processor and a memory storing at least one program for execution by the at least one processor. The at least one program can comprise instructions for obtaining a dataset in electronic form. The dataset can comprise a corresponding methylation pattern of each respective fragment in a plurality of fragments. The corresponding methylation pattern of each respective fragment can be determined by a methylation sequencing of one or more nucleic acid samples comprising the respective fragment in a biological sample obtained from the test subject and comprises a methylation state of each CpG site in a corresponding plurality of CpG sites in the respective fragment. In this aspect, the at least one program further includes instructions for constructing a first patch comprising a first channel. The first patch can represent a first independent set of CpG sites in a reference genome of the species. Each respective CpG site in the first independent set of CpG sites can correspond to a predetermined location in the reference genome. The first channel of the first patch can comprise a plurality of instances of a first plurality of parameters, and each instance of the first plurality of parameters includes a parameter for a methylation status of a respective CpG site in the first independent set of CpG sites for the first patch. Constructing the first patch can comprise populating, for each respective fragment in the plurality of fragments that aligns to the first independent set of CpG sites, an instance of all or a portion of the first plurality of parameters based on the methylation pattern of the respective fragment. In this aspect, the at least one program further includes instructions for applying at least the first patch to a classifier thereby determining the cancer condition in the test subject.

Another aspect of the present disclosure provides a non-transitory computer-readable storage medium storing program code instructions that, when executed by a processor, cause the processor to perform a method of determining a cancer condition of a test subject of a species. The method can include obtaining a dataset in electronic form. The dataset can comprise a corresponding methylation pattern of each respective fragment in a plurality of fragments. The corresponding methylation pattern of each respective fragment can be determined by a methylation sequencing of one or more nucleic acid samples comprising the respective fragment in a biological sample obtained from the test subject and comprises a methylation state of each CpG site in a corresponding plurality of CpG sites in the respective fragment. In this aspect, the method further includes constructing a first patch comprising a first channel. The first patch can represent a first independent set of CpG sites in a reference genome of the species. Each respective CpG site in the first independent set of CpG sites can correspond to a predetermined location in the reference genome. The first channel of the first patch can comprise a plurality of instances of a first plurality of parameters, and each instance of the first plurality of parameters includes a parameter for a methylation status of a respective CpG site in the first independent set of CpG sites for the first patch. Constructing the first patch can comprise populating, for each respective fragment in the plurality of fragments that aligns to the first independent set of CpG sites, an instance of all or a portion of the first plurality of parameters based on the methylation pattern of the respective fragment. In this aspect, the method further comprises applying at least the first patch to a classifier thereby determining the cancer condition in the test subject.

Another aspect of the present disclosure provides a method of determining a cancer condition of a test subject of a species. In this aspect, the method is provided at a computer system comprising at least one processor and a memory storing at least one program for execution by the at least one processor. The at least one program can comprise instructions for obtaining a dataset in electronic form, where the dataset comprises a corresponding methylation pattern of each respective fragment in a plurality of fragments. The corresponding methylation pattern of each respective fragment can be determined by a methylation sequencing of one or more nucleic acid samples of the respective fragment in a biological sample obtained from the test subject and can comprise a methylation state of each CpG site in a corresponding plurality of CpG sites in the respective fragment.

In this aspect, the at least one program further includes instructions for obtaining a plurality of patches, where each respective patch in the plurality of patches comprises a first channel and represents a corresponding independent set of CpG sites in a reference genome of the species. Each respective CpG site in the corresponding independent set of CpG sites can correspond to a predetermined location in the reference genome. The first channel for a respective patch can comprise a plurality of instances of a first plurality of parameters, where each instance of the first plurality of parameters includes a parameter for a methylation status of a respective CpG site in the corresponding independent set of CpG sites for the respective patch.

In this aspect, the at least one program can further include instructions for assigning all or a portion of each respective fragment in the plurality of fragments to a respective patch in the plurality of patches based upon a match between CpG sites of the respective fragment and the corresponding independent set of CpG sites of the single respective patch. In this aspect, the at least one program further includes instructions for applying each respective patch in the plurality of patches to a corresponding trained model in a plurality of models thereby determining the cancer condition in the test subject.

Another aspect of the present disclosure provides a computer system for determining a cancer condition of a test subject of a species that comprises at least one processor and a memory storing at least one program for execution by the at least one processor. The at least one program can comprise instructions for obtaining a dataset, in electronic form, where the dataset comprises a corresponding methylation pattern of each respective fragment in a plurality of fragments. The corresponding methylation pattern of each respective fragment can be determined by a methylation sequencing of one or more nucleic acid samples of the respective fragment in a biological sample obtained from the test subject and can comprise a methylation state of each CpG site in a corresponding plurality of CpG sites in the respective fragment. In this aspect, the at least one program can further comprise instructions for obtaining a plurality of patches, where each respective patch in the plurality of patches comprises a first channel and represents a corresponding independent set of CpG sites in a reference genome of the species. Each respective CpG site in the corresponding independent set of CpG sites can correspond to a predetermined location in the reference genome, and the first channel for a respective patch can comprise a plurality of instances of a first plurality of parameters. Each instance of the first plurality of parameters can include a parameter for a methylation status of a respective CpG site in the corresponding independent set of CpG sites for the respective patch.

In this aspect, the at least one program can further comprise assigning all or a portion of each respective fragment in the plurality of fragments to a respective patch in the plurality of patches based upon a match between CpG sites of the respective fragment and the corresponding independent set of CpG sites of the single respective patch. In this aspect, the at least one program further comprises applying each respective patch in the plurality of patches to a corresponding trained model in a plurality of models thereby determining the cancer condition in the test subject.

Another aspect of the present disclosure provides a non-transitory computer-readable storage medium storing program code instructions that, when executed by a processor, cause the processor to perform a method of determining a cancer condition of a test subject of a species. The method can comprise obtaining a dataset, in electronic form, where the dataset comprises a corresponding methylation pattern of each respective fragment in a plurality of fragments. The corresponding methylation pattern of each respective fragment can be determined by a methylation sequencing of one or more nucleic acid samples of the respective fragment in a biological sample obtained from the test subject and comprises a methylation state of each CpG site in a corresponding plurality of CpG sites in the respective fragment.

In this aspect, the method further comprises obtaining a plurality of patches, where each respective patch in the plurality of patches comprises a first channel and represents a corresponding independent set of CpG sites in a reference genome of the species. Each respective CpG site in the corresponding independent set of CpG sites can correspond to a predetermined location in the reference genome. The first channel for a respective patch can comprise a plurality of instances of a first plurality of parameters, and each instance of the first plurality of parameters can include a parameter for a methylation status of a respective CpG site in the corresponding independent set of CpG sites for the respective patch.

In this aspect, the method further comprises assigning all or a portion of each respective fragment in the plurality of fragments to a respective patch in the plurality of patches based upon a match between CpG sites of the respective fragment and the corresponding independent set of CpG sites of the single respective patch. In this aspect, the method further comprises applying each respective patch in the plurality of patches to a corresponding trained model in a plurality of models thereby determining the cancer condition in the test subject.

In another aspect, a method of determining a cancer condition of a test subject of a species comprises obtaining, via one or more processors, a training dataset from one or more training subjects, wherein the training dataset comprises one or more training methylation patterns of a plurality of fragments in one or more biological samples obtained from the one or more training subjects and one or more predetermined cancer conditions associated with the one or more training methylation patterns; constructing, via the one or more processors, one or more patches based on the training dataset, each patch of the one or more patches comprising one or more channels and representing one or more CpG sites in a reference genome of the species, each CpG site of the one or more CpG sites corresponding to a predetermined location in the reference genome; training, via the one or more processors, a computational model based on the one or more patches and the training dataset; obtaining, via the one or more processors, a test dataset from the test subject, wherein the test dataset comprises one or more testing methylation patterns of a plurality of fragments in one or more biological samples obtained from the test subject; and determining, via the one or more processors, the cancer condition of the test subject based on the test dataset and the computational model.

Other embodiments are directed to systems, portable consumer devices, and computer readable media associated with methods described herein. As disclosed herein, any embodiment disclosed herein when applicable can be applied to any aspect.

Additional aspects and advantages of the present disclosure will become readily apparent to those skilled in this art from the following detailed description, where only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications herein are incorporated by reference in their entireties. In the event of a conflict between a term herein and a term in an incorporated reference, the term herein controls.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the several views of the drawings.

FIG. 1 is an exemplary flowchart describing a process of sequencing a fragment of cell-free (cf) DNA to obtain a methylation state vector, according to one or more embodiments of the present disclosure.

FIG. 2 is an illustration of the process of FIG. 1 of sequencing a fragment of cfDNA to obtain a methylation state vector, according to one or more embodiments of the present disclosure.

FIG. 3 illustrates an exemplary method of removing a respective fragment from a plurality of fragments based on a p-value, according to one or more embodiments of the present disclosure.

FIG. 4 illustrates an exemplary methylation pattern pipeline that includes a classifier, according to one or more embodiments of the present disclosure.

FIG. 5A illustrates an exemplary system for determining a disease condition of a test subject of a species, according to one or more embodiments of the present disclosure.

FIG. 5B illustrates an exemplary processing system for determining a disease condition of a test subject of a species, according to one or more embodiments of the present disclosure.

FIGS. 6A, 6B, 6C, 6D, 6E, 6F, 6G, 6H, 6I, 6J, 6K, 6L, 6M, and 6N illustrate exemplary patches, according to one or more embodiments of the present disclosure.

FIGS. 7A and 7B illustrate an exemplary patch classifier, according to one or more embodiments of the present disclosure.

FIGS. 8A and 8B provide exemplary methods for determining a cancer condition of a test subject of a species according to one or more embodiments of the present disclosure.

FIG. 9A illustrates exemplary genomic regions used in a patch CNN classifier, according to one or more embodiments of the present disclosure.

FIG. 9B illustrates exemplary cancer types used in a patch CNN classifier, according to one or more embodiments of the present disclosure.

FIG. 9C illustrates an example of the performance of a patch CNN classifier, according to one or more embodiments of the present disclosure.

FIG. 10A illustrates an example of the performance of a patch CNN classifier using a dataset in which 53 percent sensitivity (accuracy) at 99 percent specificity for detecting cancer (across all cancer types and stages) was achieved, according to one or more embodiments of the present disclosure.

FIG. 10B illustrates an example of the sensitivity of a patch CNN classifier in the binary setting across all cancer types, in which the classifier exhibits 88.00 percent sensitivity at 98 percent specificity, 74.36% sensitivity at 99 percent specificity, and 44.23% sensitivity at 99.5 percent specificity on CCGA 1 training of cfDNA samples, according to one or more embodiments of the present disclosure.

FIG. 11 illustrates an example of taking embedding values (activations) from each patch and clustering them using Isomap clustering, showing that the different cancer labels cluster to different regions of the Isomap, indicating that the embedding values discriminate cancer type according to one or more embodiments of the present disclosure.

FIG. 12 illustrates an example of the frequency of activation of the embedding layers of the 544 patches of a classifier across a set of samples according to one or more embodiments of the present disclosure.

FIG. 13 illustrates an example of a t-SNE clustering of the embedding values (activations) of the top six activated patches of a classifier across a set of samples according to one or more embodiments of the present disclosure. The figure shows that the patch to the far right, by itself, is capable of discriminating several different cancer types.

FIG. 14 illustrates an example of a t-SNE clustering of the embedding values (activations) of the top three activated patches of a classifier across a set of samples according to one or more embodiments of the present disclosure.

FIG. 15 illustrates exemplary results of classification performance using patch-CNN architecture, according to one or more embodiments of the present disclosure.

FIG. 16 illustrates an example of the performance of a patch based classifier by high signal cancer type according to one or more embodiments of the present disclosure, in which each dot represents a subject from CCGA 2 and the classifier provides a probability that the subject has the type of cancer specified on the y-axis.

FIG. 17A illustrates an exemplary confusion matrix analysis for tissue of origin for a classifier according to one or more embodiments of the present disclosure showing over 80 percent of TOO accuracy across all four stages in a cohort of subjects that includes subjects for each of the cancer types illustrated in the Figure. Samples of indeterminate status are included in the analysis.

FIG. 17B illustrates another exemplary confusion matrix analysis for tissue of origin for a classifier according to one or more embodiments of the present disclosure showing nearly 90 percent of TOO accuracy across all four stages in a cohort of subjects that includes subjects for each of the cancer types illustrated in the Figure. Samples of indeterminate status are excluded from the analysis.

FIG. 18 illustrates an exemplary computation of a p-value for a methylation pattern according to one or more embodiments of the present disclosure.

FIG. 19 illustrates an exemplary computer system 1901 that is programmed or otherwise configured to determine a disease condition of a test subject, according to one or more embodiments of the present disclosure.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

### I. Overview

Targeted methylation assays can provide a basis for computationally tractable systems and methods for classification of biological samples. For example, a limited subset of DNA sequencing base reads (e.g., approximately 3 billion in human cells) can be obtained using methylation sequencing (e.g., approximately 28 million CpG sites). Such CpG sites can serve as binary “switches” that toggle certain functions or direct cells in biological samples to specialize (e.g., a brain cell, a lung cell, a kidney cell, and/or a skin cell, among others). The regulation of methylation groups can be further characterized as a molecular marker for the detection of cancers. Moreover, because CpG sites play a role in cell specialization, their methylation pattern can be used to predict the origin (e.g., tissue of origin) of specific cell samples and/or DNA fragments. The use of CpG sites therefore can provide a distinct advantage over DNA base reads for the classification and characterization of biological samples.

Systems and methods can be provided for the detection and classification of a cancer condition of a test subject using methylation sequencing of nucleic acid samples and patch convolutional neural networks. A dataset can be obtained that comprises the methylation patterns of fragments determined by methylation sequencing, where a methylation pattern includes a methylation state of each CpG site in a plurality of CpG sites in a respective fragment. A first patch can be constructed based on the dataset. The first patch can represent a first independent set of CpG sites in a reference genome of the test subject species and comprise a first channel including a plurality of instances of a first plurality of parameters for a methylation status of respective CpG sites. The first patch can be constructed by populating, for each respective fragment that aligns to the first independent set of CpG sites, an instance of all or a portion of the first plurality of parameters based on the methylation pattern of the fragment. The cancer condition in the test subject can be determined by applying at least the first patch to a classifier. CfDNA fragments from a test subject can be treated to convert unmethylated cytosines to uracils, sequenced and the sequence reads can be compared to a reference genome to identify the methylation states at one or more CpG sites within the fragments. Identification of anomalously methylated cfDNA fragments, in comparison to healthy subjects, can provide insight into a subject's cancer status. DNA methylation anomalies (compared to healthy controls) can cause different effects, which may contribute to cancer. Various challenges can arise in the identification of anomalously methylated cfDNA fragments. First, determining one or more cfDNA fragments to be anomalously methylated can hold weight in comparison with a group of control subjects with fragments assumed to be normally methylated. Additionally, among a group of control subjects, methylation state can vary and this can be difficult to account for when evaluating whether a subject's cfDNA is anomalously methylated. Also, methylation of a cytosine at a CpG site causally can influence methylation at a subsequent CpG site.

Methylation can occur in deoxyribonucleic acid (DNA) when a hydrogen atom on the pyrimidine ring of a cytosine base is converted to a methyl group, forming 5-methylcytosine. In particular, methylation may occur at dinucleotides of cytosine and guanine referred to herein as “CpG sites.” Methylation may occur, although rare, at a cytosine not part of a CpG site or at another nucleotide that is not cytosine. Anomalous cfDNA fragment methylation may further be identified as hypermethylation or hypomethylation, both of which may be indicative of cancer status.

The principles described herein can be equally applicable for the detection of methylation in a non-CpG context, including non-cytosine methylation. The wet laboratory assays used to detect methylation may vary from those described herein. Further, the methylation state vectors may contain elements that are generally vectors of sites where methylation has or has not occurred (even if those sites are not CpG sites specifically). With that substitution, the remainder of the processes described herein can be the same, and consequently the inventive concepts described herein can be applicable to those other forms of methylation.

### II. Definitions

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. The term “about” or “approximately” can mean within an order of magnitude, within 5-fold, or within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

As used herein, the term “assay” refers to a technique for determining a property of a substance, e.g., a nucleic acid, a protein, a cell, a tissue, or an organ. An assay (e.g., a first assay or a second assay) can comprise a technique for determining the copy number variation of nucleic acids in a sample, the methylation status of nucleic acids in a sample, the fragment size distribution of nucleic acids in a sample, the mutational status of nucleic acids in a sample, or the fragmentation pattern of nucleic acids in a sample. Any assay can be used to detect any of the properties of nucleic acids mentioned herein. Properties of a nucleic acid can include a sequence, genomic identity, copy number, methylation state at one or more nucleotide positions, size of the nucleic acid, presence or absence of a mutation in the nucleic acid at one or more nucleotide positions, and pattern of fragmentation of a nucleic acid (e.g., the nucleotide position(s) at which a nucleic acid fragments). An assay or method can have a particular sensitivity and/or specificity, and their relative usefulness as a diagnostic tool can be measured using ROC-AUC statistics.

As used herein, the terms “biological sample,” “patient sample,” and “sample” are interchangeably used and refer to any sample taken from a subject, which can reflect a biological state associated with the subject. In some embodiments such samples contain cell-free nucleic acids such as cell-free DNA. In some embodiments, such samples include nucleic acids other than or in addition to cell-free nucleic acids. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. In some embodiments, the biological sample consists of blood, whole blood, plasma, serum, urine, cerebrospinal fluid, fecal, saliva, sweat, tears, pleural fluid, pericardial fluid, or peritoneal fluid of the subject. In such embodiments, the biological sample is limited to blood, whole blood, plasma, serum, urine, cerebrospinal fluid, fecal, saliva, sweat, tears, pleural fluid, pericardial fluid, or peritoneal fluid of the subject and does not contain other components (e.g., solid tissues, etc.) of the subject. A biological sample can include any tissue or material derived from a living or dead subject. A biological sample can be a cell-free sample. A biological sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof. The nucleic acid in the sample can be a cell-free nucleic acid. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological sample can be a stool sample. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis. A biological sample can be obtained from a subject invasively (e.g., surgical means) or non-invasively (e.g., a blood draw, a swab, or collection of a discharged sample).

As used herein, the term “cancer” or “tumor” refers to an abnormal mass of tissue in which the growth of the mass surpasses and is not coordinated with the growth of normal tissue. A cancer or tumor can be defined as “benign” or “malignant” depending on the following characteristics: degree of cellular differentiation including morphology and functionality, rate of growth, local invasion and metastasis. A “benign” tumor can be well differentiated, have characteristically slower growth than a malignant tumor and remain localized to the site of origin. In addition, a benign tumor does not have the capacity to infiltrate, invade or metastasize to distant sites. A “malignant” tumor can be a poorly differentiated (anaplasia), have characteristically rapid growth accompanied by progressive infiltration, invasion, and destruction of the surrounding tissue. Furthermore, a malignant tumor can have the capacity to metastasize to distant sites.

As used herein, the Circulating Cell-free Genome Atlas or “CCGA” is defined as an observational clinical study that prospectively collects blood and tissue from newly diagnosed cancer patients as well as blood from subjects who do not have a cancer diagnosis. The purpose of the study is to develop a pan-cancer classifier that distinguishes cancer from non-cancer and identifies tissue of origin. Example 1 provides further details of the CCGA 1 and CCGA 2 datasets.

As used herein the term “classification” can refer to any number(s) or other characters(s) that are associated with a particular property of a sample. For example, a “+” symbol (or the word “positive”) can signify that a sample is classified as having deletions or amplifications. In another example, the term “classification” can refer to an amount of tumor tissue in the subject and/or sample, a size of the tumor in the subject and/or sample, a stage of the tumor in the subject, a tumor load in the subject and/or sample, and presence of tumor metastasis in the subject. The classification can be binary (e.g., positive or negative) or have more levels of classification (e.g., a scale from 1 to 10 or 0 to 1). The terms “cutoff” and “threshold” can refer to predetermined numbers used in an operation. For example, a cutoff size can refer to a size above which fragments are excluded. A threshold value can be a value above or below which a particular classification applies. Either of these terms can be used in either of these contexts.

As used herein, the terms “nucleic acid” and “nucleic acid molecule” are used interchangeably. The terms refer to nucleic acids of any composition form, such as deoxyribonucleic acid (DNA, e.g., complementary DNA (cDNA), genomic DNA (gDNA) and the like), and/or DNA analogs (e.g., containing base analogs, sugar analogs and/or a non-native backbone and the like), all of which can be in single- or double-stranded form. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single-stranded, double-stranded and the like). A nucleic acid in some embodiments can be from a single chromosome or fragment thereof (e.g., a nucleic acid sample may be from one chromosome of a sample obtained from a diploid organism). In certain embodiments nucleic acids comprise nucleosomes, fragments or parts of nucleosomes or nucleosome-like structures. Nucleic acids sometimes comprise protein (e.g., histones, DNA binding proteins, and the like). Nucleic acids analyzed by processes described herein sometimes are substantially isolated and are not substantially associated with protein or other molecules. Nucleic acids also include derivatives, variants and analogs of DNA synthesized, replicated or amplified from single-stranded (“sense” or “antisense,” “plus” strand or “minus” strand, “forward” reading frame or “reverse” reading frame) and double-stranded polynucleotides. Deoxyribonucleotides include deoxyadenosine, deoxycytidine, deoxyguanosine and deoxythymidine. A nucleic acid may be prepared using a nucleic acid obtained from a subject as a template.

As used herein, the term “cell-free nucleic acids” refers to nucleic acid molecules that can be found outside cells, in bodily fluids such as blood, whole blood, plasma, serum, urine, cerebrospinal fluid, fecal, saliva, sweat, sweat, tears, pleural fluid, pericardial fluid, or peritoneal fluid of a subject. Cell-free nucleic acids originate from one or more healthy cells and/or from one or more cancer cells Cell-free nucleic acids are used interchangeably as circulating nucleic acids. Examples of the cell-free nucleic acids include but are not limited to RNA, mitochondrial DNA, or genomic DNA. As used herein, the terms “cell free nucleic acid,” “cell free DNA,” and “cfDNA” are used interchangeably. As used herein, the term “circulating tumor DNA” or “ctDNA” refers to nucleic acid fragments that originate from tumor cells or other types of cancer cells, which may be released into a fluid from an individual's body (e.g., bloodstream) as result of biological processes such as apoptosis or necrosis of dying cells or actively released by viable tumor cells.

As used herein, the term “fragment” is used interchangeably with the term “nucleic acid fragment” (e.g., a DNA fragment), and refers to a portion of a polynucleotide or polypeptide sequence that comprises at least three consecutive nucleotides. In the context of sequencing of nucleic cell-free nucleic acid fragments found in a biological sample, the terms “fragment” and “nucleic acid fragment” interchangeably refer to a cell-free nucleic acid molecule that is found in the biological sample or a representation thereof. In such a context, sequencing data (e.g., sequence reads from whole genome sequencing, targeted sequencing, etc.) are used to derive one or more copies of all or a portion of such a nucleic acid fragment. Such sequence reads, which in fact may be obtained from sequencing of PCR duplicates of the original nucleic acid fragment, therefore “represent” or “support” the nucleic acid fragment. There may be a plurality of sequence reads that each represent or support a particular nucleic acid fragment in the biological sample (e.g., PCR duplicates). Nucleic acid fragments can be considered cell-free nucleic acids. In some embodiments, one copy of a nucleic acid fragment is used to represent the original cell-free nucleic acid molecule (e.g., duplicates are removed through molecular identifiers that are attached to the cell-free nucleic acid molecule during the library preparation process). In some embodiments, methylation sequencing data can be used to further distinguish these nucleic acid fragments. For example, two nucleic acid fragments that share identical or near identical sequences may still correspond to different original cell-free nucleic acid molecules if they each harbor a different methylation pattern.

As used herein, the phrase “healthy” refers to a subject possessing good health. A healthy subject can demonstrate an absence of any malignant or non-malignant disease. A “healthy individual” can have other diseases or conditions, unrelated to the condition being assayed, which can normally not be considered “healthy.”

As used herein, the term “level of cancer” refers to whether cancer exists (e.g., presence or absence), a stage of a cancer, a size of tumor, presence or absence of metastasis, an estimated tumor fraction concentration, a total tumor mutational burden value, the total tumor burden of the body, and/or other measure of a severity of a cancer (e.g., recurrence of cancer). The level of cancer can be a number or other indicia, such as symbols, alphabet letters, and colors. The level can be zero. The level of cancer can also include premalignant or precancerous conditions (states) associated with mutations or a number of mutations. The level of cancer can be used in various ways. For example, screening can check if cancer is present in someone who is not known previously to have cancer. Assessment can investigate someone who has been diagnosed with cancer to monitor the progress of cancer over time, study the effectiveness of therapies or to determine the prognosis. The prognosis can be expressed as the chance of a subject dying of cancer, or the chance of the cancer progressing after a specific duration or time, or the chance of cancer metastasizing. Detection can comprise ‘screening’ or can comprise checking if someone, with suggestive features of cancer (e.g., symptoms or other positive tests), has cancer. A “level of pathology” can refer to level of pathology associated with a pathogen, where the level can be as described above for cancer. When the cancer is associated with a pathogen, a level of cancer can be a type of a level of pathology.

As used herein a “methylome” can be a measure of an amount or extent of DNA modification involving a methyl group (e.g., methylation or hydroxymethylation modifications) at a plurality of sites or loci in a genome. The methylome can correspond to all or a part of a genome, a substantial part of a genome, or relatively small portion(s) of a genome. A methylation profile of a substantial part of the genome can be considered equivalent to the methylome. A methylome of interest can be a methylome of an organ that can contribute nucleic acid, e.g., DNA into a bodily fluid (e.g., a methylome of brain cells, a bone, lungs, heart, muscles, kidneys, etc.). The organ can be a transplanted organ.

As disclosed herein, the term “methylation” covers any type of modification involving a methyl group, including but not limited to hydroxymethylation. The “methylation density” of a region can be the number of reads at sites within a region showing methylation divided by the total number of reads covering the sites in the region. The sites can have specific characteristics, (e.g., the sites can be CpG sites). The “CpG methylation density” of a region can be the number of reads showing CpG methylation divided by the total number of reads covering CpG sites in the region (e.g., a particular CpG site, CpG sites within a CpG island, or a larger region). For example, the methylation density for each 100-kb bin in the human genome can be determined from the total number of unconverted cytosines (which can correspond to methylated cytosine) at CpG sites as a proportion of all CpG sites covered by sequence reads mapped to the 100-kb region. This analysis can also be performed for other bin sizes, e.g., 50-kb or 1-Mb, etc. A region can be an entire genome or a chromosome or part of a chromosome (e.g., a chromosomal arm).

“DNA methylation” in mammalian genomes can refer to the addition of a methyl group to position 5 of the heterocyclic ring of cytosine (e.g., to produce 5-methylcytosine) among CpG dinucleotides. Methylation of cytosine can occur in cytosines in other sequence contexts, for example 5′-CHG-3′ and 5′-CHH-3′, where H is adenine, cytosine or thymine. Cytosine methylation can also be in the form of 5-hydroxymethylcytosine. Methylation of DNA can include methylation of non-cytosine nucleotides, such as N6-methyladenine. For example, methylation data (e.g., density, distribution, pattern or level of methylation) from different genomic regions can be converted to one or more vector set and analyzed by methods and systems disclosed herein.

As used herein, the term “mutation,” refers to a detectable change in the genetic material of one or more cells. In a particular example, one or more mutations can be found in, and can identify, cancer cells (e.g., driver and passenger mutations). A mutation can be transmitted from apparent cell to a daughter cell. A person having skill in the art will appreciate that a genetic mutation (e.g., a driver mutation) in a parent cell can induce additional, different mutations (e.g., passenger mutations) in a daughter cell. A mutation generally occurs in a nucleic acid. In a particular example, a mutation can be a detectable change in one or more deoxyribonucleic acids or fragments thereof. A mutation generally refers to nucleotides that is added, deleted, substituted for, inverted, or transposed to a new position in a nucleic acid. A mutation can be a spontaneous mutation or an experimentally induced mutation. A mutation in the sequence of a particular tissue is an example of a “tissue-specific allele.” For example, a tumor can have a mutation that results in an allele at a locus that does not occur in normal cells. Another example of a “tissue-specific allele” is a fetal-specific allele that occurs in the fetal tissue, but not the maternal tissue.

As used herein, the term “reference genome” refers to any particular known, sequenced or characterized genome, whether partial or complete, of any organism or virus that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38).

As used herein, the terms “sequencing,” “sequence determination,” and the like refers generally to any and all biochemical processes that may be used to determine the order of biological macromolecules such as nucleic acids or proteins. For example, sequencing data can include all or a portion of the nucleotide bases in a nucleic acid molecule such as a DNA fragment.

As used herein, the term “sequence reads” or “reads” refers to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). In some embodiments, sequence reads (e.g., single-end or paired-end reads) can be generated from one or both strands of a targeted nucleic acid fragment. The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore sequencing, for example, can provide sequence reads that can vary in size from tens to hundreds to thousands of base pairs. Illumina parallel sequencing can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

The terms “sequencing depth,” “coverage” and “coverage rate” are used interchangeably herein to refer to the number of times a locus is covered by a consensus sequence read corresponding to a unique nucleic acid target molecule (“nucleic acid fragment”) aligned to the locus; e.g., the sequencing depth is equal to the number of unique nucleic acid target fragments (excluding PCR sequencing duplicates) covering the locus. The locus can be as small as a nucleotide, or as large as a chromosome arm, or as large as an entire genome. Sequencing depth can be expressed as “YX”, e.g., 50×, 100×, etc., where “Y” refers to the number of times a locus is covered with a sequence corresponding to a nucleic acid target; e.g., the number of times independent sequence information is obtained covering the particular locus. In some embodiments, the sequencing depth corresponds to the number of genomes that have been sequenced. Sequencing depth can also be applied to multiple loci, or the whole genome, in which case Y can refer to the mean or average number of times a loci or a haploid genome, or a whole genome, respectively, is sequenced. When a mean depth is quoted, the actual depth for different loci included in the dataset can span over a range of values. Ultra-deep sequencing can refer to at least 100× in sequencing depth at a locus.

As used herein, the term “true positive” (TP) refers to a subject having a condition. “True positive” can refer to a subject that has a tumor, a cancer, a precancerous condition (e.g., a precancerous lesion), a localized or a metastasized cancer, or a non-malignant disease. “True positive” can refer to a subject having a condition, and is identified as having the condition by an assay or method of the present disclosure.

As used herein, the term “true negative” (TN) refers to a subject that does not have a condition or does not have a detectable condition. True negative can refer to a subject that does not have a disease or a detectable disease, such as a tumor, a cancer, a precancerous condition (e.g., a precancerous lesion), a localized or a metastasized cancer, a non-malignant disease, or a subject that is otherwise healthy. True negative can refer to a subject that does not have a condition or does not have a detectable condition, or is identified as not having the condition by an assay or method of the present disclosure.

As used herein, the term “sensitivity” or “true positive rate” (TPR) refers to the number of true positives divided by the sum of the number of true positives and false negatives. Sensitivity can characterize the ability of an assay or method to correctly identify a proportion of the population that truly has a condition. For example, sensitivity can characterize the ability of a method to correctly identify the number of subjects within a population having cancer. In another example, sensitivity can characterize the ability of a method to correctly identify the one or more markers indicative of cancer.

As used herein, the term “specificity” or “true negative rate” (TNR) refers to the number of true negatives divided by the sum of the number of true negatives and false positives. Specificity can characterize the ability of an assay or method to correctly identify a proportion of the population that truly does not have a condition. For example, specificity can characterize the ability of a method to correctly identify the number of subjects within a population not having cancer. In another example, specificity can characterize the ability of a method to correctly identify one or more markers indicative of cancer.

As used herein, the term “false positive” (FP) refers to a subject that does not have a condition. False positive can refer to a subject that does not have a tumor, a cancer, a precancerous condition (e.g., a precancerous lesion), a localized or a metastasized cancer, a non-malignant disease, or is otherwise healthy. The term false positive can refer to a subject that does not have a condition, but is identified as having the condition by an assay or method of the present disclosure. As used herein, the term “false negative” (FN) refers to a subject that has a condition. False negative can refer to a subject that has a tumor, a cancer, a precancerous condition (e.g., a precancerous lesion), a localized or a metastasized cancer, or a non-malignant disease. The term false negative can refer to a subject that has a condition, but is identified as not having the condition by an assay or method of the present disclosure.

As used herein, the term “single nucleotide variant” or “SNV” refers to a substitution of one nucleotide to a different nucleotide at a position (e.g., site) of a nucleotide sequence, e.g., a sequence read from an individual. A substitution from a first nucleobase X to a second nucleobase Y may be denoted as “X>Y.” For example, a cytosine to thymine SNV may be denoted as “C>T.”

As used herein, the terms “size profile” and “size distribution” can relate to the sizes of DNA fragments in a biological sample. A size profile can be a histogram that provides a distribution of an amount of DNA fragments at a variety of sizes. Various statistical parameters (also referred to as size parameters or just parameter) can distinguish one size profile to another. One parameter can be the percentage of DNA fragment of a particular size or range of sizes relative to all DNA fragments or relative to DNA fragments of another size or range.

As used herein, the term “subject” refers to any living or non-living organism, including but not limited to a human (e.g., a male human, female human, fetus, pregnant female, child, or the like), a non-human animal, a plant, a bacterium, a fungus or a protist. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any stage (e.g., a man, a women or a child).

As used herein, the term “tissue” can correspond to a group of cells that group together as a functional unit. More than one type of cell can be found in a single tissue. Different types of tissue may comprise different types of cells (e.g., hepatocytes, alveolar cells or blood cells), but also can correspond to tissue from different organisms (mother versus fetus) or to healthy cells versus tumor cells. The term “tissue” can refer to any group of cells found in the human body (e.g., heart tissue, lung tissue, kidney tissue, nasopharyngeal tissue, oropharyngeal tissue). The term “tissue” or “tissue type” can be used to refer to a tissue from which a cell-free nucleic acid originates. In one example, viral nucleic acid fragments can be derived from blood tissue. In another example, viral nucleic acid fragments can be derived from tumor tissue.

As used herein, the term “vector” is an enumerated list of elements, such as an array of elements, where each element has an assigned meaning. As such, the term “vector” as used in the present disclosure is interchangeable with the term “tensor.” As an example, if a vector comprises the bin counts for 10,000 bins, there exists a predetermined element in the vector for each one of the 10,000 bins. For ease of presentation, in some instances a vector may be described as being one-dimensional. However, the present disclosure is not so limited. A vector of any dimension may be used in the present disclosure provided that a description of what each element in the vector represents is defined (e.g., that element 1 represents bin count of bin 1 of a plurality of bins, etc.).

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

### III. Sample Processing

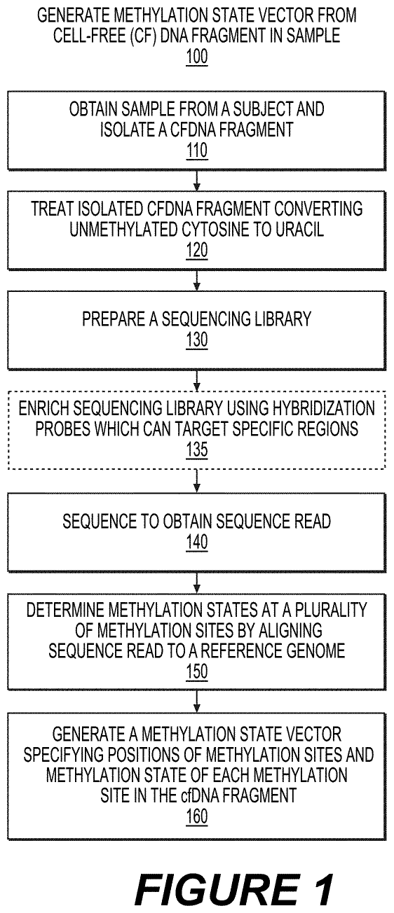

FIG. 1 is an exemplary flowchart describing a process 100 of sequencing a fragment of cell-free (cf) DNA to obtain a methylation state vector. An analytics system (or a processing system described elsewhere herein) can first obtain 110 a sample from a subject comprising a plurality of cfDNA fragments. Generally, samples may be from healthy subjects, subjects known to have or suspected of having cancer, or subjects where no prior information is known. The sample (e.g., either testing sample or training sample) can be selected from blood, plasma, serum, urine, fecal, and/or saliva samples. Alternatively, the sample can be selected from whole blood, a blood fraction, a tissue biopsy, pleural fluid, pericardial fluid, cerebrospinal fluid, or peritoneal fluid.

From the sample, the cfDNA fragments can be treated to convert unmethylated cytosines to uracils 120. The method can use a bisulfite treatment of the cfDNA fragments which converts the unmethylated cytosines to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, Calif.)) can be used for the bisulfite conversion. The conversion of unmethylated cytosines to uracils can be accomplished using an enzymatic reaction. For example, the conversion can use a commercially available kit for conversion of unmethylated cytosines to uracils, such as APOBEC-Seq (NEBiolabs, Ipswich, Mass.).

From the converted cfDNA fragments, a sequencing library can be prepared 130. Optionally, the sequencing library may be enriched 135 for cfDNA fragments, or genomic regions, that are informative for cancer status using a plurality of hybridization probes. The hybridization probes can be short oligonucleotides capable of hybridizing to targeted cfDNA fragments, or to cfDNA fragments derived from one or more targeted regions, and enriching for those fragments or regions for subsequent sequencing and analysis. Hybridization probes may be used to perform a targeted, high-depth analysis of a set of specified CpG sites of interest. Once prepared, the sequencing library or a portion thereof can be sequenced to obtain a plurality of sequence reads 140. The sequence reads may be in a computer-readable, digital format for processing and interpretation by computer software. A plurality of samples can be prepared and sequenced concurrently. The plurality of samples can include at least 10, 20, 50, 96, 100, 200, 500, 1000, 10000 or more samples.

From the sequence reads, the analytics system can determine 150 a location and methylation state for each of one or more CpG sites based on alignment to a reference genome. The analytics system can generate 160 a methylation state vector for each fragment specifying a location of the fragment in the reference genome (e.g., as specified by the position of the first CpG site in each fragment, or another similar metric), a number of CpG sites in the fragment, and the methylation state of each CpG site in the fragment whether methylated (e.g., denoted as M), unmethylated (e.g., denoted as U), or indeterminate (or other as described elsewhere herein, e.g., denoted as I). Observed states can include states of methylated and unmethylated; whereas, an unobserved state is indeterminate. The methylation state vectors may be stored in temporary or persistent computer memory for later use and processing. Further, the analytics system may remove duplicate reads or duplicate methylation state vectors from a single subject. The analytics system can perform contamination detection (e.g., human sources of contamination, unexpected germline haplotypes, cross-sample contamination, probe contamination, biological contamination, and/or technician contamination). The analytics system can assess quality control metrics (e.g., for enrichment, pull-down, coverage, and/or alignment). The analytics system may determine that a certain fragment has one or more CpG sites that have an indeterminate methylation state. Indeterminate methylation states may originate from sequencing errors and/or disagreements between methylation states of a DNA fragment's complementary strands. The analytics system may decide to exclude such fragments or selectively include such fragments but build a model accounting for such indeterminate methylation states. Excluding indeterminate samples from further tissue of origin analysis can enhance performance.

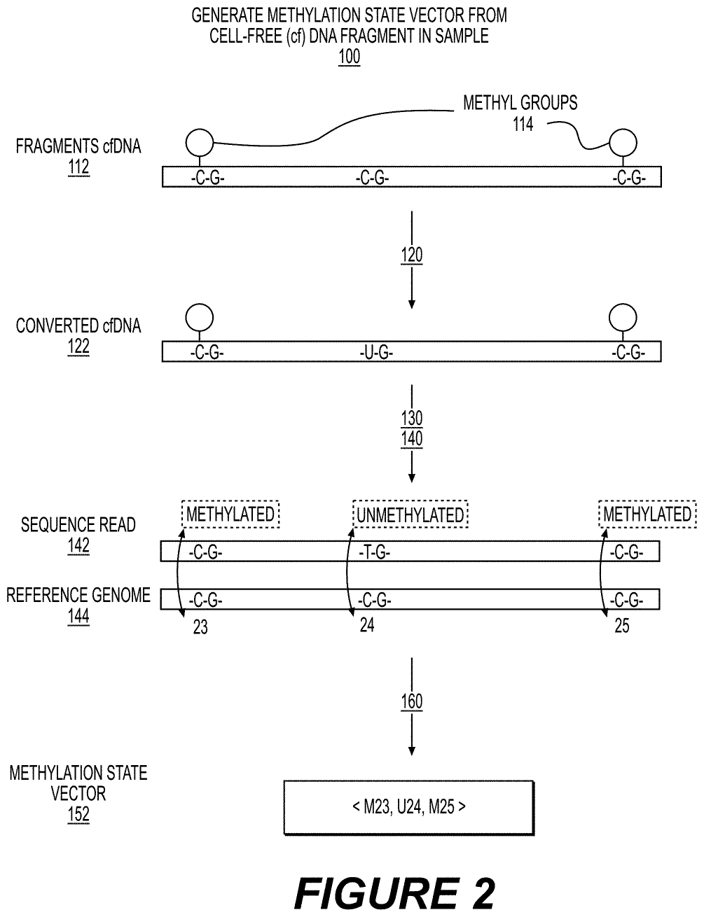

FIG. 2 is an illustration of the exemplary process 100 of FIG. 1 of sequencing a cfDNA fragment to obtain a methylation state vector. As an example, the analytics system can take a cfDNA fragment 112. The cfDNA fragment 112 can contain three CpG sites. As shown, the first and third CpG sites of the cfDNA fragment 112 can be methylated 114. During the treatment step 120, the cfDNA fragment 112 can be converted to generate a converted cfDNA fragment 122. During the treatment 120, the second CpG site, which is unmethylated, can have its cytosine converted to uracil, while the first and third CpG sites may not be converted.

After conversion, a sequencing library 130 can be prepared and sequenced 140 generating a sequence read 142. The analytics system can align 150 the sequence read 142 to a reference genome 144. The reference genome 144 can provide the context as to what position in a human genome the fragment cfDNA originates from. The analytics system can align 150 the sequence read such that the three CpG sites correlate to CpG sites 23, 24, and 25 (arbitrary reference identifiers used for convenience of description). The analytics system can thus generate information both on methylation state of all CpG sites on the cfDNA fragment 112 and to which position in the human genome the CpG sites map. As shown, the CpG sites on sequence read 142 which are methylated can be read as cytosines. The cytosines can appear in the sequence read 142 in the first and third CpG site which allows one to infer that the first and third CpG sites in the original cfDNA fragment are methylated. Whereas, the second CpG site can be read as a thymine (U is converted to T during the sequencing process), and thus, one can infer that the second CpG site is unmethylated in the original cfDNA fragment. With these two pieces of information, the methylation state and location, the analytics system can generate 160 a methylation state vector 152 for the cfDNA fragment 112. The resulting methylation state vector 152 can be <M23, U24, M25>, where “M” corresponds to a methylated CpG site, “U” corresponds to an unmethylated CpG site, and the subscript number can correspond to a position of each CpG site in the reference genome.

As discussed further in Example 8 below, the identified methylation state vectors can undergo p-value filtration and classification, and the classification output can be compiled into a results report.

### IV. Example System

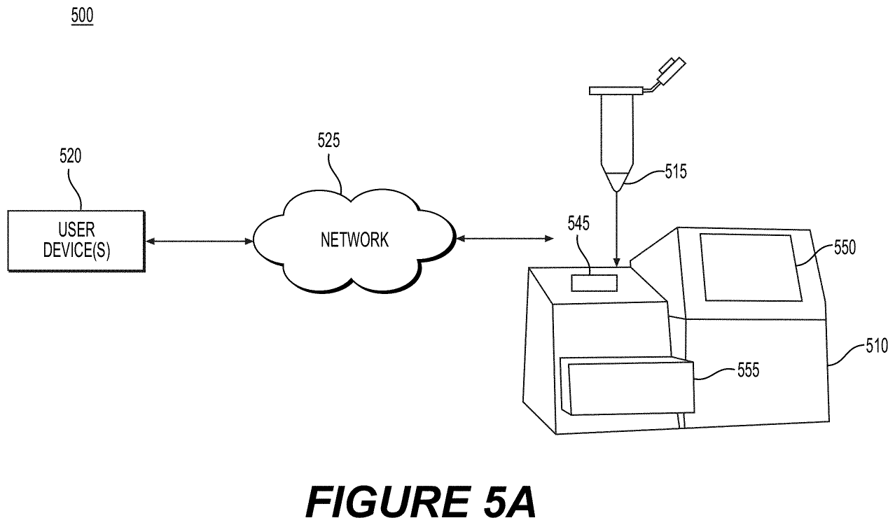

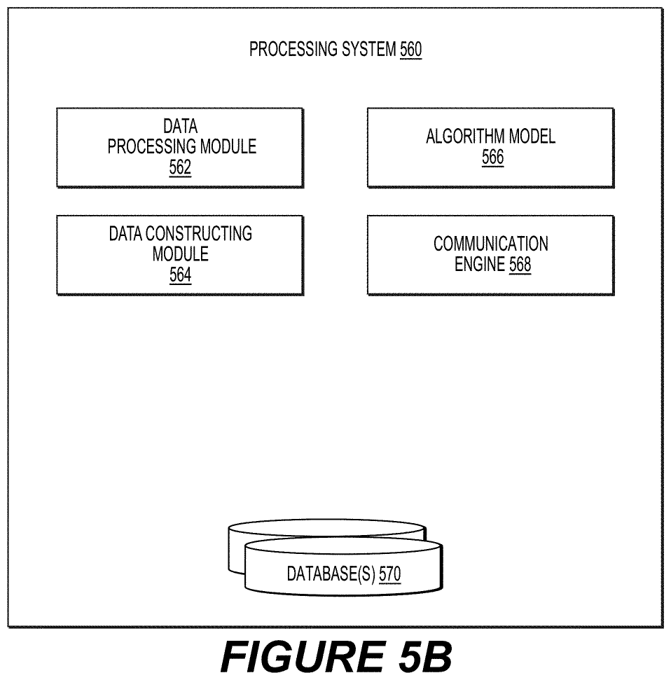

FIG. 5A depicts an exemplary environment/system in which a method of determining a disease/cancer condition of a test subject can be implemented. The environment 500 can include a sequencing device 510 and one or more user devices 520 connected via a network 525.

The sequencing device 510 can include a sample container 515, a flow cell 545, a graphical user interface 550, and one or more loading trays 555. The sample container 515 can be configured to carry, hold, and/or store one or more test and/or training samples. The flow cell 545 can be placed in a flow cell holder of the sequencing device 510. The flow cell 545 can be a solid support that can be configured to retain and/or allow the orderly passage of reagent solutions over bound analytes. The graphical user interface 550 can enable user interactions with particular tasks (e.g., loading samples and buffers in the loading trays, or obtaining sequencing data that comprises a dataset with corresponding methylation pattern). For instance, once a user (e.g., a test subject, a training subject, a health professional) has provided the reagents and enriched fragment samples to the loading trays 555 of the sequencing device 510, the user can initiate sequencing by interacting with the graphical user interface 550 of the sequencing device 510. The sequencing device 510 can include one or more processing systems describe elsewhere herein.

User devices 520 can each be a computer system, such as a laptop or desktop computer, or a mobile computing device such as a smartphone or tablet. The user devices 520 can be communicatively coupled with the sequencing device 510 via network 525. Each user device can process data obtained from the sequencing device 510 for various applications such as generating a report regarding a cancer condition to a user. The user can be a test subject, a training subject, or anyone can have access to the report (e.g., health professionals). The user devices 520 can include one or more processing systems describe elsewhere herein. The one or more user devices 520 can comprise a processing system and memory storing computer instructions that, when executed by the processing system, cause the processing system to perform one or more steps of any of the methods or processes disclosed herein.

The network 525 can be configured to provide communication between various components or devices shown in FIG. 5A. The network 525 can be implemented as the Internet, a wireless network, a wired network, a local area network (LAN), a Wide Area Network (WANs), Bluetooth, Near Field Communication (NFC), or any other type of network that provides communications between one or more components. The network 525 can be implemented using cell and/or pager networks, satellite, licensed radio, or a combination of licensed and unlicensed radio. The network 525 can be wireless, wired, or a combination thereof. The network 525 can be a public network (e.g., the internet), a private network (e.g., a network within an organization), or a combination of public and private networks.

FIG. 5B depicts an exemplary block diagram of a processing system 560 for determining a disease/cancer condition of a test subject. The processing system 560 can comprise one or more processors or servers that perform one or more steps of any of the methods or processes disclosed herein. The processing system 560 can include a plurality of models, engines, and modules. As shown in FIG. 5B, the processing system 560 can include a data processing module 562, a data constructing module 564, an algorithm model 566, a communication engine 568, and one or more databases 570.

The data processing module 562 can be configured to clean, process, manage, convert, and/or transform data obtained from the sequencing device 510. In one example, the data processing module can convert the data obtained from the sequencing device to data that can be used and/or recognized by other modules, engines, or models. For instance, the data constructing module 564 can construct output data from the data processing module 562. The data constructing module 564 can be configured to construct and/or further process data (e.g., construct one or more patches described elsewhere herein) obtained from the sequencing device 510 or any module, model, and engine of the processing system. In one example, the data constructing module 566 can prune a plurality of fragments by removing from the plurality of fragments each respective fragment.

The algorithm model 568 can be configured to analyze, translate, convert, model, and/or transform data via one or more algorithms or models. Such algorithms or models can include any computational, mathematical, statistical, or machine learning algorithms, such as a classifier or a computational model described elsewhere herein. The classifier or the computational model can include at least one convolutional neural network patch. The classifier or computational model can comprise a first stage model and a second stage model. The first stage model can sequentially receive a plurality of vector sets and provide a plurality of output scores, and the second stage model can receive a vector set provided by the first stage model and provides an output score. The classifier or the computational model can include a layer that receives input values and is associated with at least one filter comprising a set of filter weights. This layer can compute intermediate values as a function of: (i) the set of filter weights and (ii) the plurality of input values. The classifier or the computational model can be stored in the one or more databases (e.g., non-persistent memory or persistent memory).

The communication engine 568 can be configured to provide interfaces to one or more user devices (e.g., user devices 520), such as one or more keyboards, mouse devices, and the like, that enable the processing system 560 to receive data and/or any information from the one or more user devices 520 or sequencing device 510.

The one or more databases 570 can include one or more memory devices configured to store data (e.g., a pre-trained model, training datasets, etc.). Additionally, the one or more databases 570 can be implemented as a computer system with a storage device. The one or more databases 570 can be used by components of a system or a device (e.g., a sequencing device 510) to perform one or more operations. The one or more databases 570 can be co-located with the processing system 560, and/or co-located with one another on the network. Each of the one or more of databases 570 can be the same as or different from other databases. Each of the one or more of databases 564 can be located in the same location as or be remote from other databases. The one or more databases may store additional modules and data structures not described above or elsewhere herein.

The above identified components (e.g., modules) may not be implemented as separate software programs, procedures, datasets, or modules, and thus various subsets of these modules and data may be combined or otherwise re-arranged in various implementations. In some embodiments, one or more of the above identified elements can be stored in a computer system, other than that of system 500, that is addressable by system 500 so that system 500 may retrieve all or a portion of such data when needed.

### V. Example Methods

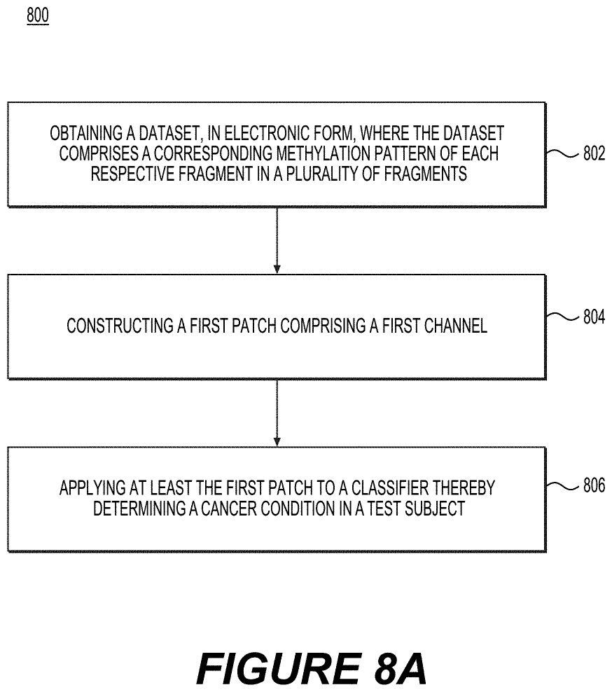

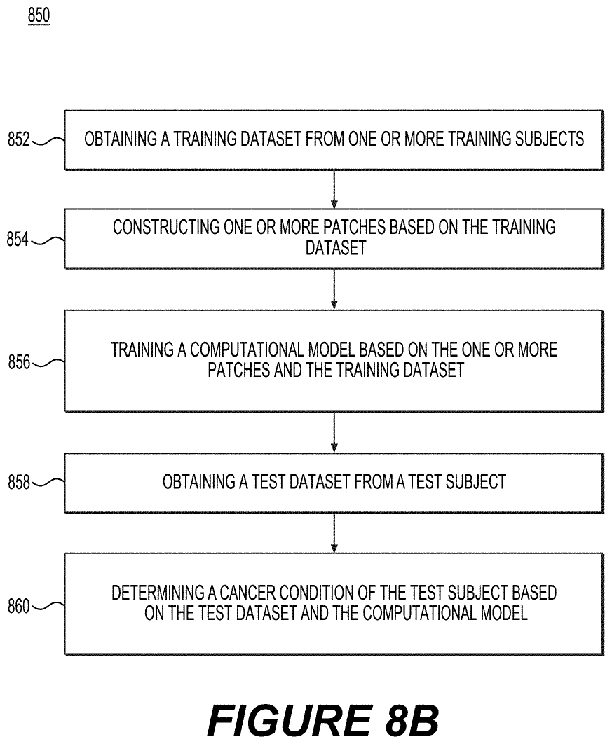

While a system in accordance with the present disclosure has been disclosed with reference to FIGS. 5A and 5B, an exemplary method 800 in accordance with the present disclosure is now detailed in conjunction with FIG. 8A. The method can be performed by the environment 500 and/or processing system 560 disclose herein.

Step 802 of the method 800 can include obtaining a dataset, in electronic form, where the dataset comprises a corresponding methylation pattern of each respective fragment in a plurality of fragments. The corresponding methylation pattern of each respective fragment can be determined by a methylation sequencing of one or more nucleic acid samples comprising the respective fragment in a biological sample obtained from the test subject. The corresponding methylation pattern of each respective fragment can comprise a methylation state of each CpG site in a corresponding plurality of CpG sites in the respective fragment.

Each fragment in the plurality of fragments can include a unique fragment whose nucleic acid sequence aligns (or maps) to a different genomic location or locations. Each fragment in the plurality of fragments can include a unique fragment that includes a different methylation pattern. The location that sequence reads for a fragment map to can be determined using a program such as BLAST, BLASR, BWA-MEM, DAMAPPER, NGMLR, GraphMap, Minimap, among others. BGREAT and deBGA can be both designed to work with second generation sequencing data. BlastGraph can use BLAST mapping results to cluster alignments and perform comparative genomic analyses. GramTools can map short reads to a population reference graph.

The methylation sequencing of one or more nucleic acid samples can include i) whole genome methylation sequencing, ii) whole genome bisulfite sequencing (WGBS), or iii) targeted DNA methylation sequencing using a plurality of nucleic acid probes. The methylation sequencing of one or more nucleic acid samples can include reduced representation bisulfite sequencing, methylated DNA immunoprecipitation sequencing, next-generation sequencing, pyrosequencing, methylation specific PCR, direct Sanger sequencing of bisulfite converted DNA, and/or Bisulfite Amplicon Sequencing (BSAS). The methylation sequencing can be performed using Nanopore sequencing or Illumina sequencing. The methylation sequencing of one or more nucleic acid samples can use a plurality of nucleic acid probes (e.g., less than 100 probes, between 100 and 1000 probes, between 500 and 10,000 probes, between 1000 and 50,000 probes, or more than 50,000 probes).

Targeted DNA methylation sequencing can be performed in various ways. Different enzymatic treatments and combinations with chemical treatment(s) can be used to convert either methylated cytosines or unmethylated cytosines. For example, the methylation sequencing of one or more nucleic acid samples can detect one or more 5-methylcytosine (5mC) and/or 5-hydroxymethylcytosine (5hmC) in the respective fragment. As another example, the methylation sequencing of one or more nucleic acid samples can comprise conversion of one or more unmethylated cytosines or one or more methylated cytosines, in the respective fragment, to a corresponding one or more uracils. The one or more uracils can be detected during the methylation sequencing as one or more corresponding thymines. The conversion of one or more unmethylated cytosines or one or more methylated cytosines can comprise a chemical conversion, an enzymatic conversion, or combinations of such.

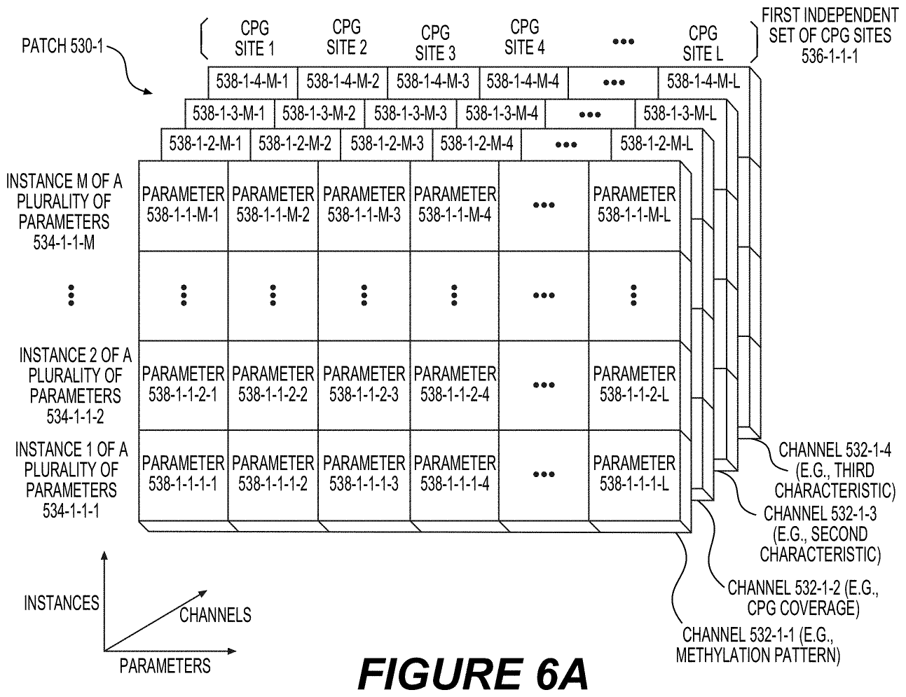

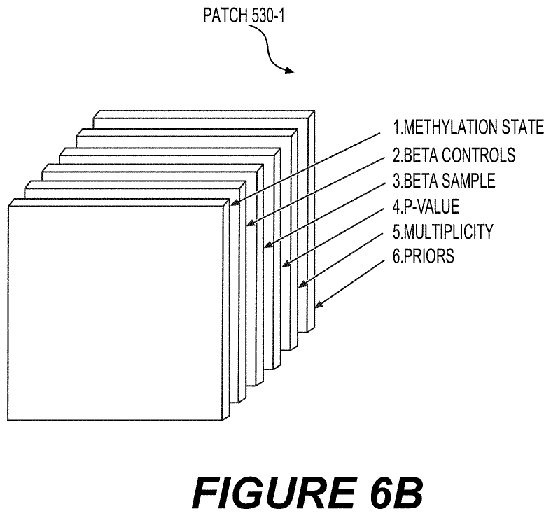

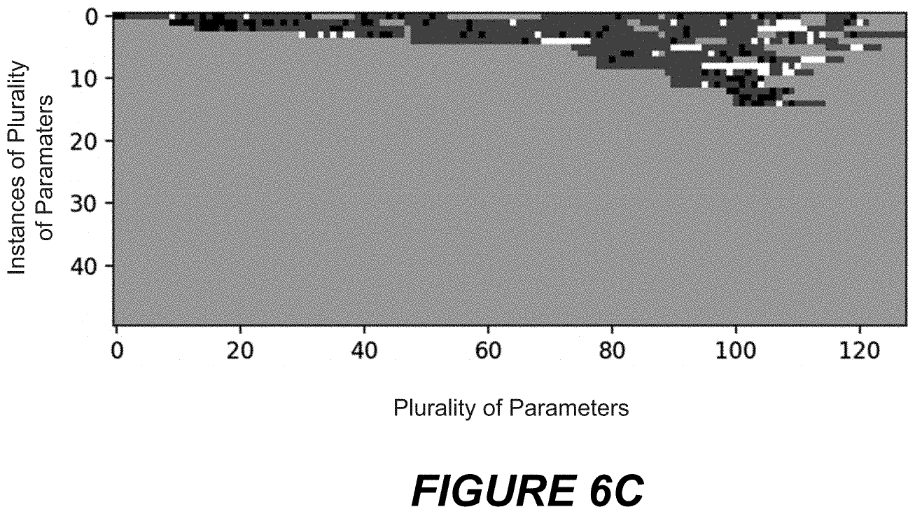

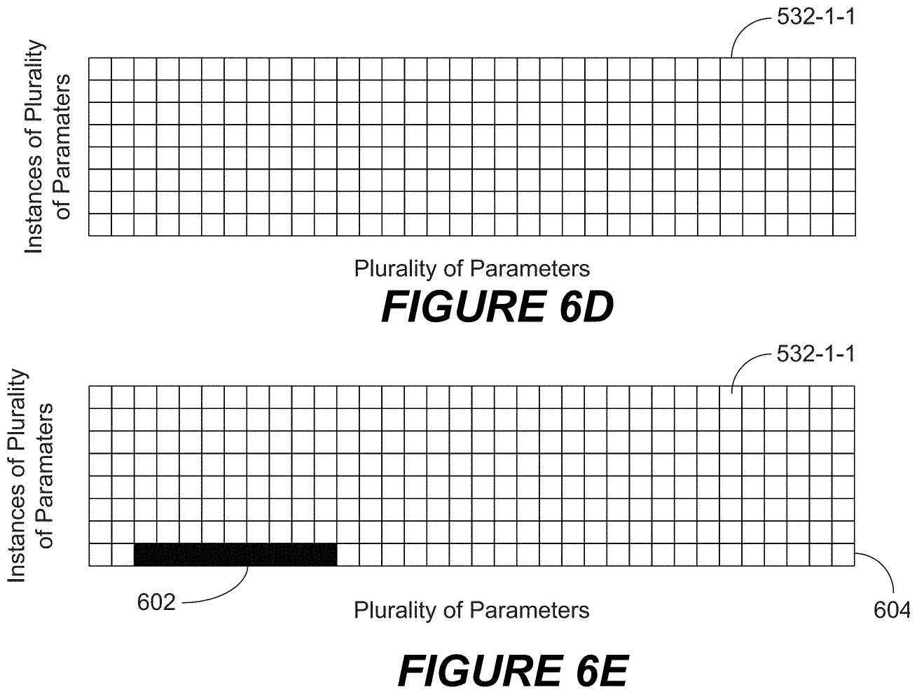

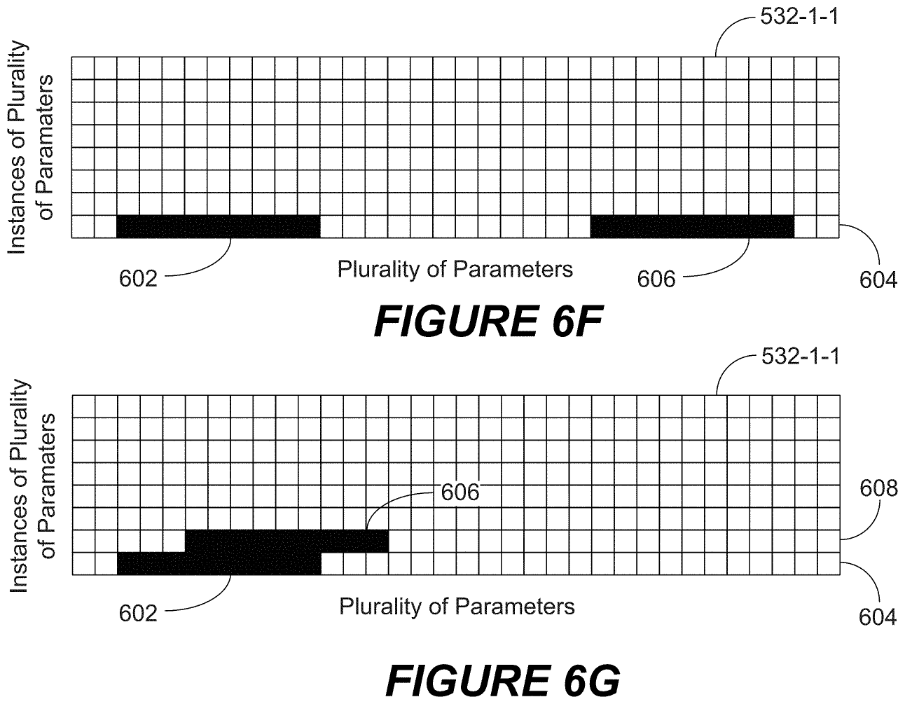

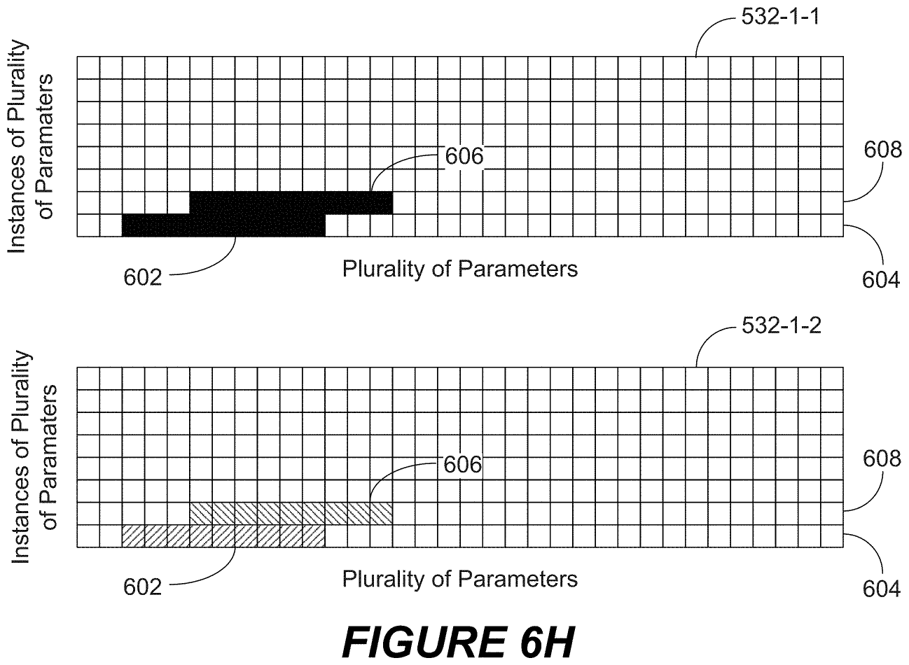

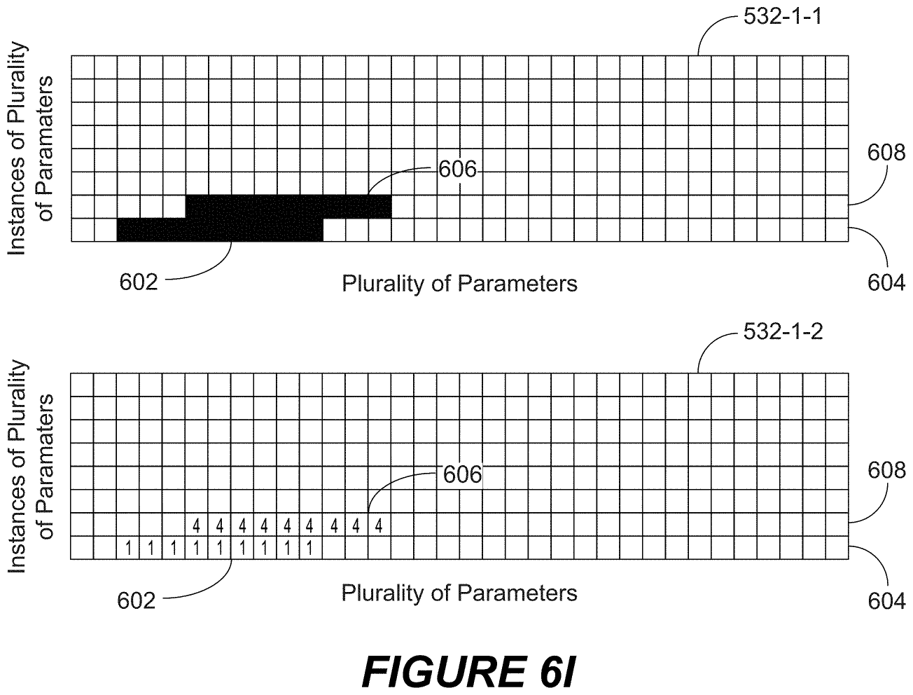

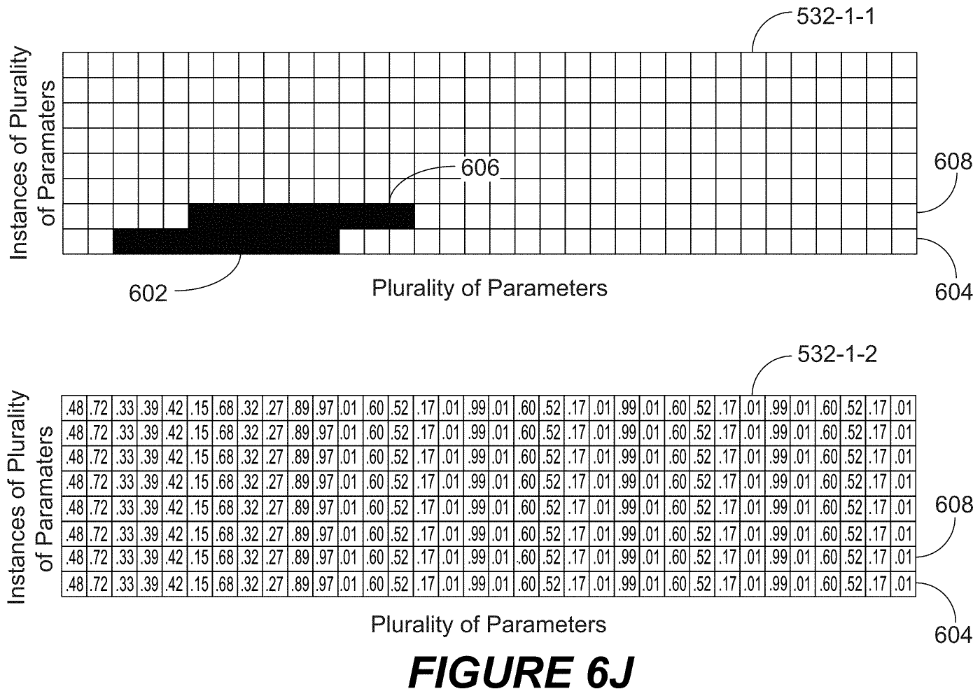

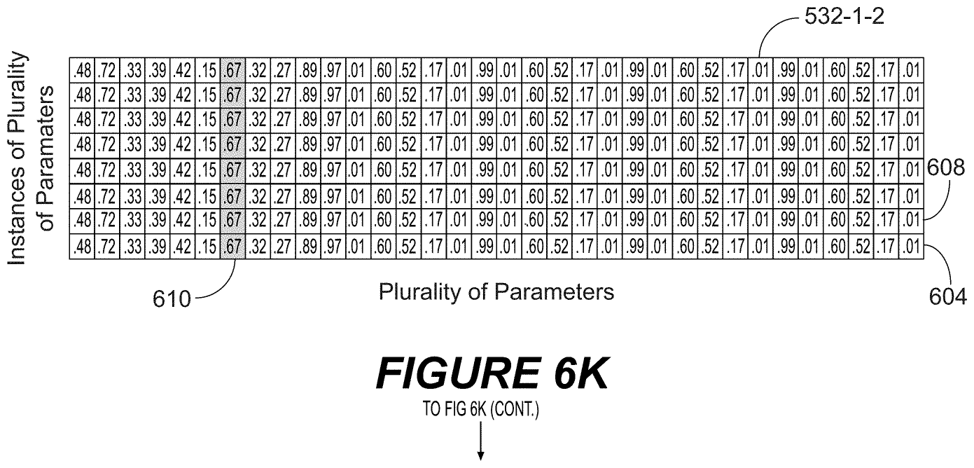

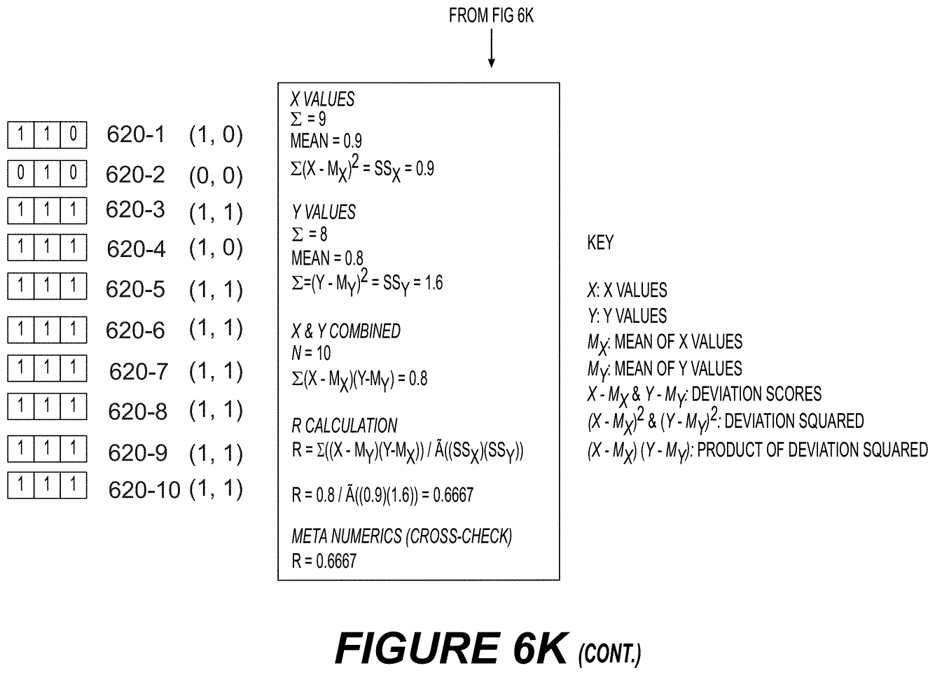

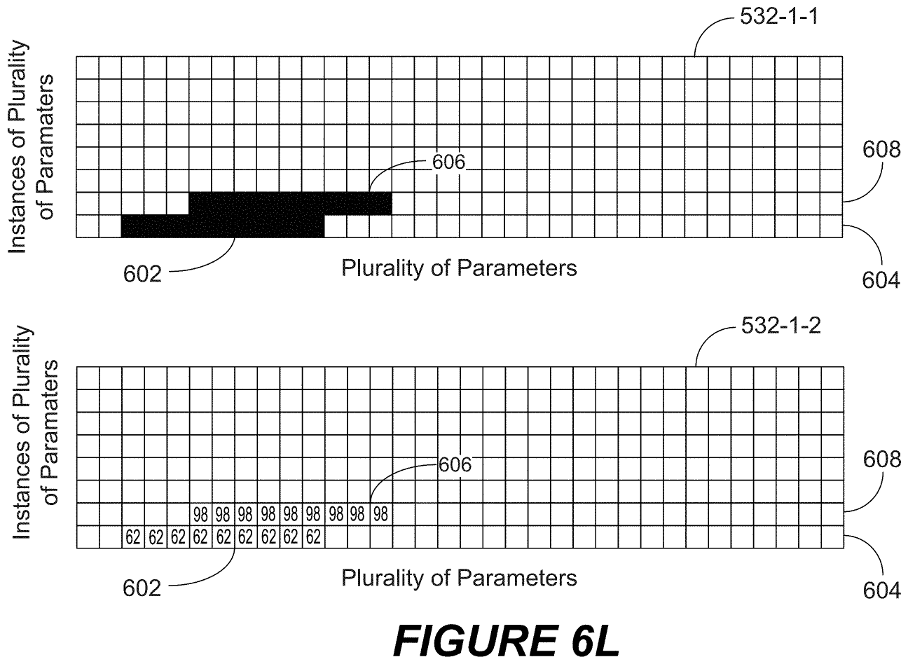

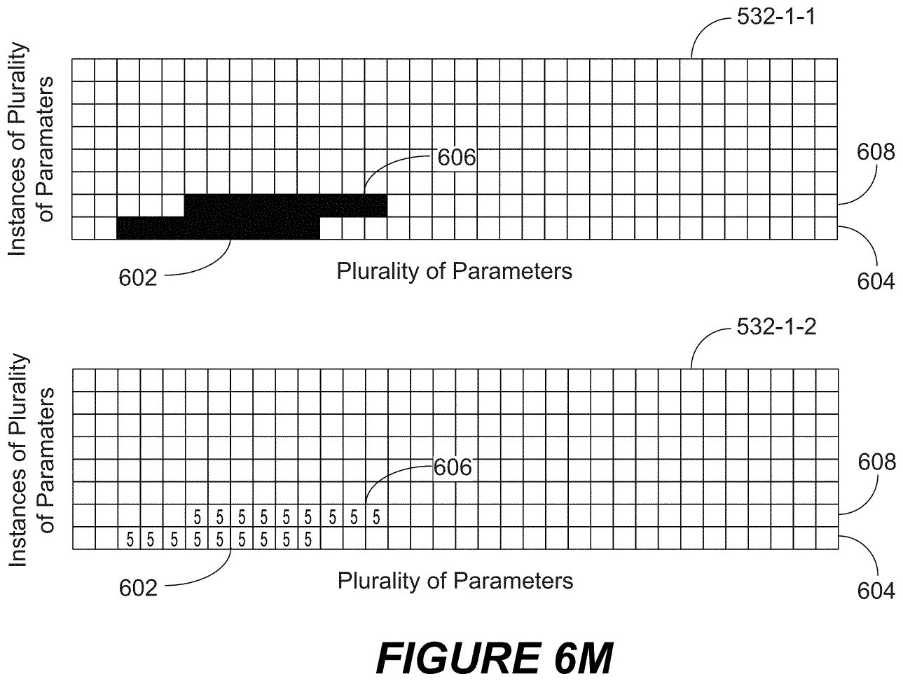

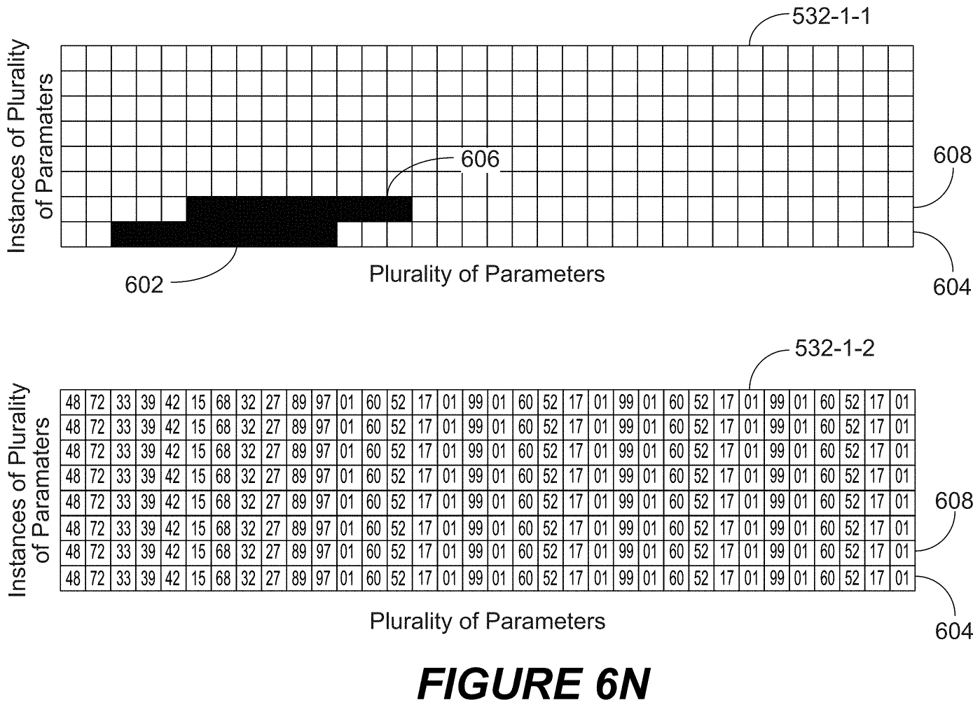

Step 804 of the method 800 can include constructing a first patch comprising a first channel. The first patch can represent a first independent set of CpG sites in a reference genome of the species. Each respective CpG site in the first independent set of CpG sites can correspond to a predetermined location in the reference genome. FIG. 6A illustrates the structure of an example first patch 530-1. The first patch 530-1 can comprise at least one channel (e.g., a first channel), where the first channel 532-1-1 can comprise a first independent set of CpG sites 536-1-1-1 including CpG sites 1 through L. Here, L can be a positive integer (e.g., 2, 3, 4, 5, 6, 7, 8, 9, 10 or more, 20 or more, 30 or more or 50 or more).

The first independent set of CpG sites can comprise a predetermined number of CpG sites. The first independent set of CpG sites can comprise a selected region of the reference genome. The first independent set of CpG sites can include at least 10, 50, 100, 500, 1000 or more CpG sites. The first independent set of CpG sites can include at most 1000, 500, 100, 50, 10 or less CpG sites. The first independent set of CpG sites can comprise 128 CpG or 256 CpG sites. The first independent set of CpG sites can be selected from a predetermined panel of CpG sites of interest. For example, of the approximately 28 million CpG sites present in the human genome, about 1.5 million can be detected by targeted methylation sequencing. The panel of 1.5 million CpG sites (e.g., the CpG sites of interest) identified by targeted methylation sequencing can be pre-determined by a targeted methylation sequencing method or selected by the practitioner based on specific experimental aims. The characterization of the human methylome by WGBS can identify CpG sites having dynamic regulatory functions or containing single nucleotide polymorphisms associated with disease compared to CpG sites that are stably methylated and have no identifiable regulatory function.

The number of CpG sites of interest can be further reduced by filtering the sequence reads using a subpanel of target sites that are of interest based on a priori knowledge. For example, CpG sites of interest can be obtained by priori knowledge identifying CpG sites or regions of the genome that are discriminative or informative in detecting cancer versus non-cancer or in differentiating between cancer types or subtypes. A proportion of the target CpG sites of interest can be further removed from the dataset using p-value filtering. Removal of CpG sites that are not included in the subpanel of CpG sites of interest can be performed during data pre-processing, or during patch design via data processing module 562 and/or data constructing module 564. Details of patch design and selection of CpG sites of interest are described elsewhere herein.

The first independent set of CpG sites can be in a CpG index of the reference genome. The CpG index of the reference genome can include a first CpG site, not present in the first independent set of CpG sites, located in the reference genome between a second CpG site and a third CpG site that are present in the first independent set of CpG sites. In other words, a patch can include noncontiguous CpG sites from the CpG index. The first independent set of CpG sites can include a first CpG site and a second CpG site that are adjacent to each other in a CpG index of the reference genome, a first fragment in the plurality of fragments can include the first CpG site but not the second CpG site, and a second fragment in the plurality of fragments can include the second CpG site but not the first CpG site. Thus, adjacent CpG sites can be present on different unique methylation sequencing fragments. Conversely, the first independent set of CpG sites can include a first CpG site and a second CpG site that are adjacent to each other in a CpG index of the reference genome, and a first fragment in the plurality of fragments can include both the first CpG site and the second CpG site. Thus, adjacent CpG sites can be present on the same unique methylation sequencing fragment. The first independent set of CpG sites can be drawn from across the entire reference genome. Each fragment in the plurality of fragments obtained by methylation sequencing can be aligned to the reference genome. Alignment to the reference genome can occur using alignment of the methylation sites (e.g., methylation pattern) in each fragment in the plurality of fragments. Alignment to the reference genome can occur using alignment of the base pairs in each fragment in the plurality of fragments (e.g., using a program such as BLAST, BLASR, BWA-MEM, DAMAPPER, NGMLR, GraphMap, Minimap, among others).

The first channel of the first patch can comprise a plurality of instances of a first plurality of parameters, where each instance of the first plurality of parameters can include a parameter for a methylation status (or methylation state) of a respective CpG site in the first independent set of CpG sites for the first patch.

Referring to FIG. 6A, a plurality of instances can comprise a plurality of parameters corresponding to each CpG site in the first independent set of CpG sites. As depicted in FIG. 6A, the first channel 532-1-1 of the first patch 530-1 comprises the plurality of instances 534-1-1-1, 534-1-1-2 to 534-1-1-M, where M is a positive integer. Moreover, in FIG. 6A, each instance can comprise L parameters 538-1-1-1-1, 538-1-1-1-2, 538-1-1-1-3, 538-1-1-1-4 . . . 538-1-1-1-L in the first instance 534-1-1-1 (where L is a positive integer), with each parameter corresponding to the L CpG sites in the first independent set of CpG sites 536-1-1-1. Similarly, FIG. 6A illustrates L parameters 538-1-1-2-1, 538-1-1-2-2, 538-1-1-2-3, 538-1-1-2-4 . . . 538-1-1-2-L in a second instance 534-1-1-2; and L parameters 538-1-1-M-1, 538-1-1-M-2, 538-1-1-M-3, 538-1-1-M-4 . . . 538-1-1-M-L in an M^{th} instance 534-1-1-M.

As illustrated in the example patch in FIG. 6A, the plurality of instances and the plurality of parameters can produce a representative 2-dimensional matrix (e.g., an image). Reframing the methylation sequencing data into a 2-dimensional matrix thus can provide a suitable input for use in convolutional neural networks. Additionally, the analysis of the dataset using convolutional neural networks can be expanded to include a plurality of parameters (e.g., characteristics or attributes) at the fragment, sample, or subject level. For example, the 2-dimensional matrix can provide local information for each respective fragment in the plurality of fragments, where between-fragment methylation state patterns can be identified either in a horizontal or vertical direction, thus identifying correlations between neighboring methylation sites or between sequence reads, respectively.

The y-axis of the 2-dimensional matrix can be increased by increasing the number of instances in the first channel of the first patch. For example, the plurality of instances of the first plurality of parameters can be between 24 and 2048. The plurality of instances of the first plurality of parameters can be 128. The plurality of instances of the first plurality of parameters can be at least 1, 10, 100, 1000, 10000 or more. In some embodiments, the plurality of instances of the first plurality of parameters can be at most 10000, 1000, 100, 10 or less. The number of instances in the plurality of instances of the first plurality of parameters can be determined based on expected read depth of the plurality of fragments plus one standard deviation across the plurality of fragments. This can be expressed as μ (read depth)+σ (std. dev.). In some such embodiments, a number of instances in the plurality of instances of the first plurality of parameters can be determined based on expected read depth of the plurality of fragments obtained from a sequencing method described elsewhere herein. For example, sequencing performed by whole genome sequencing can have an average sequencing depth of at least 1×, 2×, 3×, 4×, 5×, 6×, 7×, 8×, 9×, 10×, at least 20×, at least 30×, or at least 40× across the genome of the test subject. The sequencing depth for targeted panel sequencing can be much deeper, including but not limited to up to 1,000×, 2,000×, 3,000×, 5,000, 10,000×, 15,000×, 20,000×, or about 30,000×. The sequencing depth can be deeper than 30,000×, e.g., at least 40,000× or 50,000×.

A parameter for the methylation status in an instance of the first plurality of parameters, for a respective fragment in the plurality of fragments, can include methylated when the corresponding CpG site in the respective fragment is determined by the methylation sequencing to be methylated, unmethylated when the corresponding CpG site in the respective fragment is determined by the methylation sequencing to not be methylated, or other when the corresponding CpG site in the respective fragment is determined by the methylation sequencing to be other than methylated or unmethylated. The parameter of other can include flagged as ambiguous when the methylation sequencing fails to collectively overlap the entirety of the respective fragment, flagged as ambiguous when the underlying CpG site is not covered by paired end reads and/or when no methylation sequencing reads are found to overlap the fragment, flagged as variant when the methylation sequencing of the respective fragment finds nucleotides inconsistent with the corresponding CpG site at an expected position of the corresponding CpG site in the respective fragment, flagged as conflicted when the methylation sequencing of the respective fragment is pair-end sequencing and a methylation state of the paired end reads covering the corresponding CpG site do not report the same methylation state for the corresponding CpG site in the respective fragment, or flagged as unknown when the methylation sequencing of the respective fragment is not able to resolve the methylation state of the corresponding CpG site. Methylation states can include but are not limited to: unmethylated, methylated, ambiguous (e.g., the underlying CpG is not covered by any reads in the pair of sequence reads), variant (e.g., the read is not consistent with a CpG occurring in its expected position based on the reference sequence and can be caused by a real variant at the site or a sequence error), or conflict (e.g., when the two reads both overlap a CpG but are not consistent). Methylation states such as ambiguous, variant, and conflict can be collapsed to the ambiguous state (e.g., other). Thus, a CpG state can include three possible states, methylated, unmethylated and ambiguous.

The constructing the first patch can comprise populating, for each respective fragment in the plurality of fragments that aligns to the first independent set of CpG sites, an instance of all or a portion of the first plurality of parameters based on the methylation pattern of the respective fragment. Aligning each respective fragment in the plurality of fragments to the first independent set of CpG sites may not include that the fragment contains all the CpG sites in the first independent set of CpG sites.

The constructing of the first patch can further comprise sorting/selecting respective fragments assigned to the first patch based on their respective p-values or their starting position in the reference genome. For example, fragments can be sorted/selected prior to populating the first patch by ranking fragments by their p-value or by their starting CpG positions. Fragments can be sorted/selected by fragment length. Fragments can be populated into instances of the first patch by prioritizing fragment centering (e.g., middle-out or selecting fragments placed in the middle) or by prioritizing instance filling (e.g., top-down or selecting a couple of top-ranked fragments). The constructing of the first patch by different methods (e.g., sorting fragments by p-value or by position and/or populating instances using top-down or middle-out) can result in differences in the 2-dimensional matrix (e.g., patch). The constructing of the first patch by different methods can result in consistent classification of cancer types. For example, the populating of the first patch using any of the above embodiments or combinations thereof can provide network inputs for successful classification by generating stable patterns that are reproducible and stable across samples. FIG. 6C illustrates an example of a patch populated with methylation sequencing fragments obtained from non-cancer cfDNA, represented as a 2-dimensional matrix. Instances can be represented by the y-axis, while parameters (e.g., black color for methylated, dark gray color for unmethylated, white color for other, light gray for empty) corresponding to CpG sites can be represented by the x-axis. Fragment information can be denoted by cell shading for each pixel in the patch.

The constructing of the first patch, for a respective fragment in the plurality of fragments, can comprise i) identifying, within an instance of the first plurality of parameters of the first channel, parameters, corresponding to the CpG sites in the respective fragment, that have not previously been assigned methylation states based on another fragment in the plurality of fragments and ii) assigning for each parameter, among the identified parameters, that aligns to a corresponding CpG site of the respective fragment, the methylation state of the corresponding CpG site of the respective fragment. For example, in FIG. 6D, the identifying step can make use of any instance since no fragments have been assigned to the channel. Thus, as illustrated in FIG. 6E, a first fragment 602 can be assigned to an instance 604 of the first plurality of parameters. The first fragment can be assigned to those CpG sites within the instance 604 of the first plurality of parameters that correspond to the CpG sites of the first fragment.

More than one fragment in the plurality of fragments can be assigned to a single instance of the first plurality of parameters of the first channel in the first patch provided that more than one fragment does not have common CpG sites. Thus, continuing with the example of FIGS. 6D and 6E, a second fragment 606 can be assigned to the instance 604 of the first plurality of parameters if the second fragment CpG sites do not overlap with the CpG sites of the first fragment, as illustrated in FIG. 6F. Thus, in FIG. 6F, where a plurality of fragments are populated into a single instance, each respective fragment may not overlap any other fragment in the plurality of fragments in the instance. In this way, an instance of a plurality of parameters can be assigned more than one, more than two, more than three, more than 10, or more than 20 fragments provided that the CpG sites of the fragments do not overlap each other. When there is overlap in the CpG sites of a first and second fragments, the two fragments cannot be in the same instance of the plurality of parameters. Thus, the second fragment 606 can, instead of being assigned to instance 604 as illustrated in FIG. 6F, be assigned to instance 608 as illustrated in FIG. 6G.

In case that a number of instances of the first plurality of parameters of the first channel cannot be assigned a respective fragment, and the method 800 can further comprise zero filling parameters in instances of the plurality of parameters of the first channel that have not been assigned a fragment. For example, in FIG. 6C, a number of instances (Y-axis) cannot be assigned a respective fragment, and each of the parameters in these instances can be assigned a zero or some other nominative value.

In case that the identifying may be unable to identify, within an instance of the first plurality of parameters of the first channel, parameters corresponding to the CpG sites in the respective fragment that have not previously been assigned methylation states based on another fragment in the plurality of fragments, the method can further comprise discarding the respective fragment. Referring to FIG. 6G, all the rows of the illustrated channel can include at least one fragment whose CpG sites overlaps with the CpG sites of the respective fragment that has not yet been assigned to the channel. In such an instance, the respective fragment that has not yet been assigned to the channel can be discarded.

The number of instances in the plurality of instances in the first patch can be increased to accommodate a higher read depth. The number of instances in the plurality of instances can be up to 300, up to 500, up to 1000, up to 5000, up to 10,000 or greater than 10,000. Thus, referring to FIGS. 6D-6N, the number of rows in such embodiments can be up to 300, up to 500, up to 1000, up to 5000, up to 10,000 or greater than 10,000. A p-value threshold can be decreased (thereby lowering the number of qualifying fragments) to increase stringency of the selection of fragments and to ensure that all fragments with high signal methylation patterns are populated into the plurality of instances. As discussed in Example 8, the read depth can be altered by adjusting the hyperparameters for patch construction. As described in Example 8, the p-value can be altered by adjusting the hyperparameters for patch construction. The hyperparameter values can be determined based on the specific elements of the assay (e.g., sample size, sample type, method of methylation sequencing, fragment quality, methylation patterns, among others). The hyperparameter values can be determined using experimental optimization. The hyperparameter values can be assigned based on prior template values.

In case that the identifying is unable to identify, within an instance of the first plurality of parameters of the first channel of the first patch, parameters corresponding to the CpG sites in the respective fragment that have not previously been assigned methylation states based on another fragment in the plurality of fragments, the method can further comprise creating an additional instance of the first patch and assigning the respective fragment to the additional instance of the first patch. Thus, referring to FIG. 6D, if there is no space for the respective fragment in the patch illustrated in FIG. 6D, a new empty replica of the patch illustrated in FIG. 6D or an additional instance of the patch can be created. The method can further comprise creating 1, 2, 3, 4, 5, 6, 7, 8, 9, 10, 11, 12, 13, 14, 15, 16, 17, 18, 19, 20, or more than 20 additional patches or instances. The additional patches can comprise the same structure as the first (e.g., original) patch (e.g., FIG. 6D). Thus, the additional or duplicate patches can comprise, e.g., the same number of instances, the same set of independent CpG sites, the same number of channels, and/or the same characteristics, among others, of the original patch. The additional patches may not comprise the same structure as the first (e.g. original) patch. The additional instances can comprise the same or different structure as other instances illustrated in FIG. 6D.

The methylation pattern of a respective fragment may not include each CpG site in the first independent set of CpG sites of the first patch and the constructing the first patch, for a respective fragment in the plurality of fragments, can comprise populating parameters (e.g., assigning a numerical value to a parameter) in the instance of first plurality of parameters that correspond to CpG sites present in the respective fragment. Parameters in the instance of the first plurality of parameters can be zero filled. Thus, for example, referring to FIG. 6F, those parameters in the instance 604 that are not occupied by fragments 602 and 606 can be zero filled.

The constructing of the first patch can include that the product of the first independent set of CpG sites of the first patch and/or the number of instances in the plurality of instances of the first plurality of parameters is minimized to meet a pre-determined constraint. For example, if the first independent set of CpG sites is “100” and the number of instances in the plurality of instances of the first plurality of parameters is “50,” the product of the first independent set of CpG sites of the first patch and the number of instances in the plurality of instances of the first plurality of parameters can be 5000. The predetermined constraint can be at most 1 million, 500,000, 100,000, 50,000, 10,000, 1000, 100 or less. In some embodiments, the predetermined constraint can be at least 100, 1000, 10,000, 50,000, 100,000 or more. The constructing of the first patch can include that the first independent set of CpG sites of the first patch comprises a predetermined minimum number of CpG sites (e.g., 30 or more, 50 or more, or 100 or more) to capture higher order features across CpG sites.

The constructing of the first patch can include that the number of CpG sites in the first independent set of CpG sites of the first patch and the number of instances in the plurality of instances of the first plurality of parameters comprise the same corresponding dimensions (number of CpG sites, number of instances) as a pre-constructed matrix. The pre-constructed matrix can be a pre-trained network, such that the pre-trained network can be used to classify new inputs (e.g., new samples). In some embodiments, the pre-constructed matrix can be used as an input to the pre-trained network. The constructing of the first patch can include that the first independent set of CpG sites of the first patch is partitioned such that individual fragments in the plurality of fragments are not artificially divided during the populating of the first patch. The constructing of the first patch can include that the first independent set of CpG sites of the first patch is partitioned such that the first independent set of CpG sites in the first patch does not segment, truncate or exclude regions of high CpG site density.

After obtaining the dataset and prior to constructing the first patch, or at any stage of determining a disease/cancer condition of a test subject, the method 800 can further comprise pruning the plurality of fragments by removing from the plurality of fragments each respective fragment whose corresponding methylation pattern across the plurality of CpG sites in the respective fragment has a p-value that fails to satisfy a p-value threshold. The p-value of the respective fragment can be determined based upon a comparison of the methylation pattern of the respective fragment to a distribution of methylation patterns of the plurality of CpG sites in a plurality of reference fragments that have the plurality of CpG sites of the respective fragment. The methylation pattern of each reference fragment in the plurality of reference fragments can be obtained by a methylation sequencing of nucleic acid from biological samples obtained from a cohort of subjects that have one or more common characteristics (e.g., a cohort of healthy subjects, a cohort of healthy subjects that smoke, a cohort of subjects that do not smoke, a cohort of male subjects, a cohort of female subjects, a cohort of subjects that are above a threshold age, a cohort of subjects that are in a specified age range, a cohort of subjects that have a particular set of genetic mutations, a cohort of subjects of a particular race, etc.). This plurality of reference fragments can be obtained from a healthy cohort of subjects. The healthy cohort of subjects can comprise at least 10, 20, 50, 100, 1000 or more subjects.

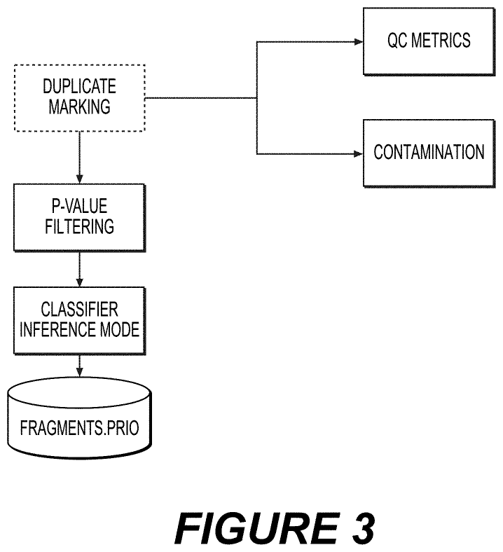

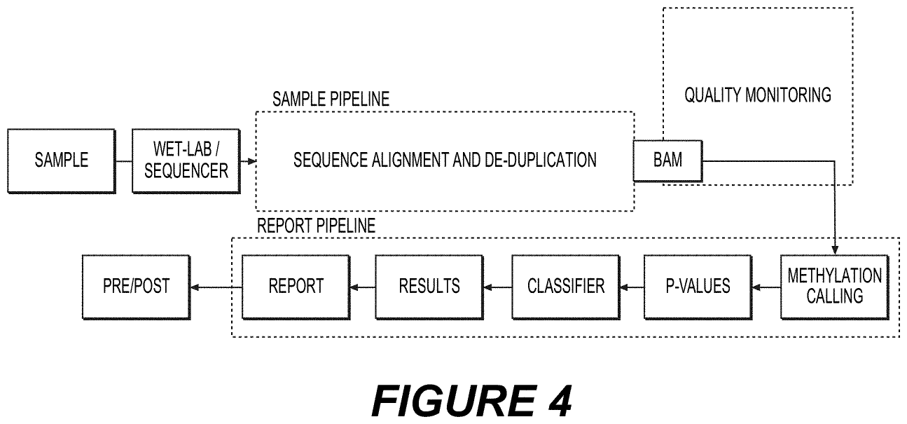

A majority of fragments obtained from blood samples of a cancer-positive patient may originate from healthy cells shedding into the bloodstream. In such cases, a subset of the plurality of fragments obtained from methylation sequencing can originate from cancer tissue. As outlined in the example workflows in FIG. 3 and FIG. 4, the p-value filter can be used to remove reads that do not have highly differential methylation statuses compared to healthy (e.g., non-cancer or “normal”) tissue. This can be performed using a generative model (e.g., a model distribution) where a cohort of healthy samples (e.g., approximately 130-150) is used to determine the normal distribution of fragment methylation patterns. The reference distribution can be generated at each locus, such that each model distribution can represent the healthy methylation status of at each locus. Based on the distribution of the reference samples, the p-value may be determined for an observed fragment, where the p-value can be the probability of observing a methylation pattern at least as unlikely as that of the observed fragment. P-values can be computed for each fragment in the plurality of fragments for each biological sample, thus providing a high-pass filter that removes low-priority or low signal methylation pattern fragments (e.g., from healthy cells) and retains those fragments of potential interest or discriminative value. The p-value threshold can be at most 0.1, 0.05, 0.01, 0.001 or less. The p-value threshold can be at least 0.0001, 0.001, 0.01, 0.05, 0.1 or more.

Referring to FIG. 6H and using the nomenclature of FIG. 6A to illustrate the first patch can comprise a plurality of channels including the first channel 532-1-1 and a second channel 532-1-2. Each channel can represent information or data associated with one characteristic (e.g., a parameter of the first characteristic). In FIG. 6A, the second channel 532-1-2 can comprise a corresponding instance of a second plurality of parameters for each instance of the first plurality of parameters of the first channel 532-1-1, where each instance of the second plurality of parameters can include a parameter for a first characteristic, other than CpG methylation state, of a respective CpG site in the first independent set of CpG sites for the first patch. The constructing of the first patch can comprise populating, for each respective fragment in the plurality of fragments (e.g., fragments 602 and 606 of FIG. 6H) that aligns to the first independent set of CpG sites, an instance of all or a portion of the first plurality of parameters and an instance of all or a portion of the second plurality of parameters based on the methylation pattern of the respective fragment. The second channel 532-1-2 can include another 2-dimensional matrix that represents an additional characteristic and/or attribute for the respective CpG site, respective fragment, respective sample, or respective subject. Thus, FIGS. 6A and 6H can illustrate a second channel 532-1-2 including a first characteristic (e.g., CpG coverage). In the exemplary embodiment of FIGS. 6A and 6H, the second channel can include a plurality of M instances (e.g., along the Y-Axis as illustrated in FIGS. 6A and 6H), where each instance comprises a plurality of parameters (each plurality illustrated as a row in FIGS. 6A and 6H) corresponding to the first independent set of L CpG sites 536-1-1-1 of the first channel 532-1-1. Then for an instance M in the plurality of instances in the second channel 532-1-2, the plurality of parameters can be indicated by 538-1-2-M-1, 538-1-2-M-2, 538-1-2-M-3, 538-1-2-M-4, and 538-1-2-M-L in FIG. 6A. Thus, fragments 602 and 606 can be aligned to the region of the genome represented by the patch illustrated in FIGS. 6A and 6H and the status of the CpG sites in the aligned fragments can be used to populate the parameters of channel 532-1-1 of the patch that correspond to these CpG sites as illustrated in FIG. 6H. For each such parameter so populated in channel 532-1-1, there can exist a corresponding parameter in the second channel 532-1-2 as illustrated in FIG. 6H. These corresponding parameters can then be populated with values associated with the additional characteristic and/or attribute for the respective CpG site, respective fragment, respective sample, or respective subject that channel 532-1-2 represents. For instance, when the additional characteristic that channel 532-1-2 is a binary representation of fragment mapping score, where, when the source fragment has a mapping score that satisfies a mapping threshold, the additional characteristic can be “1” (represented by left-leaning hash marks in FIG. 6H for purposes of illustration) and when the source fragment has a mapping score that does not satisfy the mapping threshold, the additional characteristic can be “0” (represented by right-leaning hash marks in FIG. 6H for purposes of illustration). As illustrated in FIG. 6H, fragment 606 can have a mapping score that satisfies the mapping threshold, while fragment 602 can have a mapping score that does not satisfy the mapping threshold. Note that the characteristic of channel 2 (second channel) can be a fragment-level characteristic whereas the characteristic of channel 1 (first channel) can be at the level of individual CpG sites. Thus, for channel 2, all of the parameters corresponding to a given fragment adopt the fragment level value, whereas for channel 1, each parameter representing the fragment can have a different value (the CpG methylation). This can illustrate how any given channel can sample and report, through the channel parameters, at different resolutions (e.g., at the resolution of CpG site, at the resolution of fragment, etc.).

The constructing of the first patch, for a respective fragment in the plurality of fragments, can comprise i) identifying, within an instance of the first plurality of parameters of the first channel, parameters, corresponding to the CpG sites in the respective fragment, that have not previously been assigned methylation states based on another fragment in the plurality of fragments (as discussed above with FIG. 6G), ii) assigning for each parameter, among the identified parameters, that aligns to a respective CpG site of the respective fragment, the methylation state of the respective CpG site of the respective fragment (as discussed above with FIG. 6G); and iii) assigning for each parameter, among the identified parameters, in the second plurality of parameters of the instance of the second plurality of parameters of the second channel that corresponds to the instance of the first plurality of parameters, that aligns to a respective CpG site of the respective fragment, the first characteristic of the respective CpG site of the respective fragment (as illustrated in FIG. 6H for channel 532-1-2 and as discussed above). Thus for a fragment that is populated into an instance of all or a portion of the first plurality of parameters based on the methylation pattern of the respective fragment, both the methylation state and the first characteristic of the respective CpG site other than the methylation state of the respective fragment can be populated into corresponding instances in the first and the second channels, respectively as illustrated in FIG. 6H.

More than one fragment in the plurality of fragments can be assigned to a single instance of the first plurality of parameters of the first channel in the first patch provided that the more than one fragment does not have common CpG sites, as illustrated in FIG. 6F. More than one fragment can be assigned to a single instance of the first plurality of parameters of the first channel and the second channel in the first patch provided that the more than one fragment does not have common CpG sites.

The first characteristic (e.g., the characteristic of channel 532-1-2 of FIG. 6H) of the respective CpG site can include a multiplicity of the respective fragment the respective CpG site is on. In particular, for each CpG site in the first independent set of CpG sites in the second channel of the first patch, the first characteristic can include a multiplicity that represents a number of duplicate fragments represented by the respective fragment that aligns to the respective CpG site. For example, a plurality of fragments can be considered identical multiples if they have the same start and end positions and the same methylation states at every CpG site contained in the respective fragments. In some embodiments, the multiplicity can represent a number of fragments that have at least 10%, 20%, 30%, 50%, 70%, 80%, 90% or more overlap CpG sites with each other. The multiplicity of a fragment thus can reduce the size of the input dataset while retaining valuable information. Multiple identical fragments may originate from multiple cells. In FIG. 6I, rather than the case of FIG. 6H where the characteristic of channel 532-1-2 includes fragment mapping score, the characteristic of channel 532-1-2 can include multiplicity. Further, fragment 606 can have a multiplicity of 4 whereas as fragment 602 has a multiplicity of 1. There can be four sequence reads in the biological sample that have the CpG sites of fragment 606 and one that has the CpG sites of fragment 602. Multiple identical fragments may originate from the same cell. Multiple identical fragments can include fragments that are obtained from methylation sequencing, rather than from PCR amplification, where duplicates arising from PCR amplification are removed from the dataset (e.g., de-duped) during data pre-processing. Duplicates arising from PCR amplification can be further reduced using normalization and/or enrichment steps.

The first characteristic of the respective CpG site (e.g., the characteristic of channel 532-1-2) can include a CpG β-value drawn from a healthy cohort. The β-value can be the ratio between (i) the methylated probe intensity (e.g., methylated CpG site intensity) and (ii) the sum of the methylated probe and unmethylated probe intensities. The methylated probe intensity can indicate the methylation state (e.g., a percentage of methylated sites) of a CpG site, a region, a whole genome. The methylated probe intensity can indicate the ratio of number of methylated fragments at a specific CpG site over the total number of fragments that cover the specific CpG site. Then, the β-value of the methylation state at each CpG site for a given sample can represent the number of fragments that are hypomethylated or hypermethylated as a percentage of the methylation states of the plurality of fragments at the respective CpG site. For example, a reference β-value for a respective CpG site can quantify the percentage of methylation at the CpG site in a “healthy” control or reference sample.

The first characteristic of the respective CpG site can include a CpG M-value drawn from a cohort (e.g., a cohort of healthy subjects, a cohort of healthy subjects that smoke, a cohort of subjects that do not smoke, a cohort of male subjects, a cohort of female subjects, a cohort of subjects that are above a threshold age, a cohort of subjects that are in a specified age range, a cohort of subjects that have a particular set of genetic mutations, a cohort of subjects of a particular race, etc.), a CpG M-value drawn from a predetermined tissue type in a healthy cohort, or a CpG M-value drawn from the test subject, where the M value is calculated as the log₂ ratio of the intensities of methylated probe versus unmethylated probe. See, Du et al., 2010, Comparison of Beta-value and M-value methods for quantifying methylation levels by microarray analysis,” BMC Bioinformatics. 11:587, doi:10.1186/1471-2105-11-587, which is hereby incorporated herein by reference in its entirety. Such a characteristic can be at the resolution of CpG and is illustrated in FIG. 6J. In FIG. 6J, rather than the case of FIG. 6H where the characteristic of channel 532-1-2 can be fragment mapping score, the characteristic of channel 532-1-2 can be CpG β-value or M-value drawn from a healthy cohort. Moreover, unlike FIGS. 6H and 6I, the characteristic of channel 532-1-2 may not be associated with the source of the fragments, but rather the CpG sites themselves. Therefore, channel 532-1-2 values in each column in channel 532-1-2 of FIG. 6J can have the same value since each column represents the same CpG site in the reference sequence (reference genome). That is, each column in channel 532-1-2 of FIG. 6J represents the β-value or M-value of a corresponding CpG site in the reference genome that is represented by channel 532-1-2. Rather than using a healthy cohort, a cohort of subjects having a characteristic or combination of other characteristics can be used (e.g., a cohort of healthy subjects that smoke, a cohort of subjects that do not smoke, a cohort of male subjects, a cohort of female subjects, a cohort of subjects that are above a threshold age, a cohort of subjects that are in a specified age range, a cohort of subjects that have a particular set of genetic mutations, a cohort of subjects of a particular race, etc.). The first characteristic of the respective CpG site (e.g., the characteristic of channel 532-1-2) can include a CpG β-value drawn from the test subject. This can have the result of looking exactly like FIG. 6J with the exception that the β-values can be across all the fragments of a test subject rather than those of a healthy cohort.

The first characteristic of the respective CpG site (e.g., the characteristic of channel 532-1-2) can include a Pearson's correlation score for methylation state of 5′ and 3′ neighbor CpG sites (either from a cohort or from the given subject represented). This can have the result of looking like FIG. 6J with the exception that value of a given column is a measure of correlation (e.g., a Pearson's correlation) of (i) the methylation state of the CpG in the column to the left of the given column and (ii) the methylation state of the CpG in the column to the right of the given column across all the fragments of a test subject or, alternatively, a cohort as described elsewhere herein. For instance, with reference to FIG. 6K, the characteristic of column 610 of channel 532-1-2 can correspond to a given CpG site in channel 532-1-1 (of FIG. 6J). To further illustrate, there can be ten fragments 620-1, . . . 620-10 that map to this CpG site and so there are ten CpG states to the left of the given CpG site (one for each of the ten fragments) and ten CpG states to the right of the given CpG site (one for each of the ten fragments). These ten fragments can be from the subject. The ten fragments can be from a cohort. The value being placed for the CpG site can be the Pearson's correlation score between (i) the methylation state of the ten CpG states to the left of the given CpG site (X values) and (ii) the methylation state of the ten CpG states to the right of the given CpG site (Y values). That is, (1, 0) for fragment 620-1), (0, 0) for fragment 620-2, and so forth. Computation of the Pearson's correlation score for this example using a Pearson correlation coefficient calculator can show a Pearson correlation in this example between X and Y of r(8)=0.67, p=0.34, where (8) indicates 8 degrees of freedom given 10 samples and the p value for this is 0.34. Accordingly, the entire column for the parameter 610 in channel 532-1-2 corresponding to this CpG site can be set to value 0.67 as illustrated in FIG. 6K.

Rather than a Pearson's correlation score for methylation state of 5′ and 3′ neighbor CpG sites, either from a cohort as described elsewhere herein or from the given subject represented, the characteristic can include Jaccard similarity (or Jaccard index, Jaccard similarity coefficient, and Intersection over Union) of methylation state of the respective CpG site in the test subject versus a healthy cohort. The Jaccard similarity index (or the Jaccard similarity coefficient) can compare members for two sets to see which members are shared and which are distinct. The Jaccard similarity index can be a measure of similarity for the two sets of data, with a range from 0% to 100%. The Jaccard similarity index can be the size of the intersection divided by the size of the union of the two sets of data. Thus, the example of FIG. 6K can be applicable to the Jaccard index with the exception being that the computation is that of the Jaccard similarity rather than the Person correlation. Rather than a Jaccard similarity or Pearson correlation between the left hand and right CpG sites (5′ and 3′ CpG sites), an overlap coefficient, simple matching coefficient, Sorensen-Dice coefficient, a weighted Jaccard similarity, weighted Jaccard distance, Tanimoto similarity or distance, a distance metric, or Tversky index, can be computed using the methylation state of 5′ and 3′ neighbor CpG sites, either from a cohort as described elsewhere herein or from the given subject represented.

Table 1 provides examples of the distance metrics:

TABLE 1
Example Distance Metrics

| Type | Distance Metric |
|---|---|
| Euclidean | $d ( {X}^{p} , {X}^{q} ) = \sqrt{\begin{matrix}{\sum }_{i = 1}^{n} & {( {X}_{i}^{p} - {X}_{i}^{q} )}^{2}\end{matrix}}$ |
| Manhattan distance | $d ( {X}^{p} , {X}^{q} ) = \begin{matrix}{\sum }_{i = 1}^{n} & \vert {X}_{i}^{p} - {X}_{i}^{q} \vert\end{matrix}$ |
| Maximum | d(X^{p}, X^{q}) = argmaxᵢ\|Xᵢ^{p} − Xᵢ^{q}\| |
| Value | |
| Normalized Euclidean | $d ( {X}^{p} , {X}^{q} ) = \sqrt{\frac{1}{n} {\sum }_{i = 1}^{n} {( \frac{{X}_{i}^{p} - {X}_{i}^{q}}{{max}_{i} - {min}_{i}} )}^{2}}$ |
| Normalized Manhattan | $d ( {X}^{p} , {X}^{q} ) = \frac{1}{n} {\sum }_{i = 1}^{n} \frac{\vert {X}_{i}^{p} - {X}_{i}^{q} \vert}{{max}_{i} - {min}_{i}}$ |
| Normalized Maximum Value | $d ( {X}^{p} , {X}^{q} ) = arg {max}_{i} \frac{\vert {X}_{i}^{p} - {X}_{i}^{q} \vert}{{max}_{i} - {min}_{i}}$ |
| Dice Coefficient | $d ( {X}^{p} , {X}^{q} ) = 1 - \frac{\begin{matrix}2 {Σ}_{i - 1}^{n} & {X}_{i}^{p} {X}_{i}^{q}\end{matrix}}{\begin{matrix}{Σ}_{i - 1}^{n} & \begin{matrix}{X}_{i}^{{p}^{2}} + {Σ}_{i - 1}^{n} & {X}_{i}^{{q}^{2}}\end{matrix}\end{matrix}}$ |
| Cosine distance | $d ( {X}^{p} , {X}^{q} ) = 1 - \frac{\begin{matrix}{Σ}_{i - 1}^{n} & {X}_{i}^{p} {X}_{i}^{q}\end{matrix}}{\sqrt{\begin{matrix}{Σ}_{i - 1}^{n} & \begin{matrix}{X}_{i}^{{p}^{2}} \cdot {Σ}_{i - 1}^{n} & {X}_{i}^{{q}^{2}}\end{matrix}\end{matrix}}}$ |
| Jaccard coefficient | $d ( {X}^{p} , {X}^{q} ) = 1 - \frac{\begin{matrix}{Σ}_{i - 1}^{n} & {X}_{i}^{p} {X}_{i}^{q}\end{matrix}}{\begin{matrix}{Σ}_{i - 1}^{n} & {X}_{i}^{{p}^{2}} + {Σ}_{i - 1}^{n} & {X}_{i}^{{q}^{2}} - {Σ}_{i - 1}^{n} & {X}_{i}^{p} {X}_{i}^{q}\end{matrix}}$ |

In Table 1, Xₚ=[X₁^{p}, . . . , Xₙ^{p}] and X^{q}=[X₁^{p}, . . . , Xₙ^{q}] can be two methylation state vectors, each respective element in [X₁^{p}, . . . , Xₙ^{p}] and [X₁^{p}, . . . , Xₙ^{q}] representing the methylation state of a neighboring CpG site of one of the n (where n is a positive integer) fragments mapping to the central subject CpG site as either “1” or “0,” where the values “1” and “0” represent the two possible methylation states (methylated and unmethylated) for the neighboring CpG site. For instance, each respective element in X^{p} can represent the methylation state of the 5′ neighboring CpG site in a corresponding fragment in a plurality of fragments (n fragments) mapping to the subject central CpG site whereas each respective element in X^{q} represents the methylation state of the 3′ neighboring CpG site in a corresponding fragment in the plurality of fragments mapping to the subject central CpG site. Further, maxᵢ and minᵢ can be the maximum value (“1”) and the minimum value (“0”) of an i^{th} element, respectively.

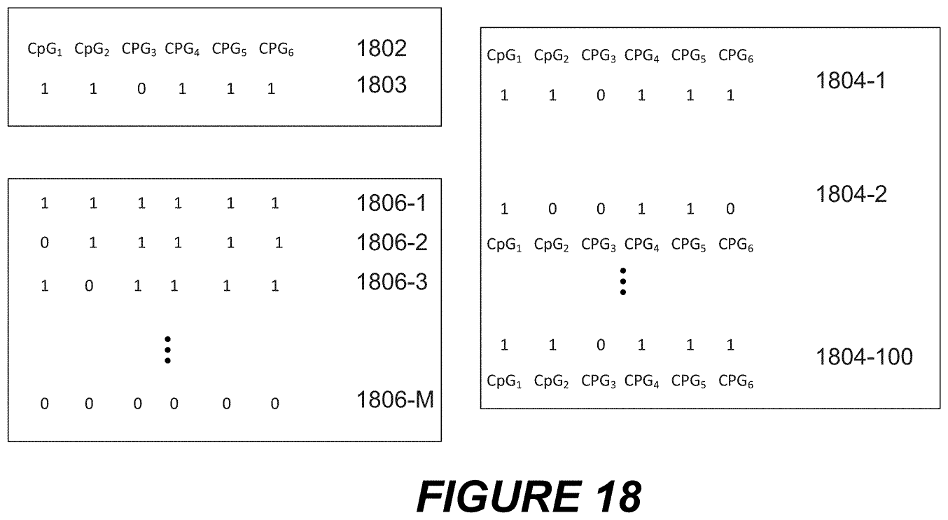

The first characteristic of the respective CpG site (e.g., the characteristic of channel 532-1-2) can include a p-value of the respective fragment. The methylation pattern of a respective fragment can be used to compute the p-value of the respective fragment in the channel as compared to those fragments in a cohort that have the same CpG sites as the respective fragment. Thus, referring to FIG. 18, if a respective fragment 1802 has six CpG sites having the hypothetical methylation pattern (1, 1, 0, 1, 1, 1), where the value “1” indicates methylated and the value “0” indicates unmethylated, then the expression “(1, 1, 0, 1, 1, 1)” can be the methylation state vector 1803 of the respective fragment 1802. In this example, the p-value for the methylation pattern of the respective fragment 1802 can be determined in relation to the methylation pattern of those fragments in a cohort that have the same six CpG sites, for instance fragments 1804-1 through 1804-100. For the respective fragment 1802, a sample probability that the respective fragment's methylation state vector 1803 occurs in comparison to the control group data 1804 can be computed by randomly sampling a subset of possible methylation state vectors 1806-1, 1806-2, 1806-3, . . . , 1806-M encompassing the CpG sites in the respective fragment's methylation state vector. As the length of the test methylation state vector 1803 is 6, there can be 2⁶ possibilities of methylation state vectors encompassing the six CpG of the fragment 1802. In a generic example, the number of possibilities of methylation state vectors can be 2″, where n is the length of the test methylation state vector. A probability corresponding to each of the sampled possible methylation state vectors 1806 can be calculated for the fragment's methylation state vector 1802 and the sampled possible methylation state vectors 1806, using for example a Markov chain model or some other form of model, thereby calculating a proportion of the sampled possible methylation state vectors 1806 corresponding to probabilities less than or equal to the probability of the methylation pattern (methylation state vector) 1803 of the respective fragment. See, for example, United States Patent Publication No. US 2019-0287652 A1, which is hereby incorporated by reference. No assumption may be made regarding the relatedness of adjacent CpG sites and thus a Markov chain model cannot be used to estimate p-value. For instance, rather than using a Markov chain model as disclosed in United States Patent Publication No. US 2019-0287652 A1, any technique for measuring statistical significance can be used as examples of which include but are not limited to moment generating functions, combinatorial methods, exponential families, asymptotic approximations, Gaussian approximations, Poisson approximations and Large Deviation approximations. An estimated p-value score for the methylation pattern 1803 of the respective fragment 1802 can then be calculated based on this calculated proportion. This p-value can represent the probability of observing the methylation state vector 1803 of the respective fragment 1802 or other methylation state vectors even less probable in the cohort that fragments 1804 are drawn from a cohort of subjects that have one or more common characteristics, as described elsewhere herein. A low p-value score, thereby, can generally correspond to a methylation state vector which is rare in the cohort, and which causes the fragment to be labeled anomalously methylated, relative to the cohort. In instances where fragments 1804 are drawn from a cohort of healthy subjects, a high p-value score for fragment 1802 can generally relate to a methylation state vector 1803 that is expected to be present, in a relative sense, in a healthy subject. If the cohort from which fragments 1804 are drawn is a non-cancerous group, for example, a low p-value for the methylation state vector 1803 can suggest that the respective fragment 1802 is anomalously methylated relative to the cohort, and therefore can be possibly indicative of the presence of cancer in the subject from which the fragment 1802 is drawn.

The first characteristic of the respective CpG site (e.g., the characteristic of channel 532-1-2) can include a length of the respective fragment the respective CpG site is on. For instance, in FIG. 6L, fragment 602 can have a length of 62 residues and fragment 606 can have a length of 98 residues. In this instance, the corresponding parameters in channel 532-1-2 for fragments 602 and 606 can be populated as illustrated, with respective values 62 and 98.

The first characteristic of the respective CpG site (e.g., the characteristic of channel 532-1-2) can include a fragment sequence source. For instance, the fragment sequence source can indicate an organ biopsied for the sequences reads of the subject. Organs can be coded in a lookup table such as “1”=brain, “2”=stomach, “3”=breast, “4”=lung, “5” blood, etc. Since it is likely that all the fragments for a given test subject are from the same organ or source, FIG. 6M can be illustrative of the situation in which fragments 602 and 606, originating from blood, are coded in channel 532-1-2. Rather than coding for source organ, the fragment sequence source can designate the type of sequencing used to obtain the sequence, e.g., “1” indicates targeted paired-end sequencing, “2” indicates targeted single-end sequencing, “3” indicates paired-end whole genome sequencing, and “4” indicates single-end whole genome sequencing, etc. The first characteristic of the channel 532-1-2 can indicate a specific method in which sequence reads were amplified and sequenced, where a lookup table can be used to track the various different possibilities, e.g., “1”=5′ transcriptome kit, “2”=3′ transcriptome kit, etc.

The first characteristic of the respective CpG site (e.g., the characteristic of channel 532-1-2) can include a fragment mapping quality score of the respective fragment. The fragment mapping quality score can be computed using the techniques of Ewing and Green, 1998, “Base-calling of automated sequencer traces using phred. ii. Error probabilities,” Genome Res. 8: 186-194. FIG. 6L can illustrate such an assignment, where fragment 606 has a mapping quality of 98 and fragment 602 has a mapping quality of 62. When multiple sequence reads contributed to the fragment (e.g., the fragment has a multiplicity of greater than 1), the fragment mapping quality score can be an average of the mapping quality scores of the multiple sequence reads.

The first characteristic of the respective CpG site (e.g., the characteristic of channel 532-1-2) can include a distance (e.g., a number of nucleotides) to a 5′ adjacent CpG site (or a distance to a 3′ adjacent CpG site) in the reference genome. In FIG. 6N, the characteristic of channel 532-1-2 can be the 5′ distance (or a distance to a 3′ adjacent CpG site) a given CpG is to its nearest neighbor CpG site. Moreover, unlike FIGS. 6H and 6I, the characteristic of channel 532-1-2 of FIG. 6N cannot be associated with the source of the fragments, but rather the CpG sites themselves. Therefore, channel 532-1-2 values in each column in channel 532-1-2 of FIG. 6N can have the same value since each column represents the same CpG site in the reference sequence (reference genome). Each column in channel 532-1-2 of FIG. 6N can represent the 5′ distance (or a distance to a 3′ adjacent CpG site) a given CpG is to its nearest neighbor CpG site. The distance can be on a linear nucleotide scale, on a logarithmic nucleotide scale, or some other function of nucleotide scale.

The first characteristic of the respective CpG site (e.g., the characteristic of channel 532-1-2) can include a genetic element the respective CpG site is within. Examples of such genetic elements can include, but are not limited to, promoter/enhancer regions, exons, introns, histone modification marks, CpG islands/shores/shelves, evolutionary conservation sites, transcription factor binding sites, restriction sites, cross-over hotspot instigator sites, and polyadenylation signals, among others. The genetic elements can be coded in a lookup table such as “1”=exons, “2”=introns, “3”=restriction sites, etc.

The first characteristic of the respective CpG site (e.g., the characteristic of channel 532-1-2) can include a biological pathway (e.g., a plurality of interactions among molecules in a cell triggered by one or more genes or biological functions that can be triggered by one or more genes) associated with the respective CpG site. The first characteristic can include a biological pathway that of the respective fragment containing the subject CpG site. Thus, when a given biological pathway comprises one or more biological functions triggered by 10 genes and if the respective fragment maps to one of these genes, then the first characteristic can be the given biological pathway. Biological pathways can be coded in a lookup table. Thus, fragment 606 of FIG. 6I can map to the biological pathway encoded in a lookup table as biological pathway “4” and fragment 602 can map to the biological pathway encoded in the lookup table as biological pathway “1.” Examples of biological pathways are found at Fabregat et al. 2018 PMID: 29145629, and Kanehisa and Goto, 2000, “KEGG: Kyoto Encyclopedia of Genes and Genomes,” Nucleic Acids Res. 28(1), pp. 27-30, each of which is hereby incorporated by reference.

The first characteristic of the respective CpG site (e.g., the characteristic of channel 532-1-2) can include a gene associated with the respective CpG site. More particularly, the first characteristic can be a gene that the respective fragment containing the subject CpG site maps to. Genes can be coded in a lookup table. Thus, fragment 606 of FIG. 6I can map to a gene encoded in a lookup table as gene “4” and fragment 602 can map to biological encoded in a lookup table as gene “1”. The first characteristic of the respective CpG site (e.g., the characteristic of channel 532-1-2) can include a value of a CpG transition impulse function for the respective CpG site. The first characteristic of the respective CpG site can include a determination as to whether the CpG site is part of a CpG island. See, Yu et al., 2017, “GaussianCpG: a Gaussian model for detection of CpG island in human genome sequences,” BMC Genomics 18(4), p. 392, which is hereby incorporated by reference for methods of determination of whether a CpG site is part of an island as well as the case in which such calculations approach an impulse function. The first characteristic of the respective CpG site (e.g., the characteristic of channel 532-1-2) can include a value of a CpG run-length encoding for the respective CpG site. See Chen et al., 2018, “Conflict of CpG density and DNA methylation are proximally and distally involved in gene regulation in human and mouse tissues,” Epgenetics 13(7), pp. 721-741, which is hereby incorporated by reference. The first characteristic of the respective CpG site can include whether or not the CpG site is in a Conflicts of Gap (COG) region, whether or not the CpG site is in a Conflict of Overlap (COO) region, whether or not the CpG site is in a Harmony with Medium Value (HMV) region, or whether or not the CpG site is in a Harmony with Extreme Value (HEV) region. See Chen et al., Id.

The first characteristic of the respective CpG site (e.g., the characteristic of channel 532-1-2) can include a read strand orientation of the fragment the respective CpG site is on. The source fragments can have a read strand orientation of R1 (5′-to-3′), R2 (3′-to-5′), or both. R1 can be represented by “1,” R2 can be represented by “2,” and both can be represented by “0.” A read strand orientation of the fragment can be in the 5′ direction or the 3′ direction. The fragment sequence source can be in the forward direction or the reverse direction.

The first characteristic of the respective CpG site can include the per fragment entropy for each respective fragment that aligns to the respective CpG site or the across-region entropy of a fixed length region comprising the respective CpG site, where the across-region entropy is calculated over all the observed methylation states that overlap the fixed length region as a group. The first characteristic of the respective CpG site can include the per-CpG site entropy for the respective CpG site, where the per-site entropy is calculated over all the instances comprising a parameter corresponding to the respective CpG site. Methods for calculating normalized methylation entropy values are disclosed in Jenkinson et al., 2017, “Potential energy landscapes identify the information-theoretic nature of the epigenome,” Nat. Genet. 49(5), pp. 719-729, which is hereby incorporated by reference.

The first characteristic of the respective CpG site can include the methylation density of a respective fragment. The methylation density can be calculated using the equation:

$$methylation density = \frac{( β - {value}_{expected healthymethylation} - β - {value}_{observed fragmentmethylation} )}{fragment base pair distance} ,$$

where β-value_{expected healthy methylation} is the β-value for the CpG site in a healthy cohort and β-value_{observed fragment methylation} is the β-value observed in the test subject for the respective CpG site. The distance to a neighboring CpG site (e.g., a 5′ adjacent or 3′ adjacent CpG site in the reference genome) (fragment base pair distance) can be between 5 to 100 base pairs away in the reference genome. The distance to a neighboring CpG site can be between 100 to 500 base pairs away, between 500 to 1000 base pairs away, between 1000 to 5000 base pairs away, between 5000 to 10,000 base pairs away, or more than 10,000 base pairs away in the reference genome. The first characteristic of the respective CpG site can be the methylation density of a fixed length region (e.g., methylation density of 100 base pairs), the minimum total coverage at the respective CpG site, or the CpG neighborhood density (e.g., CpG density in the neighboring CpG sites), where a sliding window comprising a fixed length region (e.g., a sliding window of 200 base pairs) can be used to determine the number of CpG sites in the sliding window. The first characteristic of the respective CpG site can include the methylation-weighted density, where the number of methylated CpG sites is determined for a fixed length region (e.g., a fragment or a sliding window). Details of the sliding window are described elsewhere herein. Additional method for calculating CpG methylation density are disclosed in Zhang et al., 2008 “A novel method to quantify local CpG methylation density by regional methylation elongation assay on microarray,” BMC Genomics 9(59), doi:10.1186/1471-2164-9-59, which is hereby incorporated by reference.

The first characteristic of the respective CpG site can include the genome reference position, the start or end position of the fragment in the instance of the first plurality of parameters that aligns to the respective CpG site, the length of the respective fragment the respective CpG site is on, the number of repeats in the respective fragment the respective CpG site is on, or the 5′ clipped status of the respective fragment the respective CpG site is on.

The first characteristic of the respective CpG site can include a cancer association parameter for the respective CpG site. The cancer association parameter can include any information associated with cancer. The cancer association parameter can be determined using differential methylation information, gene expression data (e.g., methylation microarrays, gene expression microarrays and/or RNA arrays or RNA sequencing), and/or genome assays. The cancer association parameter can be determined using model organism findings (e.g., research to understand human biology based on a group of research organisms such as yeast, mice, etc.). The first characteristic of the respective CpG site can be obtained or computed from an external data source such as a reference database (e.g., the Cancer Genome Atlas Program (TCGA), UCSC Genome Browser, and/or the Mouse Tumor Biology System (MTB)).

The first characteristic of the respective CpG site can include a tissue or sample-level characteristic including but not limited to tissue-of-origin, organ-of-origin, and/or replicate (e.g., to identify or adjust for batch effects and/or to detect longitudinal patterns). The first characteristic of the respective CpG site can include a subject-level or cohort-level biological prior including but not limited to smoker/non-smoker, age group, and/or gender. The first characteristic can include any attribute at the CpG site level, fragment level, sample level, tissue level, subject level or cohort level not described above that provides biological, structural, or technical context to the fragment methylation pattern.

The plurality of channels can comprise at least three channels. The third channel in the first plurality of channels can comprise a corresponding instance of a third plurality of parameters for each instance of the first plurality of parameters, where each instance of the third plurality of parameters includes a parameter for a second characteristic of a respective CpG site in the first independent set of CpG sites. The second characteristic can be other than the first characteristic but can include any of the first characteristics described in the present disclosure.

FIG. 6A illustrates an example of a plurality of channels including a third channel 532-1-3 and a fourth channel 532-1-4, each comprising a second characteristic and a third characteristic, respectively. As depicted in FIG. 6A, the third channel can include a plurality of M instances, where each instance comprises a plurality of parameters corresponding to the first independent set of L CpG sites 536-1-1-1 of the first patch 530-1. Then for an instance M in the plurality of instances in the third channel 532-1-3 of the first patch 530-1, the plurality of parameters can be indicated by 538-1-3-M-1, 538-1-3-M-2, 538-1-3-M-3, 538-1-3-M-4, and 538-1-3-M-L. Similarly, the fourth channel can include a plurality of M instances, where each instance comprises a plurality of parameters corresponding to the first independent set of L CpG sites 536-1-1-1 of the first patch 530-1. Then for an instance M in the plurality of instances in the fourth channel 532-1-4 of the first patch 530-1, the plurality of parameters can be indicated by 538-1-4-M-1, 538-1-4-M-2, 538-1-4-M-3, 538-1-4-M-4, and 538-1-4-M-L. Here the second and third characteristic can be other than the first characteristic but can include any of the first characteristics described in the present disclosure.

The plurality of channels in the first patch 530 can include at least 3, 4, 5, 6, 7, 8, 9, 10, 11, 12, 13, 14, 15, 16, 17, 18, 19, 20, or more channels 532. In some embodiments, the plurality of channels in the first patch can include at most 20, 19, 18, 17, 16, 15, 14, 13, 12, 11, 10, 9, 8, 7, 6, 5 or less channels 532. Each channel 532 in the plurality of channels in the first patch 530 can comprise a different characteristic. Two or more channels in the plurality of channels in the first patch 530 can comprise the same characteristic. The second characteristic can be any one or more of the characteristics described above for the first characteristic. One or more of the at least 3 channels in the first patch 530 can comprise any one or more of the characteristics described above for the first characteristic. FIG. 6B illustrates an example of a first patch 530-1 comprising 6 channels (e.g., methylation state, beta controls (e.g., β-value of control or healthy samples), beta sample (e.g., β-value of training or testing samples), p-value, multiplicity, and priors (e.g., biological priors associated with promoter/enhancer regions, exons, introns, histone modification marks, CpG islands, evolutionary conservation, transcription factor binding sites)). Each channel can be represented as rank 3 arrays (e.g., an array comprising 4 planes, each containing 3 rows and 5 columns) and stacked depth-wise within the first patch.

A characteristic common to a respective CpG site in the first independent set of CpG sites can, in the resulting 2-dimensional matrix that represents a respective channel of the first patch, apply to all or a portion of a column. For example, a β-value for a respective CpG site in a respective sample can be calculated using the plurality of fragments in the sample that align to the CpG site, and a β-value for a respective CpG site in a respective reference can be calculated using the plurality of fragments in the reference that align to the CpG site. As a result, the 2-dimensional matrix can appear “barcoded,” where all or a portion of a respective column of a respective channel in the first patch can be populated with the same value, as illustrated in FIG. 6N. A barcode image can be obtained for a characteristic that has a constant value for a respective CpG site, including but not limited to 5′ distance to neighboring CpG sites, 3′ distance to neighboring CpG sites, cancer association parameters, reference M-value, and/or sample M-value, among others.

A characteristic common to a respective fragment or to a region of the first independent set of CpG sites can, in the resulting 2-dimensional matrix that represents a respective channel 532 of the first patch 530, apply to all or a portion of an instance (e.g., a row), as illustrated in FIG. 6L. For example, a fragment sequence source, fragment mapping quality score, fragment p-value, fragment multiplicity, fragment position, and/or fragment length, among others, can populate all or a portion of a respective instance with the same value. A characteristic common to a respective sample, subject, or cohort can comprise a single value that applies to an entire channel of the first patch, regardless of the characteristics specific to the plurality of fragments or to the plurality of CpG sites in the first independent set of CpG sites. For example, sample-level, subject-level, or cohort-level biological priors including but not limited to smoker/non-smoker, age group and/or gender, among others, can apply the same value to the respective channel of the first patch.

Step 806 of the method 800 can comprise applying at least the first patch to a classifier thereby determining the cancer condition in the test subject. The classifier can predict cancer versus non-cancer and/or tissue-of-origin. The classifier can perform a multiclass prediction that discriminates between cancer/non-cancer/uninformative, tissue-of-origin, organ-of-origin, cancer type, and/or cancer stage.

FIG. 3 illustrates an example workflow in which a plurality of fragments filtered by p-value are applied to a classifier, in accordance with some embodiments. FIG. 3 also outlines an example in which classification is performed to discriminate cancer versus non-cancer and/or tissues of origin. Such classification can be a binary classification or a multi-class tissue-of-origin classification. Binary classification can be performed to discriminate cancer/non-cancer. Multi-class classification or any type of classifier can be performed to discriminate cancer types or subtypes from non-cancer samples including e.g., heme, non-informative samples, confounding conditions, or other unclassified samples. Where a binary cancer/no cancer classification is performed, a cutoff threshold of 0.99 or 99% specificity or above can be used for application of the classifier to a general population of samples. The cutoff specificity threshold can be greater than 70%, 80%, 85%, 90%, 95%, 98%, 99%, or 99.5%. In some embodiments, the cutoff specificity threshold can be at most 99.5%, 99%, 98%, 95%, 90% or less. A multi-class tissue-of-origin classification can be performed to discriminate between 2 to 5, 5 to 10, 10-15, 15-20, 20-30 or more than 30 different cancer types and/or subtypes. A classifier can be applied to predict an anorectal cancer, a bladder cancer, a breast cancer, a cervical cancer, a colorectal cancer, a head and neck cancer, a hepatobiliary cancer, an endometrial cancer, a kidney cancer, a leukemia, a liver cancer, a lung cancer, a lymphoid neoplasm, a melanoma, a multiple myeloma, a myeloid neoplasm, an ovary cancer, a non-Hodgkin lymphoma, a pancreatic cancer, a prostate cancer, a renal cancer, a thyroid cancer, an upper gastrointestinal tract cancer, a stage of urothelial carcinoma, or a uterine cancer. The one or more cancers can be “high-signal” cancer (defined as cancers with a greater than 50% probability of 5-year cancer-specific mortality), such as anorectal, colorectal, esophageal, head & neck, hepatobiliary, lung, ovarian, and pancreatic cancers, as well as lymphoma and multiple myeloma. High-signal cancers can be more aggressive and have an above-average cell-free nucleic acid concentration in test samples obtained from a patient. “High signal cancers” can refer to cancers that do not fall within the group of low signal cancers (e.g., uterine cancer, thyroid cancer, prostate cancer, and hormone-receptor-positive stage I/II breast cancer).

Multiple Patch Architecture.

The method can further comprise constructing a second patch comprising a corresponding first channel. This second patch can represent a second independent set of CpG sites in the reference genome of the species. Each respective CpG site in the second independent set of CpG sites can correspond to a predetermined location in the reference genome. The corresponding first channel of the second patch can comprise a corresponding plurality of instances of a first plurality of parameters. Each instance of the corresponding first plurality of parameters of the first channel of the second patch can include a parameter for a methylation status of a respective CpG site in the second independent set of CpG sites for the second patch. The disclosed systems and methods can populate, for each respective fragment in the plurality of fragments that aligns to the second independent set of CpG sites, an instance of all or a portion of the first plurality of parameters of the second patch based on the methylation pattern of the respective fragment thereby constructing the second patch. The above-described application of the first patch to a classifier can comprise applying both the first and second patches to the classifier thereby determining the cancer condition in the test subject. Some embodiments of the present disclosure can make use of three or more patches, four or more patches, 10 or more patches, 100 or more patches, or between 50 and 1000 patches, each having its own set of CpG sites and each being applied to the classifier.

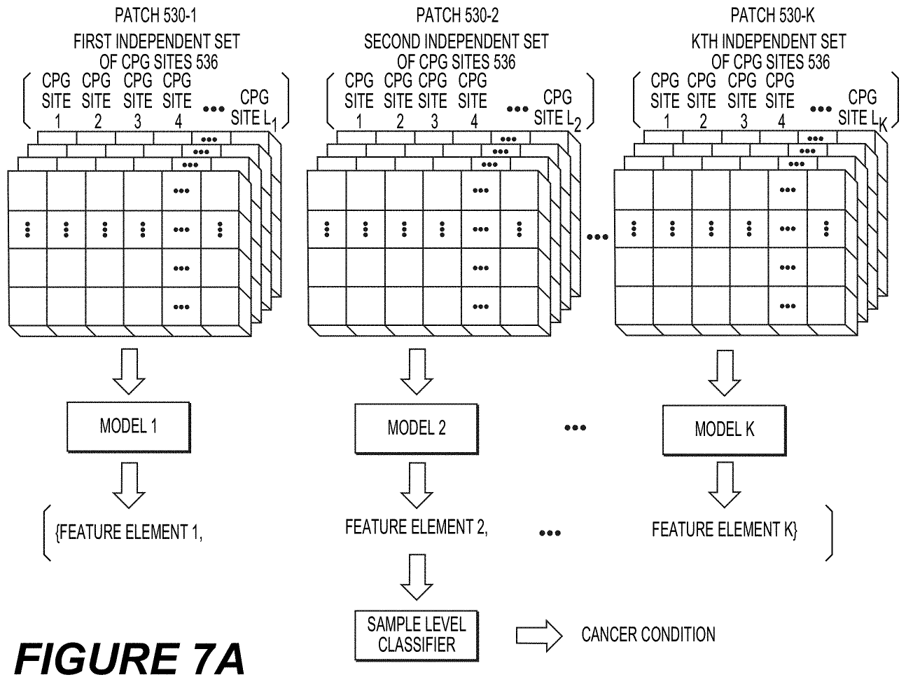

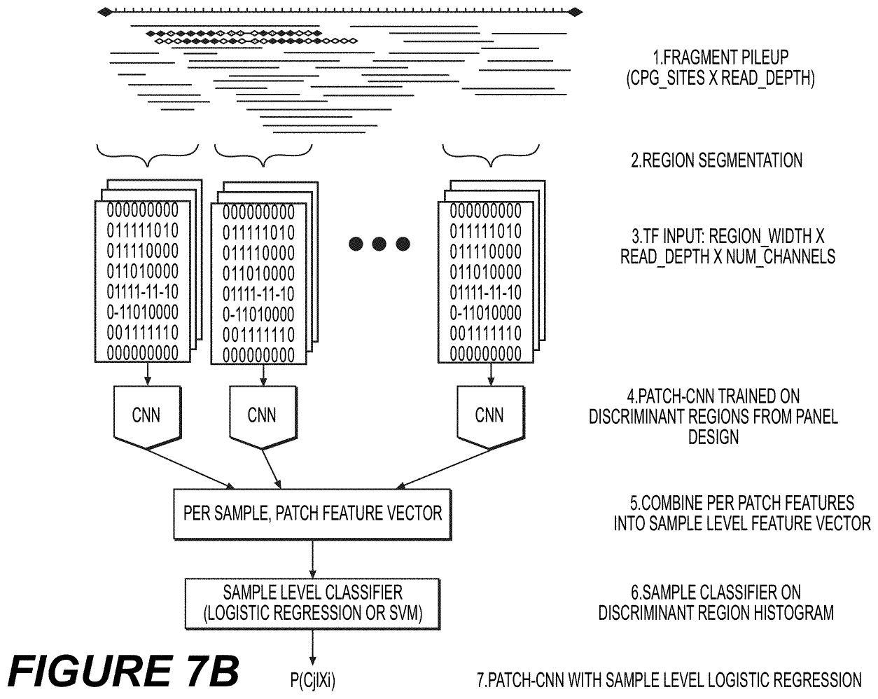

The second patch can comprise a corresponding plurality of channels including the corresponding first channel. Moreover, a corresponding second channel in the corresponding plurality of channels of the second patch can comprise a corresponding instance of a second plurality of parameters for each instance of the first plurality of parameters, where each instance of the second plurality of parameters of the second patch includes a parameter for a first characteristic, other than CpG methylation state, of a respective CpG site in the second independent set of CpG sites for the second patch. The disclosed systems and methods can further populate, for each respective fragment in the plurality of fragments that aligns to the second independent set of CpG sites, all or a portion of the instance of the second plurality of parameters of the second patch based on the methylation pattern of the respective fragment. FIGS. 7A and 7B illustrate example architectures having multiple patches, including a first patch 530-1 and a second patch 530-2, in accordance with some embodiments. The first and second independent set of CpG sites can include CpG sites 1 through L1, and CpG sites 1 through L2, respectively. Each patch can comprise a plurality of channels.

The first independent set of CpG sites may or may not overlap with the second independent set of CpG sites. The first patch can represent an equally sized, but different, portion of the reference genome than the second patch. The first patch can represent a first portion of the reference genome and the second patch represents a second portion of the reference genome, where a size of the first portion is different than a size of the second portion. For instance, the actual size in nucleotides of the first and second portion can be different. The first independent set of CpG sites can comprise a first number of CpG sites, the second independent set of CpG sites can comprise a second number of CpG sites, and the first number of CpG sites can be the same as the second number of CpG sites. In some embodiments, the first independent set of CpG sites can comprise a first number of CpG sites, the second independent set of CpG sites can comprise a second number of CpG sites, and the first number of CpG sites can be different from the second number of CpG sites.

A first patch can comprise a first number of channels and a second patch can comprise a second number of channels, where the first number and the second number of channels can be the same or different. A first patch can comprise a first number of channels comprising a first plurality of characteristics, and a second patch can comprise a second number of channels comprising a second plurality of characteristics, where the first plurality of characteristics can or cannot overlap with the second plurality of characteristics.

The disclosed systems and methods can further comprise instructions for constructing a plurality of patches. FIG. 7A illustrates an example of K patches including a first patch 530-1, a second patch 530-2, and a K^{th} patch 530-K, in accordance with some embodiments, where K is a positive integer (e.g., between 2 and 10,000) and each patch can comprise an independent set of CpG sites 536, and patch 530-K comprises a K^{th} independent set of CpG sites comprising CpG site 1 through CpG site L(K). The plurality of patches (K) can be between 1 and 10 patches, between 10 and 20 patches, between 20 and 50 patches, between 50 and 100 patches, between 100 and 500 patches, between 500 and 1000 patches, between 1000 and 5000 patches, between 5000 and 10,000 patches, or more than 10,000 patches.

The number of constructed patches in the plurality of patches can be determined by the number of CpG sites in the panel of CpG sites to be included in the classifier. The panel of CpG sites can include the entire methylome of the human genome. Thus, the number of CpG sites included across the plurality of patches can be about 28 million. The number of CpG sites included across the plurality of patches can be between 1 and 10,000, between 10,000 and 100,000, between 100,000 and 500,000, between 500,000 and 1 million, between 1 million and 1.5 million, between 1.5 million and 5 million, between 5 million and 10 million, between 10 million and 20 million, or greater than 20 million. The number of CpG sites included across the plurality of patches can be 1.5 million, the plurality of patches can comprise 5000 patches and each respective patch can comprise 300 CpG sites in the independent set of CpG sites. The number of CpG sites included across the plurality of patches can be 1.5 million, the plurality of patches can comprise 2000 patches and each respective patch can comprise 750 CpG sites in the independent set of CpG sites. The number of CpG sites included across the plurality of patches can be 1.5 million, the plurality of patches can comprise 1000 patches and each respective patch comprises 1500 CpG sites in the independent set of CpG sites. The panel of CpG sites to be included in the classifier can include redundant CpG sites.

The number of constructed patches in the plurality of patches can be determined by the computational capacity of the classifier, relative to the number of CpG sites in the independent set of CpG sites in each respective patch, the number of instances in the plurality of instances for each respective patch, and the number of channels in the plurality of channels for each respective patch. As an example, the classifier can include a VGG11 convolutional neural network, the number of constructed patches in the plurality of patches can be between 1000 and 2000, the number of CpG sites in the independent set of CpG sites for each respective patch can be 256, the number of instances in the plurality of instances for each respective patch can be 128 (e.g., a read depth of 128 fragments), and the number of channels in the plurality of channels for each respective patch can be 7. The classifier can include a residual network (e.g., ResNet) image classifier and the number of CpG sites in the independent set of CpG sites for each respective patch can be 1000.

The number of constructed patches in the plurality of patches, the number of CpG sites in the independent set of CpG sites, the number of instances in the plurality of instances, and the number of channels in the plurality of channels can be defined and or refined through the refinement of hyperparameters, as described in Example 8. The number of CpG sites included across the plurality of patches can be determined using an existing targeted methylation sequencing method or selected by the practitioner based on the experimental goals. Thus, the panel of CpG sites to be included across the plurality of patches can be further curated by identifying subregions of the panel that are highly informative and/or of high discriminative value.

Patch Design.

The methods can further comprise selecting the first independent set of CpG sites of the first patch through evaluation of a plurality of CpG methylation patterns determined by a methylation sequencing of a plurality of clinical fragments obtained from a plurality of clinical nucleic acid samples of a plurality of clinical biological samples obtained from a clinical cohort comprising a plurality of clinical subjects. The plurality of clinical subjects can include a first set of clinical subjects that have a first indication for the cancer condition and a second set of clinical subjects that have a second indication for the cancer condition. The plurality of clinical nucleic acid samples of the plurality of clinical biological samples obtained from the clinical cohort can be obtained from a study design (e.g., TCGA, CCGA). The indication for the cancer condition can include “cancer versus no cancer”. The indication for the cancer condition can include tumor of origin (e.g., “brain versus lung”). The indication for the cancer condition can include any information related to cancer, including, but not limited to, a stage of cancer, a probability of cancer, etc.

The selecting the first independent set of CpG sites can comprise determining a first ranking of a plurality of CpG sites in the reference genome based upon a respective first mutual information score (e.g., a mathematical value representing the measure of information content of a feature in distinguishing between two disease states) for a methylation status of each CpG site in the plurality of CpG sites between the first set of clinical subjects and the second set of clinical subjects. A first threshold number of CpG sites for the corresponding independent set of CpG sites for the first patch can be selected using the ranking. Thus, the mutual information can be assessed on a per-site basis, where mutual information can be a single value metric that identifies the probability mass of a first class versus a second class for a pairwise comparison at a given CpG site. For example, the mutual information score can be calculated for each respective CpG site for every pairwise comparison between the each respective pair of clinical subjects in the plurality of clinical biological samples. A high mutual information score can indicate a high level of discrimination between the paired subjects at the respective CpG site. For example, the CpG sites corresponding to the top 100, top 1000 or top 2000 mutual information scores can be selected and the remaining CpG sites cannot be selected. Any CpG site that has a mutual information score above 0.25, 0.30, 0.35, 0.40, 0.45, 0.50, 0.55, 0.60, 0.65, 0.70, 0.75, 0.80, 0.85, 0.90, 0.95, or 0.99 can be selected.

The plurality of clinical subjects can include a third set of clinical subjects that have a third indication for the cancer condition and a fourth set of clinical subjects that have a fourth indication for the cancer condition and the selecting can further comprise determining a second ranking of the plurality of CpG sites in the reference genome based upon a respective second mutual information score for a methylation status of each CpG site in the plurality of CpG sites between the third set of clinical subjects and the fourth set of clinical subjects. A second threshold number of CpG sites for the first independent set of CpG sites of the first patch can be selected using the second ranking. A respective mutual information score can be calculated between the first set of clinical subjects and the third set of clinical subjects, between the first set of clinical subjects and the fourth set of clinical subjects, between the second set of clinical subjects and the third set of clinical subjects, and/or between the second set of clinical subjects and the fourth set of clinical subjects. The plurality of clinical subjects can include 5 or more, 10 or more, 50 or more, 100 or more, 500 or more, 1000 or more, 2000 or more, 5000 or more, 10,000 or more, or 20,000 or more sets of clinical subjects, where each set of clinical subjects has a corresponding indication for the cancer condition.

The ranking of the plurality of CpG sites in the reference genome based on a first or second mutual information score can be performed by ranking CpG sites from highest to lowest mutual information score. The first and/or second threshold number of CpG sites for the first independent set of CpG sites of the first patch can be selected using the top-ranked mutual information scores for the plurality of CpG sites (e.g., CpG sites having the highest mutual information scores regardless of the cancer conditions used in the comparison). The first and/or second threshold number of CpG sites for the first independent set of CpG sites of the first patch can be selected from the top-ranked mutual information scores of each respective pair of clinical subjects for which a mutual information score is calculated (e.g., CpG sites having the highest mutual information scores such that all pairwise comparisons are represented in the selected set of CpG sites). The top 1000 high mutual information CpG sites can be selected for each respective pair of clinical subjects in the plurality of pairwise comparisons based on the ranking of the mutual information scores. A mutual information score for a respective CpG site can be considered discriminative for multiple pairwise comparisons of clinical subjects.

The plurality of CpG sites with the highest ranking mutual information scores can be selected as the first independent set of CpG sites of the first patch, and the first independent set of CpG sites can be arranged in the first patch in order of highest to lowest mutual information score. The first independent set of CpG sites can be arranged in the first patch in order of lowest to highest mutual information score. The patches can comprise 256 CpG sites with top-ranking mutual information scores. The constructing of the first patch can further comprise sorting respective fragments assigned to the first patch based on their respective first mutual information score. For example, prior to the constructing of the first patch, fragments can be ranked based on their respective mutual information score and populated into instances of the first patch in the order of their respective mutual information score (e.g., highest to lowest, or lowest to highest).

The first indication for the cancer condition can be a first cancer type and the second indication for the cancer condition can be a second cancer type. The first cancer type or the second cancer type can be any cancer described elsewhere herein. Then, the plurality of pairwise comparisons between the clinical subjects can include any possible pairwise comparison between any two cancer types (e.g., breast versus lung cancer).

Each respective CpG site in the first threshold number of CpG sites for the first independent set of CpG sites of the first patch can be padded in the reference genome from all other CpG sites in the first threshold number of CpG sites by a threshold number of residues. For example, each CpG site can be padded by at least 10, 20, 30, 40, 50, 60, 70, 80, 90, 100, 200, or 300 residues in order to be included in the patch. The selecting of the first independent set of CpG sites can be performed using a plurality of clinical nucleic acid samples from a plurality of clinical biological samples that is set aside for patch design (e.g., a reference database or pilot study). For example, a first set of samples can be used to select CpG sites of interest for patch design, and a second set of samples can be used to populate the respective instances of the respective patches for classification.

The CpG selecting step of the methods can further comprise determining a first ranking of a plurality of fixed length regions in the reference genome based upon a respective first mutual information score for a methylation status of a CpG site methylation pattern of each fixed length region in the plurality of fixed length regions between the first set of clinical subjects and the second set of clinical subjects. Then, a first threshold number of CpG sites can be selected for the first independent set of CpG sites of the first patch from those fixed length regions in the plurality of fixed length regions using the first ranking. Thus, a high mutual information score can indicate a high level of discrimination between the paired subjects at the fixed length region. A mutual information score for a fixed length region can be calculated using a mixture model. See, for example, United States Patent Publication No. US 2020-0365229 A1, entitled “Model-Based Featurization and Classification,” which is hereby incorporated by reference. The mixture model can be a probabilistic model for representing the presence of subpopulations within an overall population. The fixed length regions can be obtained using an external database or reference panel of probes (e.g., select regions obtained using a plurality of probes in a targeted sequencing assay to identify regions of interest from which to obtain CpG sites of interest). The fixed length regions can be obtained using a fixed length “sliding window” that slides across the entire genome or across a reference panel.

For example, a first independent set of CpG sites can be selected by sliding window (a window of 100, 200, 300, 400, 500, 600, 700, 800, 900, 1000, or 2000 base pair (bp)) across genomic regions (e.g., genomic regions corresponding to probes in a targeted sequencing assay) in a pairwise comparison between two clinical biological samples obtained from two clinical subjects. For each frame of the sliding window, a mutual information score can be calculated using a statistical model (e.g., mixture model) of the CpG sites within the respective frame of the sliding window. A mutual information score can denote the probability of the methylation pattern for a first cancer condition versus a second cancer condition at the respective region in the respective frame of the sliding window, thus indicating the discriminative power of the respective region. A mutual information score can be similarly calculated for each region in each frame of the sliding window as it progresses across the select genomic regions.

The length of the sliding window can be less than 10, between 10 and 50, between 50 and 100, between 100 and 200, between 200 and 500, between 500 and 1000, between 1000 and 2000, between 2000 and 5000, or greater than 5000 bp long. The sliding window can be 256 bp long. The fixed-length region of the sliding window can comprise less than 5 CpG sites, between 5 and 10 CpG sites, between 10 and 20 CpG sites, between 20 and 50 CpG sites, between 50 and 100 CpG sites, between 100 and 200 CpG sites, between 200 and 500 CpG sites, or greater than 500 CpG sites.

A first ranking of a plurality of fixed length regions (windows) can be performed by ranking the fixed length regions in order of mutual information scores from highest to lowest, or from lowest to highest. The fixed length regions can comprise one or more CpG sites, and the first independent set of CpG sites can comprise CpG sites that are obtained from top-ranking mutual information fixed length regions. The first independent set of CpG sites can comprise top-ranking mutual information fixed length regions.

The plurality of clinical subjects can include a third set of clinical subjects that have a third indication for the cancer condition and a fourth set of clinical subjects that have a fourth indication for the cancer condition and the selecting can further comprise determining a second ranking of the plurality of fixed length regions in the reference genome based upon a respective second mutual information score for a methylation status of a CpG site methylation pattern of each fixed length region in the plurality of fixed length regions between the third set of clinical subjects and the fourth set of clinical subjects; and selecting a second threshold number of CpG sites for the first independent set of CpG sites of the first patch using the second ranking.

A respective mutual information score for a fixed length region can be calculated between the first set of clinical subjects and the third set of clinical subjects, between the first set of clinical subjects and the fourth set of clinical subjects, between the second set of clinical subjects and the third set of clinical subjects, and/or between the second set of clinical subjects and the fourth set of clinical subjects. The plurality of clinical subjects can include 5 or more, 10 or more, 50 or more, 100 or more, 500 or more, 1000 or more, 2000 or more, 5000 or more, 10,000 or more, or 20,000 or more sets of clinical subjects, where each set of clinical subjects has a corresponding indication for the cancer condition.

The first and/or second threshold number of CpG sites for the first independent set of CpG sites of the first patch can be selected using the top-ranked mutual information fixed length regions in the plurality of fixed length region (e.g., CpG sites obtained from fixed length regions having the highest mutual information scores regardless of the cancer conditions used in the comparison). The first and/or second threshold number of CpG sites for the first independent set of CpG sites of the first patch can be selected using the top-ranked mutual information fixed length regions of each respective pair of clinical subjects for which a mutual information score is calculated (e.g., fixed length regions having the highest mutual information scores such that all pairwise comparisons are represented in the selected set of CpG sites). The top 100, 200, 300, 400, 500, 600, 700, 800, 900, 1000, or 2000 mutual information fixed length regions can be selected for each respective pair of clinical subjects in the plurality of pairwise comparisons based on the ranking of the mutual information scores. A mutual information score for a respective fixed length region can be considered discriminative for multiple pairwise comparisons of clinical subjects.

The constructing of the first patch can further comprise sorting respective fragments assigned to the first patch based on their respective first mutual information score (e.g., fixed length regions are sorted by lowest to highest mutual information score or by highest to lowest mutual information score). The first independent set of CpG sites in the first patch can comprise fixed length regions and/or CpG sites obtained from fixed length regions, arranged in order of mutual information scores (e.g., lowest to highest or highest to lowest). The first indication for the cancer condition can be a first cancer type and the second indication for the cancer condition can be a second cancer type. Then, the plurality of pairwise comparisons between the clinical subjects can be any possible pairwise comparison between any two cancer types (e.g., breast versus lung cancer).

Each respective CpG site in the first threshold number of CpG sites for the first independent set of CpG sites of the first patch can be padded in the reference genome from all other CpG sites in the first threshold number of CpG sites by a threshold number of residues (e.g., each CpG site obtained from a fixed length region can be padded by at least 10, 20, 30, 40, 50, 60, 70, 80, 90, 100 or 200 residues in order to be included in the patch). The plurality of fragments can be obtained using an array-based methylation sequencing, and the first ranking of a plurality of CpG sites in the reference genome for a methylation status of each CpG site in the plurality of CpG sites between the first set of clinical subjects and the second set of clinical subjects can be based upon a β-value or an M-value.

The selection of a first independent set of CpG sites for a first patch through evaluation of a plurality of CpG methylation patterns can further comprise selecting a first independent set of CpG sites for a first patch and selecting a second independent set of CpG sites for a second patch. The selection of a first independent set of CpG sites for a first patch through evaluation of a plurality of CpG methylation patterns can further comprise selecting a respective independent set of CpG sites for a respective patch in a plurality of patches.

Classifier Prediction and Training.

The methods can further comprise instructions for constructing a plurality of patches including the first patch, each respective patch being for a different independent set of CpG sites in the reference genome. The constructing the first patch can construct a plurality of patches including the first patch. The above-described classifier can comprise one or more first stage models and a second stage model. The first stage model can be a pre-trained (or trained) model. Further, the above-disclosed application of the at least first patch to a classifier can comprise obtaining a feature vector comprising a plurality of feature elements, where each feature element in the plurality of feature elements is an output of a corresponding first stage model in the one or more first stage models upon application of a respective patch in the plurality of patches to the corresponding first stage model (wherein each of the patches can be, for example, formed from data acquired from methylated nucleic acid fragments from a test subject). Application of the at least first patch to a classifier can further comprise applying the feature vector to the second stage model thereby determining the cancer condition in the test subject.

The plurality of patches can be between 10 patches and 10000 patches, or between 100 patches and 3000 patches. FIG. 7A illustrates a set of K patches, where the plurality of trained first stage models comprises Trained Model 1, Trained Model 2, through Trained Model K, where K is a positive integer (e.g., between 2 and 3000) in accordance with some embodiments. The first stage model can include a patch level classifier and the second stage model can include a sample level classifier. The application of the feature vector to the second stage model can determine whether the test subject is cancer or non-cancer, or identifies a tissue-of-origin, organ-of-origin, cancer type, and/or cancer stage. The application of the feature vector to the second stage model can be performed in a responsive manner such that patches that are positively classified in the first stage model (e.g., cancer-positive) are applied to the second level classifier. Although FIG. 7A illustrates K trained models, in some other embodiments, the set of K patches can be input data for one model instead of K trained models. The one model can be either trained or untrained. In this situation, the one model can be further trained with K patches, either sequentially or parallelly, if the K patches are obtained from training samples. In another situation, the one trained model can be used to determine a cancer condition or produce data for further analysis by the second stage model (e.g., a sample level classifier) based on the K patches, if the K patches are obtained from testing sample.

Each respective first stage model in the one or more first stage models can include a corresponding convolutional neural network, and the first channel of the first patch can include two dimensional with each respective instance of the plurality of instances of the first plurality of parameters of the first patch forming a first dimension and the first plurality of parameters of the first patch forming the second dimension (e.g., as illustrated for patch 530-1 in FIG. 7A). The second stage model can include a logistic regression model. See, for example, United States Patent Publication No. US 2019-0287652 A1, entitled “Anomalous Fragment Detection and Classification,” which is hereby incorporated by reference. The second stage model can include a support vector machine. When used for classification, SVMs can separate a given set of binary labeled data training set with a hyper-plane that is maximally distant from the labeled data. For cases in which no linear separation is possible, SVMs can work in combination with the technique of “kernels,” which automatically realizes a non-linear mapping to a feature space. The hyper-plane found by the SVM in feature space can correspond to a non-linear decision boundary in the input space. The second stage model can include any machine learning models or statistical models (e.g., decision tree models, random forest models, Naïve Bayes, K-Nearest Neighbors, Stochastic Gradient Descent) that can perform classification based on any data or information disclosed herein.

The classifier can comprise a plurality of first stage models (e.g., trained/untrained models of FIG. 7A) and a dynamic neural network (e.g., sample level classifier of FIG. 7A). The methods can further comprise constructing a plurality of patches including the first patch, each respective patch being for a different set of CpG sites in the reference genome. The constructing the first patch can comprise constructing a respective patch including the first patch. The application of the at least first patch to a classifier can comprise applying each respective patch in the plurality of patches to a corresponding first stage model in the plurality of first stage models. The corresponding first stage model can comprise i) a respective input layer for receiving the respective patch, where the respective patch comprises a first number of dimensions; ii) a respective fully connected embedding layer that comprises a corresponding set of weights, where the respective fully connected embedding layer directly or indirectly receives output of the respective input layer, and where a respective output of the respective embedding layer is a second number of dimensions that is less than the first number of dimensions; and iii) a respective output layer that directly or indirectly receives output from the respective fully connected embedding layer. The corresponding first stage model can further comprise one or more convolutional layers. The one or more convolutional layers can be placed between the respective input layer and the respective fully connected embedding layer. The one or more convolutional layers can comprise at least 1, 2, 3, 4, 5, or more layers. In some embodiments, the one or more convolutional layers can comprise at most 5, 4, 3, 2 or less layers. For multiple convolutional layers in the first stage model, neurons of a first convolutional layer connected to the respective input layer may not be connected to every single pixel in the respective patch (e.g., an input 2-dimensional image) received by the respective input layer. Similarly, neurons of a second convolutional layer may not be connected to every single neuron of the first convolutional layer. In this situation, the size of the first convolutional layer can be smaller than the size of the respective input layer, and/or the size of the second convolutional layer can be smaller than the size of the first convolutional layer. The application of the at least first patch to a classifier can further comprise inputting an aggregate of the respective output from each respective fully connected embedding layer of each trained first stage model in the plurality of first stage models into the dynamic neural network (e.g., a sample level classifier) thereby determining the cancer condition in the test subject. Each respective fully connected embedding layer can represent a set of values (e.g., scores) for each respective patch (e.g., region), and the set of scores per region can indicate the embedding size.

The respective output of the respective embedding layer of each respective first stage model in the plurality of first stage models can be a set of between 32 and 1048 values. The respective output of the respective embedding layer of each respective first stage model in the plurality of first stage models can be 128.

The aggregate of the respective output from each respective fully connected embedding layer of each trained first stage model in the plurality of first stage models can be a concatenation of the respective scores for each respective patch. For example, FIG. 7B illustrates an example of a classifier, where the classifier is a patch convolutional neural net (Patch CNN) with two-step classification performed using fragments from methylation sequencing. Each respective first stage model can include a patch level feature extractor that outputs a corresponding element into a feature vector comprising the respective patch features for each respective patch, and the sample level classifier can include a logistic regression model or a support vector machine. The application of the at least first patch to the classifier can comprise applying a plurality of patches comprising a plurality of channels to the classifier, each respective patch in the plurality of patches inputted into a corresponding first stage model (e.g., a corresponding CNN of FIG. 7B).

The classifier can comprise one first stage model and a machine learning/statistical model (e.g., a dynamic neural network or a sample level classifier of FIG. 7A). The methods can further comprise constructing a plurality of patches including the first patch, each respective patch being for a different set of CpG sites in the reference genome. The constructing the first patch can comprise constructing a respective patch including the first patch. The application of the plurality of patches to a classifier can comprise applying the plurality of patches to a first stage model (e.g., a convolutional neural network). In this situation, the first stage model can comprise i) an input layer for receiving the plurality of patches, either sequentially or parallelly, where a first patch of the plurality of patches comprises a first number of dimensions; ii) a fully connected embedding layer that comprises a set of weights, where the fully connected embedding layer directly or indirectly receives output of the input layer, and where an output of the embedding layer comprises a second number of dimensions that is less than the first number of dimensions; and iii) an output layer that directly or indirectly receives output from the fully connected embedding layer. The first stage model can further comprise one or more convolutional layers. The one or more convolutional layers can be placed between the input layer and the fully connected embedding layer. The one or more convolutional layers can comprise at least 1, 2, 3, 4, 5, or more layers. In some embodiments, the one or more convolutional layers can comprise at most 5, 4, 3, 2 or less layers. For multiple convolutional layers in the first stage model, neurons of a first convolutional layer connected to the input layer may not be connected to every single pixel in the patch (e.g., an input 2-dimensional image) received by the input layer. Similarly, neurons of a second convolutional layer may not be connected to every single neuron of the first convolutional layer. In this situation, the size of the first convolutional layer can be smaller than the size of the input layer, and/or the size of the second convolutional layer can be smaller than the size of the first convolutional layer. The application of the plurality of patches to a classifier can further comprise inputting the output from the fully connected embedding layer into the machine learning/statistical model thereby determining the cancer condition in the test subject. The fully connected embedding layer can represent a set of values (e.g., scores) for each patch (e.g., region), and the set of scores per region can indicate the embedding size.

The classifier can comprise a plurality of first stage models and a machine learning/statistical model (e.g., a dynamic neural network or a sample level classifier of FIG. 7A), where the number of the plurality of the first stage models is less than the number of one or more patches. For instance, the classifier can comprise two first stage models (e.g., two convolutional neural networks) and the number of patches can be 1000. In this situation, a portion of the 1000 patches (e.g., 400 patches) can be input data to one of the two first stage models, and the rest of the 1000 patches (e.g., 600 patches can be input data to the other one of the two first stage models.

The methods can further comprise training the one or more first stage models (e.g., CNN models of FIG. 7B) and the dynamic neural network (e.g., sample level classifier of FIG. 7B) using a cohort of subjects, where the cohort of subjects comprises a first subset of subjects that have a first label for the cancer condition and a second subset of subjects that have a second label for the cancer condition. The training can comprise a) stratifying, on a random basis, the cohort of subjects into a plurality of groups based on any combination of cancer condition, age, smoking status, or sex; b) using a first group in the plurality of groups as a training group and the remainder of the plurality of groups as test/validation groups to train the one or more first stage models (e.g., CNN models of FIG. 7B) and the dynamic neural network (e.g., sample level classifier of FIG. 7B) against the training group; c) repeating the using b) for each group in the plurality of groups so that each group in the plurality of groups is used as the training group in an iteration of the using b); and d) repeating the stratifying a), using b) and repeating c) until a classifier performance criterion is satisfied. The training group can comprise at least 10%, 20%, 30%, 40%, 50%, 60%, 70%, 80%, 90% or more of the information or data obtained from the cohort of subjects. In this situation, the test group can comprise at most 90%, 80%, 70%, 60%, 50%, 40%, 30%, 20%, 10% or less of the information or data obtained from the cohort of subjects. In some embodiments, the training group can comprise at most 90%, 80%, 70%, 60%, 50%, 40%, 30%, 20%, 10% or less of the information or data obtained from the cohort of subjects. In this situation, the test group can comprise at least 10%, 20%, 30%, 40%, 50%, 60%, 70%, 80%, 90% or more of the information or data obtained from the cohort of subjects. The classifier performance can be about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 98.5, 99, 99.5, 99.6, 99.7, 99.8, or 99.9 percent sensitivity (accuracy) at about 80, 80, 81, 82, 83, 84, 85, 86, 87, 88, 89, 90, 91, 92, 93, 94, 95, 96, 97, 98, 98.5, 99, 99.1, 99.2, 99.3, 99.4, 99.5, 99.6, 99.7, 99.8, or 99.9 percent specificity across the cohort of subjects.

For example, a classifier can be trained by obtaining patient samples (e.g., for a cohort of subjects), where each such patient is labeled with their cancer condition and using the methylation data for such subjects to populate a plurality of patches (e.g., using a method for patch design such as mutual information, prior knowledge, hyperparameters, and/or pre-existing models, among others). For each respective sample that populates a respective patch, the cancer condition indicator can be assigned to the patch for patch-level classifier training against the patient labels (e.g., training a plurality of first stage models).

For a classifier comprising a plurality of first stage models, each first stage model (e.g., patch-level convolutional network) can be trained as a binary classifier and used as a feature extractor, and the output of each respective first stage model (e.g., patch-level convolutional network) can be an intermediate feature vector concatenated across the plurality of regions corresponding to the plurality of first stage models. Each such intermediate vector corresponds to a different patient in the cohort. The output of each respective first stage model can include a plurality of activations (e.g., outputs of rectified linear units (ReLU), tanh, sigmoid, etc.) from an intermediate fully connected classification layer within the respective first stage model. The activations from each respective first stage model (responsive to input of a corresponding patch) can be used to generate a respective overall score or a vector of embeddings for each of the subjects. A sample level classifier, for instance in the form of a deep-and-wide deep neural net (DNN) classifier, can be trained on the respective overall score or the vector of embeddings and the respective label of each of the subjects.

The above described training of the plurality of first stage models (e.g., CNNs) and sample level classifier (e.g., dynamic neural network) can comprise a 3×6-fold cross-validation. Cross-validation can comprise splitting the training dataset into a smaller training dataset and a validation dataset, then training the first stage model against the smaller training set and evaluating the first stage models against the validation dataset. For instance, the training dataset can be subdivided into 6 bins equally stratified by all classifications and/or biological priors of interest (e.g., cancer/non-cancer, cancer type, cancer stage, age, and/or smoking status, among others), such that each training bin can be as uniform as possible. Training can be performed (e.g., as described above) using 5 of the six bins, with validation performed with the 6^{th} bin (cross validation). This process can be repeated six times such that each of the six bins is used once for validation. The training dataset can be randomized and shuffled three times, and the stratification, training, and validation can be repeated such that a total of eighteen training runs is performed. The classifier performance criterion can be a three-fold randomization of the dataset. Both the first stage model and the second stage model can be trained during each respective fold of 3×6-fold cross-validation. Rather than using 3×6-fold cross-validation, P×Q-fold cross validation can be used, where P and Q are positive integers and may be the same or different. The training dataset can be subdivided into P bins equally stratified by all classifications and/or biological priors of interest (e.g., cancer/non-cancer, cancer type, cancer stage, age, and/or smoking status, among others), such that each training bin can be as uniform as possible. Training can be performed (e.g., as described above) using P-1 of the P bins, with validation performed with the P^{th} bin. This process can be repeated Q times such that each of the P bins can be used once for validation. The training dataset can be randomized and shuffled P times, and the stratification, training, and validation can be repeated such that a total of P×Q training runs is performed. P can be at least 2, 3, 4, 5, 6, 7, 8, 9, 10, 11, 12, 13, 14, 15, 16, 17, 18, 19, 20 or more. Q can be at least 2, 3, 4, 5, 6, 7, 8, 9, 10, 11, 12, 13, 14, 15, 16, 17, 18, 19, 20 or more.

The cancer condition can include tissue of origin (or tissue-of-origin, TOO) and each subject in the cohort of subjects is labeled with a tissue of origin. The cohort can include subjects that have any type of cancer or a combination of cancers described elsewhere herein. The cancer condition can include a stage of a specified cancer and each subject in the cohort of subjects is labeled with a stage of a specified cancer. The cohort can include subjects that have a stage of any type of cancer or a combination of cancers described elsewhere herein. The cancer condition can include whether or not a subject has cancer and the stratifying a) ensures that each group in the plurality groups has equal numbers of subjects that have cancer and that do not have cancer.

The number of trainable parameters of a classifier of the present disclosure can be scaled to a respective dataset during training (e.g., VGGNet: 140 million trainable parameters versus Patch-CNN 16: 345,000 trainable parameters). Dropout can be applied to control overfitting and improve classification of small training sets by creating a learned weighted ensemble and reducing the network complexity. Up to 50% dropout can be applied. The training can eliminate one or more patches in the plurality of patches using L1 regularization (e.g., Lasso regression) or L2 regularization (Ridge regression) based upon values provided by the respective output layer of each respective patch in the plurality of patches during the training. L2 regularization can be used with coefficients up to 10% and hypertuned batch size. Training can eliminate one or more patches in the plurality of patches using early stopping with a limited number of epochs and/or metric-based early stopping. Training can be performed using aggressive dropout at 0.5, L1 regularization, decaying learning rate, Adam optimizer and large batch size at 256. Training can be performed using a slanted triangular learning rate rather than a decaying learning rate.

A feature vector obtained from a binary classifier trained on cancer/non-cancer can be used to train a multi-class classifier for tissue-of-origin, organ-of-origin, cancer type and/or cancer stage. Transfer learning from a cancer/non-cancer classifier to a multi-class (e.g., tissue-of-origin) classifier can result in an increase in accuracy in the tissue of origin classifier. See U.S. Provisional Patent Application No. 62/851,486, entitled “Systems and Methods for Determining Whether a Subject has A Cancer Condition Using Transfer Learning,” filed May 22, 2019, which is hereby incorporated by reference for disclosure on such transfer learning. The increase in accuracy in the multi-class classifier can be greater than 1%, greater than 5%, greater than 10%, greater than 15%, greater than 20%, or greater than 50%.

The classifier can comprise a patch CNN classifier that comprises one or more CNN classifiers (e.g., one for each patch as illustrated in FIG. 7B) followed by a sample level classifier that performs average-pooling, max-pooling, aggregation of patches by 3-norm pooling, logistic regression with or without Gaussian smoothing, or -means modeling on extracted features from the plurality of CNN classifiers. The classifier can comprise a patch CNN classifier that comprises one or more CNN classifiers (e.g., one for each patch as illustrated in FIG. 7B). Each such CNN can use a pre-trained CNN model. The pre-trained CNN model can use one or more layers of a convolutional neural net that has been trained on pixelated image data (e.g., RGB pixelated images). Examples of such pre-trained CNN model can include, but are not limited to, LeNet, AlexNet, VGG11, VGGNet 16, GoogLeNet, or ResNet. The pre-trained CNN model can comprise a multilayer neural net, a deep convolutional neural net, a visual geometry convolutional neural net, or a combination thereof. The pre-trained CNN model can comprise all the layers of a convolutional neural network that has been trained on non-biological data, other than the classification layers of the convolutional neural network. The pre-trained CNN model can be a 16-layer pre-trained CNN model. The sample level classifier can comprise a pre-trained 16-layer CNN model.

An example network architecture for a first level classifier is detailed below in Table 2, for a customized VGG-11 convolutional neural network architecture with two fully connected layers and softmax output layer. Traditional VGG-11 can comprise a convolutional filter size of 3×3 and use ReLU activation function. For this customized VGG-11 CNN, convolutional filter (e.g., convolution kernels) shapes can be adjusted to 1×3 to capture intra-fragment sequences over fragment pileups with two-dimensional convolution of matrices (Conv2d), and leaky rectified linear unit activation (ReLU) activation function can be used in-place of ReLU.

TABLE 2
Network architecture for a customized
VGG-11 convolutional neural network

| | Filter Size, | Output Width × |
|---|---|---|
| Network Layers | Stride, Filters | Height × Channel |
| Input | —, —, — | 224 × 32 × 20 |
| Conv2d + Leaky ReLU | 1 × 3, 1, 64 | 224 × 32 × 64 |
| Conv2d + Leaky ReLU | 1 × 3, 1, 64 | 224 × 32 × 64 |
| Max Pooling | 2 × 2 | 112 × 16 × 64 |
| Conv2d + Leaky ReLU | 1 × 3, 1, 128 | 112 × 16 × 128 |
| Conv2d + Leaky ReLU | 1 × 3, 1, 128 | 112 × 16 × 128 |
| Max Pooling | 2 × 2 | 64 × 8 × 128 |
| Conv2d + Leaky ReLU | 1 × 3, 1, 256 | 64 × 8 × 256 |
| Conv2d + Leaky ReLU | 1 × 3, 1, 256 | 64 × 8 × 256 |
| Max Pooling | 2 × 2 | 32 × 4 × 256 |
| Conv2d + Leaky ReLU | 1 × 3, 1, 512 | 32 × 4 × 512 |
| Conv2d + Leaky ReLU | 1 × 3, 1, 512 | 32 × 4 × 512 |
| Max Pooling | 2 × 2 | 16 × 2 × 512 |
| Conv2d + Leaky ReLU | 1 × 3, 1, 512 | 16 × 2 × 512 |
| Conv2d + Leaky ReLU | 1 × 3, 1, 512 | 16 × 2 × 512 |
| Max Pooling | 2 × 2 | 8 × 1 × 512 |
| FC: L-ReLU + | — | 4096 |
| Dropout | | |
| FC: L-ReLU + | — | 4096 |
| Dropout | | |
| FC: L-ReLU + | — | 1000 |
| Dropout | | |
| Softmax | — | N classes |

Another aspect of the present disclosure provides a method of determining a cancer condition of a test subject of a species, the method comprising at a computer system comprising at least one processor and a memory storing at least one program for execution by the at least one processor. The at least one program can comprise instructions for obtaining a dataset, in electronic form, where the dataset can comprise a corresponding methylation pattern of each respective fragment in a plurality of fragments. The corresponding methylation pattern of each respective fragment (i) can be determined by a methylation sequencing of one or more nucleic acid samples of the respective fragment in a biological sample obtained from the test subject and (ii) can comprise a methylation state of each CpG site in a corresponding plurality of CpG sites in the respective fragment.

The at least one program can further comprise instructions for obtaining a plurality of patches, where each respective patch in the plurality of patches can comprise a first channel and represents a corresponding independent set of CpG sites in a reference genome of the species. Each respective CpG site in the corresponding independent set of CpG sites can correspond to a predetermined location in the reference genome. The first channel for a respective patch can comprise a plurality of instances of a first plurality of parameters, where each instance of the first plurality of parameters includes a parameter for a methylation status of a respective CpG site in the corresponding independent set of CpG sites for the respective patch. The at least one program can further comprise instructions for assigning all or a portion of each respective fragment in the plurality of fragments to a respective patch in the plurality of patches based upon a match between CpG sites of the respective fragment and the corresponding independent set of CpG sites of the single respective patch. The at least one program can further comprise instructions for applying each respective patch in the plurality of patches to a corresponding trained model in a plurality of models thereby determining the cancer condition in the test subject.

Respective fragment in the plurality of fragments can be a unique molecular fragment that aligns to different genomic location(s) or can include a different methylation pattern. Specifically, a fragment can be a unique molecular fragment that aligns to a genomic location, such that the assigning of all or a portion of each respective fragment to a respective patch can be based upon a match between CpG sites of the respective fragment and the corresponding independent set of CpG sites of the respective patch, rather than based upon a methylation pattern of the respective fragment.

The method can use a plurality of patches. The at least one program may not comprise instructions for constructing the patch by populating, for each respective fragment that aligns to the first independent set of CpG sites, an instance of all or a portion of the first plurality of parameters based on the methylation pattern of the respective fragment. In contrast, the obtained plurality of patches can be previously constructed.

Assigning all or a portion of each respective fragment in the plurality of fragments to a respective patch in the plurality of patches based upon a match between CpG sites of the respective fragment and the corresponding independent set of CpG sites of the respective patch can comprise, for a respective fragment in the plurality of fragments assigned to the single respective patch: i) identifying, within an instance of the first plurality of parameters of the first channel of the single respective patch, parameters, corresponding to the CpG sites in the respective fragment, that have not previously been assigned methylation states by another fragment in the plurality of fragments; and ii) assigning for each parameter, among the identified parameters, in the instance of the first plurality of parameters of the first channel of the single respective patch, that aligns to a respective CpG site of the respective fragment, the methylation state of the respective CpG site of the respective fragment.

The nucleic acid samples can include cell-free nucleic acid samples. The biological sample can be processed to extract cell-free nucleic acids in preparation for sequencing analysis. Details of the biological sample are described elsewhere herein. For example, cell-free nucleic acid can be extracted from a blood sample collected from a subject in K2 EDTA tubes. Samples can be processed within two hours of collection by double spinning of the blood first at ten minutes at 1000 g then plasma ten minutes at 2000 g. The plasma can then be stored in 1 ml aliquots at −80° C. In this way, a suitable amount of plasma (e.g., 1-5 ml) can be prepared from the biological sample for the purposes of cell-free nucleic acid extraction. Cell-free nucleic acid can be extracted using the QIAamp Circulating Nucleic Acid kit (Qiagen) and eluted into DNA Suspension Buffer (Sigma). The purified cell-free nucleic acid can be stored at −20° C. until use. One or more methods can be used to prepare cell-free nucleic acid using biological methods for the purpose of sequencing.

The time between obtaining a biological sample and performing an assay, such as a sequence assay, can be optimized to improve the sensitivity and/or specificity of the assay or method. A biological sample can be obtained immediately before performing an assay. A biological sample can be obtained, and stored for a period of time (e.g., hours, days or weeks) before performing an assay. An assay can be performed on a sample within 1 day, 2 days, 3 days, 4 days, 5 days, 6 days, 1 week, 2 weeks, 3 weeks, 4 weeks, 5 weeks, 6 weeks, 7 weeks, 8 weeks, 3 months, 4 months, 5 months, 6 months, 1 year, or more than 1 year after obtaining the sample from the training subject.

The nucleic acids for each respective subject can be obtained by targeted panel sequencing in which the sequence reads taken from a biological sample of a subject in order to form a dataset comprising at least 50,000× sequencing depth for this targeted panel of genes, at least 55,000× sequencing depth for this targeted panel of genes, at least 60,000× sequencing depth for this targeted panel of genes, or at least 70,000× sequencing depth for this targeted panel of genes. The targeted panel of genes can be between 450 and 500 genes. In some embodiments, the targeted panel of genes is within the range of 500±5 genes, within the range of 500±10 genes, or within the range 500±25 genes.

The sequencing method can comprise whole genome bisulfite sequencing. The whole genome bisulfite sequencing can identify one or more methylation state vectors as described, for example, U.S. patent application Ser. No. 16/352,602, entitled “Anomalous Fragment Detection and Classification,” filed Mar. 13, 2019, or in accordance with any of the techniques disclosed in U.S. Provisional Patent Application No. 62/847,223, entitled “Model-Based Featurization and Classification,” filed May 13, 2019, each of which is hereby incorporated by reference. The plurality of nucleic acids can be generated from a CCGA 1 dataset, as described in Example 1 below. The plurality of nucleic acids can be processed to obtain copy number values that are used to train a classifier (e.g., patch CNN classifier). A test dataset obtained from a biological sample from a subject can then be inputted into the trained classifier to determine whether the subject has a disease condition, and, in some embodiments, a type, stage and/or other characteristics of the disease condition. Genomic regions with high variability or low mappability can be excluded.

The targeted sequencing can include targeted DNA methylation sequencing. The targeted DNA methylation sequencing can be performed in various ways. Different enzymatic treatments and combination with chemical treatment(s) can convert either methylated cytosines or unmethylated cytosines. For example, the targeted DNA methylation sequencing can detect one or more 5-methylcytosine (5mC) and/or 5-hydroxymethylcytosine (5hmC) in the plurality of nucleic acids (block 410). As another example, the targeted DNA methylation sequencing may comprise conversion of one or more unmethylated cytosines or one or more methylated cytosines, in the plurality of nucleic acids, to a corresponding one or more uracils. As another example, the targeted DNA methylation sequencing may comprise conversion of one or more unmethylated cytosines, in the plurality of nucleic acids, to a corresponding one or more uracils, and the DNA methylation sequence reads out the one or more uracils as one or more corresponding thymines. The targeted DNA methylation sequencing can comprise conversion of one or more methylated cytosines, in the plurality of nucleic acids, to a corresponding one or more uracils, and the DNA methylation sequence reads out the one or more 5mC or 5hmC as one or more corresponding thymines.

FIG. 8B depicts another exemplary flowchart describing a method 850 of determining a cancer condition of a test subject. The method can be performed by the environment 500 and/or the processing system 560 disclosed herein.

Step 852 of the method 850 can include obtaining, via one or more processors, a training dataset from one or more training subjects. The training dataset can comprise one or more training methylation patterns associated with a plurality of fragments in one or more biological samples obtained from the one or more training subjects and one or more predetermined cancer conditions associated with the one or more training methylation patterns. The training dataset can include any biological or genomic information of the training subjects, including, but not limited to, information relating to the primary nucleic acid sequence of all or a portion of the genome (e.g., the presence or absence of a nucleotide polymorphism, indel, sequence rearrangement, mutational frequency, etc.), the copy number of one or more particular nucleotide sequences within the genome (e.g., copy number, allele frequency fractions, single chromosome or entire genome ploidy, etc.), the epigenetic status of all or a portion of the genome (e.g., covalent nucleic acid modifications such as methylation, histone modifications, nucleosome positioning, etc.), and the expression profile of the organism's genome (e.g., gene expression levels, isotype expression levels, gene expression ratios, etc.).

The one or more training methylation patterns can be determined by at least one methylation sequencing of one or more nucleic acid samples comprising the plurality of fragments in the one or more biological samples obtained from the one or more training subjects. The one or more training methylation patterns can comprise at least one methylation state of each CpG site in the plurality of fragments in the one or more biological samples obtained from the one or more training subjects. The training methylation patterns can be the methylation patterns of the training subjects. The training subject can be any subject whose information is used to train a computational model. The training subject can be different from the test subject. Details of the subject, the computational model, the methylation pattern, and how to determine the methylation pattern are described elsewhere herein. The one or more predetermined cancer conditions can be any cancer conditions described elsewhere herein.

Step 854 of the method 850 can comprise constructing, via the one or more processors, one or more patches based on the training dataset. Each patch of the one or more patches can comprise one or more channels. Each patch of the one or more patches can represent one or more CpG sites in a reference genome of the species. Each CpG site of the CpG sites can correspond to a predetermined location in the reference genome. Each patch or a first patch of the one or more patches can represent a first independent set of CpG sites in a reference genome of the species. Each respective CpG site in the first independent set of CpG sites can correspond to a predetermined location in the reference genome. The constructing can comprise populating or filling, for each respective fragment in the plurality of fragments in one or more biological samples obtained from the one or more training subjects that aligns to the first independent set of CpG sites, an instance of all or a portion of the first plurality of parameters based on the training methylation pattern of the respective fragment. Details of the first independent set of CpG sites, the instance, the parameters, the one or more patches, and how to construct the one or more patches are further described elsewhere herein.

The one or more channels can comprise a first channel. The first channel can comprise a plurality of instances of a first plurality of parameters. Each instance of the first plurality of parameters can include a parameter for a methylation status of a respective CpG site in a first independent set of CpG sites for a patch of the one or more patches. In this situation, the constructing, for a respective fragment in the plurality of fragments in one or more biological samples obtained from the one or more training subjects, can comprise: i) identifying, within an instance of the first plurality of parameters of the first channel, parameters, corresponding to the CpG sites in the respective fragment, that have not previously been assigned methylation states based on another fragment in the plurality of fragments; and ii) assigning for each parameter, among the identified parameters, that aligns to a corresponding CpG site of the respective fragment, the methylation state of the corresponding CpG site of the respective fragment. Further details of how to identify parameters and how to assign the methylation state are described elsewhere herein.

The one or more channels can comprise a second channel. The second channel can comprise information different from the first channel. The second channel can comprise a corresponding instance of a second plurality of parameters for each instance of the first plurality of parameters. Each instance of the second plurality of parameters can include a parameter for a first characteristic, other than CpG methylation state, of a respective CpG site in the first independent set of CpG sites for the first patch. The one or more channels can further comprise a third channel. The third channel can comprise information different from the first/second channel. The third channel can comprise a corresponding instance of a third plurality of parameters for each instance of the first plurality of parameters. Each instance of the third plurality of parameters can include a parameter for a second characteristic of a respective CpG site in the first independent set of CpG sites. The number of the one or more channels can be at least 1, 2, 3, 4, 5, 6, 7, 8, 9, 10 or more. In some embodiments, the number of one or more channels can be at most 10, 9, 8, 7, 6, 5 or less. In case that the number of the one or more channels is larger than 1, each channel of the one or more channels can include unique information associated with one type of characteristics (e.g., a first characteristics). For instance, each of the 6 channels in FIG. 6B can include information associated with methylation state, beta controls, beta sample, p-value, multiplicity, or priors. In this example, each channel of the 6 channels can include information different from other channels. Details of the one or more channels and the characteristics (e.g., first characteristics, second characteristics) are described elsewhere herein.

Prior to step 854, or at any stage of determining a cancer condition, the method 850 can comprise pruning the plurality of fragments in one or more biological samples obtained from the one or more training subjects by removing from the plurality of fragments each respective fragment, whose corresponding methylation pattern across a corresponding plurality of CpG sites in the respective fragment, has a p-value that fails to satisfy a p-value threshold. Details of the p-value, the p-value threshold, and pruning the plurality of fragments are described elsewhere herein.

Step 856 of the method 850 can comprise training, via the one or more processors, a computational model based on the one or more patches and the training dataset. The computational model can comprise a first stage model and a second stage model. The first stage model can comprise one or more convolutional neural networks (CNNs). The convolutional neural networks can include a pre-trained convolutional neural network. The pre-trained CNN can use one or more layers of a convolutional neural net that has been trained on pixelated image data (e.g., RGB pixelated images). Examples of such pre-trained CNN model can include, but are not limited to, LeNet, AlexNet, VGG-11, VGGNet 16, GoogLeNet, or ResNet. The pre-trained convolutional neural network can comprise a customized pre-trained CNN. The customized pre-trained CNN can include a customized VGG-11 convolutional neural network. The customized VGG-11 convolutional neural network can comprise customized filter size and activation function. Details of the first stage model, the CNNs, the second stage model, the pre-trained CNN, and the customized VGG-11 are further described elsewhere herein.

Step 858 of the method 850 can comprise obtaining, via the one or more processors, a test dataset from the test subject. The test dataset can comprise one or more testing methylation patterns of a plurality of fragments in the one or more biological samples obtained from the test subject. The testing dataset can include any biological or genomic information of the testing subjects. Details of such biological and genomic information are described elsewhere herein. The one or more testing methylation patterns can be determined by a methylation sequencing of one or more nucleic acid samples comprising the plurality of fragments in a biological sample obtained from the test subject. The one or more testing methylation pattern can comprise at least one methylation state of each CpG site in the plurality of fragments in the biological sample obtained from the test subject. The testing methylation patterns can be the methylation patterns of the testing subject.

Step 860 of the method 850 can comprise determining, via the one or more processors, the cancer condition of the test subject based on the test dataset and the computational model. The determining can comprise applying at least the first patch to a classifier thereby determining the cancer condition in the test subject. The computational model can predict cancer versus non-cancer and/or tissue-of-origin based on the test dataset. The computational model can perform a multi-class prediction that discriminates between cancer/non-cancer/uninformative, tissue-of-origin, organ-of-origin, cancer type, and/or cancer stage.

Any methods described herein can further comprise updating the computational model/classifier using one or more biological priors. The biological priors can include, but are not limited to, geographic information, smoker/non-smoker, disease condition stage, age group, detectability of a disease condition, and/or gender (biological sex). The updated computational model can include a classifier (e.g., a multi-class classifier) and a mathematical calculation (e.g., matrix computations) for application in general population. In this situation, the mathematical calculation can be applied before or after the classifier. In some embodiments, the updated computational model can be a classifier including a mathematical calculation for application in general population. In this situation, the mathematical calculation can be incorporated into the classifier and trained with the classifier. The classifier can include any machine learning or statistical models disclosed elsewhere herein that can perform classification based on any data or information disclosed herein. In case that the classifier includes one or more patches for a convolutional neural network, information associated with the one or more biological priors may or may not be incorporated into one or more channels of the one or more patches. The mathematical calculation can include a Naïve Bayesian statistical calculation, where the one or more biological priors can be used to calculate posterior probabilities. The mathematical calculation can be a mechanism to modify the computational model, as described elsewhere herein, for application in different target populations (e.g., patients in different continents). The updated computational model can include information representing the frequency of cancer and relative frequency of cancer types in different target populations. The frequency of cancer can include a frequency distribution of training dataset. The updated computational model can enable generalizable performance across heterogenous studies (e.g., STRIKE as described elsewhere herein).

In some embodiments, to update the computational model, one or more biological priors can include disease condition stage (e.g., cancer stage), detectability of a disease condition (e.g., detectability of cancer), and/or gender (biological sex). In this situation, the mathematical calculation can combine i) gender-specific incidence and stage-specific incidence of cancer in the general population and ii) the detectability of cancer across different stages (e.g., from tumor fraction results in CCGA1). The mathematical calculation can include multiplying, adding, dividing, and/or subtracting between i) the gender-specific incidence and stage-specific incidence of cancer in the general population and ii) the detectability of cancer across different stages. In some embodiments, the gender-specific incidence and the stage-specific incidence of cancer can be scaled based on the detectability of cancer across different stages. The gender-specific incidence can include any information (e.g., a probability) associated with gender/biological sex of the training or test subject. The gender-specific incidence can be used because some types of cancers (e.g., breast cancer) are gender-specific. The stage-specific incidence of cancer can include any information (e.g., a probability) associated with a cancer stage of the training or test subject. The detectability of cancer can be determined based on tumor fraction. For instance, if certain type of cancer is low shedding (e.g., the tumor fraction of the type of cancer is low in the blood sample), the value of the detectability of cancer can be low.

If the updated computational model includes a classifier and a mathematical calculation, the classifier can be trained with training dataset and the mathematical calculation may not be trained with the training dataset. If the updated computational model is a classifier including a mathematical calculation, the classifier and the mathematical calculation can be trained with the training dataset. In this situation, the one or more biological priors can be constructed as a one-dimensional or multi-dimensional matrix that is able to be combined with training dataset to input into the classifier.

The method can further comprise transmitting, via the one or more processors, the disease condition (e.g., cancer condition) to an electronic record associated with a user device of the test subject. The disease condition can be passed, forwarded, or transmitted using any suitable methods including memory sharing, message passing, token passing, or network transmission. The disease condition can be transmitted via text display, photographic display, hyperlink, video/audio displays, SMS, messaging application or service, email, or any other suitable mechanism to a test subject, health professionals, or other party. The disease condition can be shown on a graphical user interface (e.g., a graphical user interface 550). The graphical user interface can be configured to provide a user (e.g., health professionals) with graphic showings of, for example, the disease conditions and treatment suggestion or recommendation of preventive steps based on the disease conditions. The graphical user interface can enable user interactions with particular tasks (e.g., reviewing the disease conditions and adjusting treatment plans). The disease condition (e.g., the cancer condition) can comprise level of cancer, tissues of origin, and metastatic disease status. Details of the level of cancer and tissues of origin are described elsewhere herein.

Metastasis disease status can represent a metastasis process of spreading cancer cells to new areas of the body through the lymph system, bloodstream, or other route. In addition to tissues of origin (TOO), the cancer condition can provide additional information of the metastatic disease status associated with cancer spreading from the TOO. Such metastatic disease status can be either indicative of TOO or indicative of the spread of cancer cells to other organs in the body (e.g., tumor-adjacent tissues). CfDNA fragments can originate from cell death, and the presence of the cfDNA fragments can indicate tissue injury and cell death in other regions (e.g., tumor-adjacent tissues or other organs in the body affected by an invading metastatic disease) other than the TOO.

The detection of cancer and cfDNA fragments from cells affected by a metastasis process can be implemented by using the classifier or the computational model described elsewhere herein. Clinical knowledge can be implemented in a multi-step analysis to distinguish between cfDNA fragments from TOO and those from adjacent tissues at a metastatic site. Clinical knowledge can capture how frequent cancers of a known tissue of origin metastasize to other organs or tissues. Such information can be obtained from cancer registries. For example, SEER Research Data 1975-2017 collects the presence of a distant metastasis to bone, brain, liver. lung, lymph nodes or other sites at time of diagnosis. See, also, Budczies et al., 2014, “The landscape of metastatic progression patterns across major human cancers,” Oncotarget, 2014 Nov. 4; 6(1):570-83, which is hereby incorporated by reference. To determine the metastasis disease status, any methods described herein can further comprise two steps to separately identify TOO and metastatic process using fragment-level sequencing data. A first step can include any methods (e.g., method 800 or method 850) described herein to determine TOO of a test subject via a classifier/computational model using a plurality of fragments (e.g., cfDNA fragments) in one or more biological samples obtained from the test subject. A second step can include analyzing the plurality of fragments via the classifier/computational model in the first step to detect metastasis disease status of other tissues distant to the tissues of origin that are more likely affected by a metastatic process associated with the determined TOO. The other tissues can be determined based on clinical knowledge.

For example, if the first step determines the tissue of origin of a test subject is breast (or the test subject has breast cancer) via a classifier using a plurality of fragments in one or more biological samples obtained from the test subject, then the second step can include analyzing the plurality of fragments with the classifier to detect the presence of non-cancerous cells affected by a metastasis process to other tissues, such as liver, brain, bone, or lung, which are clinically-known common organs affected by breast cancer metastasis. Similarly, in one example, if the first step determines the tissue of origin of a test subject is lung (or the test subject has lung cancer) via a classifier using a plurality of fragments in one or more biological samples obtained from the test subject, the second step can include analyzing the plurality of fragments with the classifier to detect the presence of non-cancerous cells affected by a metastasis process to other tissues, such as liver, bones, brain, or adrenal glands, which are clinically-known common organs affected by lung cancer metastasis. In another example, if the first step determines the tissue of origin of a test subject is colon or rectum (or the test subject has colorectal cancer) via a classifier using a plurality of fragments in one or more biological samples obtained from the test subject, the second step can include analyzing the plurality of fragments with the classifier to detect the presence of non-cancerous cells affected by a metastasis process to other tissues, such as liver, lung, brain, and peritoneum, which are clinically-known common organs affected by colorectal cancer metastasis. In a further example, if the first step determines the tissue of origin of a test subject is prostate (or the test subject has prostate cancer) via a classifier using a plurality of fragments in one or more biological samples obtained from the test subject, the second step can include analyzing the plurality of fragments with the classifier to detect the presence of non-cancerous cells affected by a metastasis process to other tissues, such as spread to bone, liver, and lung, which are clinically-known common organs affected by prostate cancer metastasis.

The classifier used in the first step can be the same as the classifier used in the second step. For instance, the classifier can provide normalized probabilities of cancer (e.g., a value between 0 and 1) for a plurality of tissues. Based on the normalized probabilities. a rank of the plurality of tissues can be created. In this situation, the tissue ranked the highest can be the tissue of origin, and the tissue ranked the second-highest with a normalized probability larger than 0 (e.g., >0.1) can be other tissue distant to the tissue of origin that is more likely affected by a metastatic process. Example 10 provides further details. While the classifier is trained on cfDNA samples from tumor cells, the methylation signal of tumor-adjacent normal tissue can sometimes be similar enough to result in visible scores.

In some embodiments, the classifier used in the second step can be different from the classifier used in the first step. In this situation, the classifier used in the second step can be a disease-specific classifier. A training dataset collected from non-cancerous cells and/or patients with known cancer and site of metastasis can be used to train the disease-specific classifier for metastatic sites. The combination of a classifier for determining TOO in the first step and a disease-specific classifier in the second step can provide higher accuracy and increased robustness compared to using a classifier for both the first and second steps.

The methods, systems, computational model, and/or classifier of the present disclosure can be used to detect the presence (or absence) of cancer, tissue of origin, monitor cancer progression or recurrence, monitor therapeutic response or effectiveness, determine a presence or monitor minimum residual disease (MRD), or any combination thereof. In one example a computational model and/or classifier can be used to generate a likelihood or probability score (e.g., from 0 to 1) that a feature vector is from a subject with cancer. The likelihood or probability score can be one type of disease condition. The probability score can be compared to a threshold probability to determine whether or not the subject has cancer. In other embodiments, the likelihood or probability score can be assessed at different time points (e.g., before or after treatment) to monitor disease progression or to monitor treatment effectiveness (e.g., therapeutic efficacy). In still other embodiments, the likelihood or probability score can be used to make or influence a clinical decision (e.g., diagnosis of cancer, treatment selection, assessment of treatment effectiveness, etc.). For example, if the likelihood or probability score exceeds a threshold, a health professional can prescribe an appropriate treatment.

If the likelihood or probability score is assessed at different time points, the first time point can be before a cancer treatment (e.g., before a resection surgery or a therapeutic intervention), and the second time point can be after a cancer treatment (e.g., after a resection surgery or therapeutic intervention). In this situation, the method can further comprise monitoring the effectiveness of the treatment. For example, if the second likelihood or probability score decreases compared to the first likelihood or probability score, then the treatment can be considered to have been successful. However, if the second likelihood or probability score increases compared to the first likelihood or probability score, then the treatment can be considered to have not been successful. In other embodiments, both the first and second time points can be before a cancer treatment (e.g., before a resection surgery or a therapeutic intervention). In still other embodiments, both the first and the second time points can after a cancer treatment (e.g., before a resection surgery or a therapeutic intervention) and the method can further comprise monitoring the effectiveness of the treatment or loss of effectiveness of the treatment. In still other embodiments, cfDNA samples may be obtained from a cancer patient at a first and second time point and analyzed. e.g., to monitor cancer progression, to determine if a cancer is in remission (e.g., after treatment), to monitor or detect residual disease or recurrence of disease, or to monitor treatment (e.g., therapeutic) efficacy.

Test samples can be obtained from a cancer patient over any set of time points and analyzed in accordance with the methods of the disclosure to monitor a cancer state in the patient. The first and second time points can be separated by an amount of time that ranges from about 15 minutes up to about 30 years, such as about 30 minutes, such as about 1, 2, 3, 4, 5, 6, 7, 8, 9, 10, 11, 12, 13, 14, 15, 16, 17, 18, 19, 20, 21, 22, 23, or about 24 hours, such as about 1, 2, 3, 4, 5, 10, 15, 20, 25 or about 30 days, or such as about 1, 2, 3, 4, 5, 6, 7, 8, 9, 10, 11, or 12 months, or such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or about 30 years. In other embodiments, test samples can be obtained from the patient at least once every 3 months, at least once every 6 months, at least once a year, at least once every 2 years, at least once every 3 years, at least once every 4 years, or at least once every 5 years.

Information obtained from any method described herein (e.g., the likelihood or probability score, a disease condition) can be used to make or influence a clinical decision (e.g., diagnosis of cancer, treatment selection, assessment of treatment effectiveness, etc.). For example, if the likelihood or probability score exceeds a threshold, a health professional can prescribe an appropriate treatment (e.g., a resection surgery, radiation therapy, chemotherapy, and/or immunotherapy) via a graphical user interface on health professional's user device (e.g., user device 520) or any other communication medium (e.g., a phone call or a mail). Information such as a likelihood or probability score can be provided as a readout to a physician or subject via the graphical user interface. In one example, if the likelihood or probability score is greater than or equal to 0.6, one or more appropriate treatments can be prescribed. In another embodiments, if the likelihood or probability score is greater than or equal to 0.65, greater than or equal to 0.7, greater than or equal to 0.75, greater than or equal to 0.8, greater than or equal to 0.85, greater than or equal to 0.9, or greater than or equal to 0.95, one or more appropriate treatments can be prescribed.

The treatment can include one or more cancer therapeutic agents including a chemotherapy agent, a targeted cancer therapy agent, a differentiating therapy agent, a hormone therapy agent, and an immunotherapy agent. For example, the treatment can be one or more chemotherapy agents including alkylating agents, antimetabolites, anthracyclines, anti-tumor antibiotics, cytoskeletal disruptors (taxans), topoisomerase inhibitors, mitotic inhibitors, corticosteroids, kinase inhibitors, nucleotide analogs, platinum-based agents and any combination thereof. The treatment can include one or more targeted cancer therapy agents including signal transduction inhibitors (e.g., tyrosine kinase and growth factor receptor inhibitors), histone deacetylase (HDAC) inhibitors, retinoic receptor agonists, proteosome inhibitors, angiogenesis inhibitors, and monoclonal antibody conjugates. The treatment can include one or more differentiating therapy agents including retinoids, such as tretinoin, alitretinoin and bexarotene. The treatment can include one or more hormone therapy agents including anti-estrogens, aromatase inhibitors, progestins, estrogens, anti-androgens, and GnRH agonists or analogs. The treatment can include one or more immunotherapy agents including monoclonal antibody therapies such as rituximab (RITUXAN) and alemtuzumab (CAMPATH), non-specific immunotherapies and adjuvants, such as BCG, interleukin-2 (IL-2), and interferon-alfa, immunomodulating drugs, for instance, thalidomide and lenalidomide (REVLIMID). An appropriate cancer therapeutic agent cab be selected based on characteristics such as the type of tumor, cancer stage, previous exposure to cancer treatment or therapeutic agent, and other characteristics of the cancer.

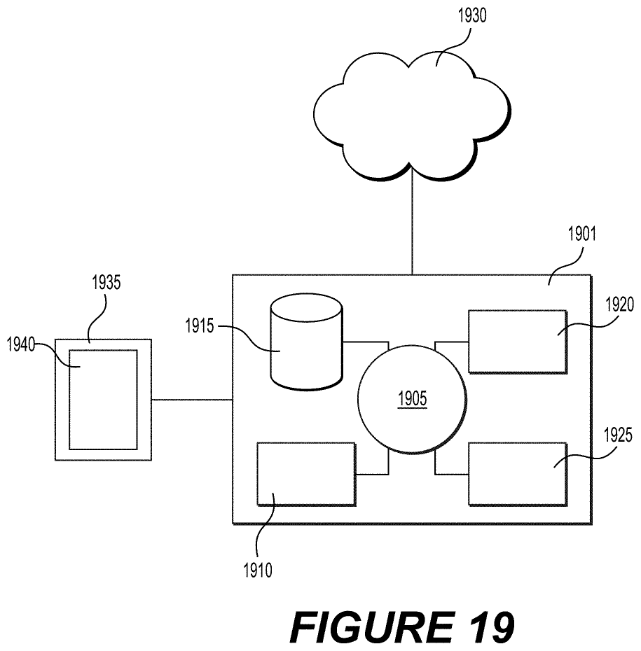

FIG. 19 shows an exemplary computer system 1901 that is programmed or otherwise configured to determine a disease condition of a test subject of a species. The computer system 1901 can implement and/or regulate various aspects of the methods provided in the present disclosure, such as, for example, performing the method of determining a cancer condition of a test subject as described herein, performing various steps of the bioinformatics analyses of training dataset and testing dataset as described herein, integrating data collection, analysis and result reporting, and data management. The computer system 1901 can be an electronic device of a user or a computer system that is remotely located with respect to the electronic device. The electronic device can be a mobile electronic device.

The computer system 1901 can include a central processing unit (CPU, also “processor” and “computer processor” herein) 1905, which can be a single core or multi core processor, or a plurality of processors for parallel processing. The computer system 1901 can also include memory or memory location 1910 (e.g., random-access memory, read-only memory, flash memory), electronic storage unit 1915 (e.g., hard disk), communication interface 1920 (e.g., network adapter) for communicating with one or more other systems, and peripheral devices 1925, such as cache, other memory, data storage and/or electronic display adapters. The memory 1910, storage unit 1915, interface 1920 and peripheral devices 1925 can be in communication with the CPU 1905 through a communication bus (solid lines), such as a motherboard. The storage unit 1915 can be a data storage unit (or data repository) for storing data. The computer system 1901 can be operatively coupled to a computer network (“network”) 1930 with the aid of the communication interface 1920. The network 1930 can be the Internet, an internet and/or extranet, or an intranet and/or extranet that is in communication with the Internet. The network 1930 in some cases can be a telecommunication and/or data network. The network 1930 can include one or more computer servers, which can enable distributed computing, such as cloud computing. The network 1930, in some cases with the aid of the computer system 1901, can implement a peer-to-peer network, which may enable devices coupled to the computer system 1901 to behave as a client or a server.

The CPU 1905 can execute a sequence of machine-readable instructions, which can be embodied in a program or software. The instructions may be stored in a memory location, such as the memory 1910. The instructions can be directed to the CPU 1905, which can subsequently program or otherwise configure the CPU 1905 to implement methods of the present disclosure. Examples of operations performed by the CPU 1905 can include fetch, decode, execute, and writeback.

The CPU 1905 can be part of a circuit, such as an integrated circuit. One or more other components of the system 1901 can be included in the circuit. In some cases, the circuit is an application specific integrated circuit (ASIC).

The storage unit 1915 can store files, such as drivers, libraries and saved programs. The storage unit 1915 can store user data, e.g., user preferences and user programs. The computer system 1901 in some cases can include one or more additional data storage units that are external to the computer system 1901, such as located on a remote server that is in communication with the computer system 1901 through an intranet or the Internet.

The computer system 1901 can communicate with one or more remote computer systems through the network 1930. For instance, the computer system 1901 can communicate with a remote computer system of a user (e.g., a Smart phone installed with application that receives and displays results of sample analysis sent from the computer system 1901). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1901 via the network 1930.

Methods as described herein can be implemented by way of machine (e.g., computer processor) executable code stored on an electronic storage location of the computer system 1901, such as, for example, on the memory 1910 or electronic storage unit 1915. The machine executable or machine readable code can be provided in the form of software. During use, the code can be executed by the processor 1905. In some cases, the code can be retrieved from the storage unit 1915 and stored on the memory 1910 for ready access by the processor 805. In some situations, the electronic storage unit 1915 can be precluded, and machine-executable instructions are stored on memory 1910.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that include a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1901 can include or be in communication with an electronic display 1935 that includes a user interface (UI) 1940 for providing, for example, results of sample analysis, such as, but not limited to graphic showings of the stage of processing the input sequencing data, output sequencing data, and further classification of pathology (e.g., type of disease or cancer and level of cancer). Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1905. The algorithm can perform any step of the methods described here.

### Example 1—Circulating Cell-Free Genome Atlas Study (CCGA)

The Circulating Cell-Free Genome Atlas Study (CCGA; NCT02889978) is a prospective, multi-center, observational cfDNA-based early cancer detection study that has enrolled 15,254 demographically-balanced participants at 141 sites. Blood samples were collected from the 15,254 enrolled participants (56% cancer, 44% non-cancer).

In a first cohort (pre-specified substudy) (CCGA1), plasma cfDNA extraction were obtained from 3,583 CCGA and STRIVE participants (CCGA: 1,530, 884 non-cancer; STRIVE 1,169 non-cancer participants). STRIVE is a multi-center, prospective, cohort study enrolling women undergoing screening mammography (99,259 participants enrolled). Three sequencing assays were performed on the blood drawn from each participant: paired cfDNA and white blood cell (WBC) targeted sequencing (507 genes, 60,000×) for single nucleotide variants/indels (the ART sequencing assay), paired cfDNA and WBC whole-genome sequencing (WGS, 30×) for copy number variation, and cfDNA whole-genome bisulfite sequencing (WGBS, 30×) for methylation.

In a second pre-specified substudy (CCGA-2), a targeted, rather than whole-genome, bisulfite sequencing assay was used to develop a classifier of cancer versus non-cancer and tissue-of-origin based on a targeted methylation sequencing approach. For CCGA2, 3,133 training participants and 1,354 validation samples (775 having cancer; 579 not having cancer as determined at enrollment, prior to confirmation of cancer versus non-cancer status) were used. Plasma cfDNA was subjected to a bisulfite sequencing assay targeting the most informative regions of the methylome, as identified from a unique methylation database and prior prototype whole-genome and targeted sequencing assays, to identify cancer and tissue-defining methylation signal. Of the original 3,133 samples reserved for training, 1,308 samples were deemed clinically evaluable and analyzable. Analysis was performed on a primary analysis population n=927 (654 cancer and 273 non-cancer) and a secondary analysis population n=1,027 (659 cancer and 373 non-cancer).

Classification of validation samples was performed using the methylation states of nucleic acid fragments. For binary classification, observed nucleic acid fragments were assigned a relative probability of originating from cancer. Similarly, for tissue-of-origin classification, observed nucleic acid fragments were assigned a relative probability of originating from a particular tissue. Nucleic acid fragments characteristic of cancer and tissue-of-origin were combined across targeted regions to classify cancer versus non-cancer and identify tissue-of-origin. For binary cancer classification, clinical sensitivity was estimated at 99% specificity. For tissue-of-origin, two independent models, one with and one without the methylation database, were fitted; reported tissue-of-origin results reflect percent agreement between predicted and true tissue-of-origin among cases classified as cancer at 99% specificity.

### Example 2: Classifier Training and Performance

A training dataset was generated from 2079 samples. The patch-CNN classifier that was used included 543 patches. Thus, 543 patches per sample were calculated for a total of approximately 1 million Tensorflow (Google) training samples. This dataset was used to train a classifier for Patch-CNN. The 2079 samples used in the training dataset comprised multiple studies, including CCGA1 (1529 samples), CCGA2 (328 samples) and Conversant (221 samples), as well as multiple biospecimens, including cell-free DNA (cfDNA) (1343 samples), formalin-fixed paraffin-embedded (FFPE) (561 samples), disseminated tumor cells (DTC) (87 samples), and cryopreserved (59 samples).

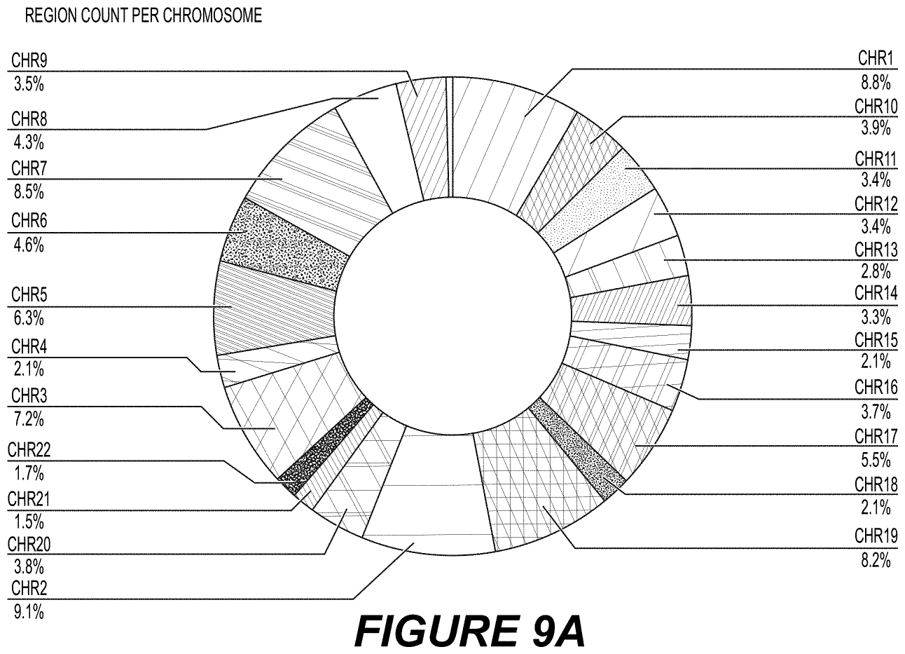

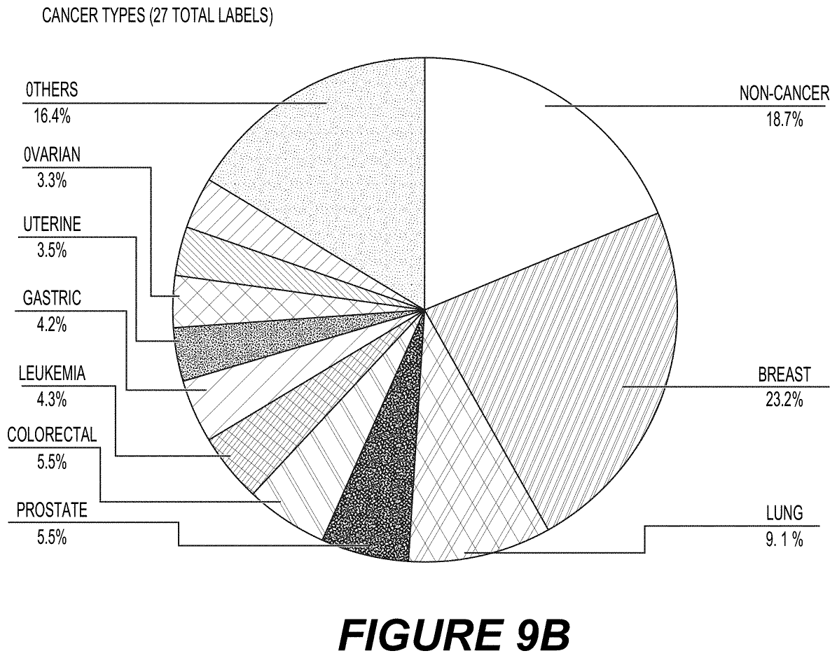

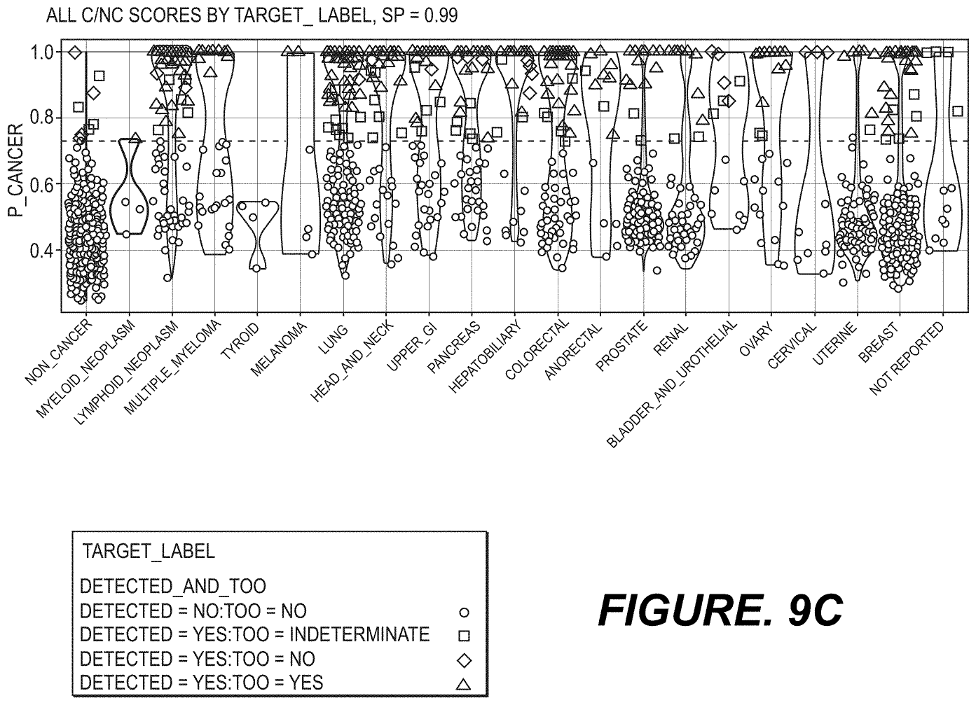

Patch selection was performed using a mutual information method, comprising a selection of the top 5 high-mutual-information genomic regions for every cancer type pair. Mutual information describes the relationship between two classification types such that, for example, a high-mutual-information region for a pair of cancer types comprises CpG sites that are highly discriminative between samples of the first cancer type and samples of the second cancer type. Region representation per chromosome used for patch selection in some embodiments is illustrated in FIG. 9A. For each selected region, neighboring CpG sites were merged and the regions were padded by 100 sites, keeping the CpGs of interest centered. Regions were then selected such that all CpG sites were covered, with the exception of regions with no control group coverage using young healthy samples from CCGA 1. In some instances where multiple pairwise comparisons were possible (e.g., for a multiclass classifier), high-mutual-information regions were selected such that highly discriminative sites for all possible cancer type pairs were represented in the model.

Training was performed using 8-fold cross-validation stratified by cancer type and stage (e.g., by binning all samples into 8 bins of equal size such that there is an even distribution across all bins of cancer samples, non-cancer samples, cancer stages I-IV, and/or tissue-of-origin, among others). During cross-validation, the model was trained on seven bins and evaluated on the eighth bin, with validation repeated 8 times such that each of the 8 bins was evaluated separately. Cancer types used for stratification in some embodiments are illustrated, for example, in FIG. 9B, including ovarian, uterine, gastric, leukemia, colorectal, prostate, breast, lung, other cancer types and non-cancer types.

The performance of a classifier to detect cancer versus non-cancer (“DETECT”) and tissue-of-origin (“TOO”) was assessed for a panel of cancer types as illustrated in FIG. 9C for the case of TOO. For additional details, see Oxnard et al., “Simultaneous Multi-cancer Detection and Tissue of Origin (TOO) Localization Using Targeted Bisulfite Sequencing of Plasma Cell-free DNA (cfDNA),” American Society of Clinical Oncology (ASCO) Breakthrough, Oct. 11-13, 2019, Bangkok, Thailand, which is hereby incorporated by reference. True positives are denoted by triangles and true negatives are denoted by circles, with false positives and indeterminate samples denoted by diamonds and squares, respectively. Samples were labeled cancer or non-cancer, and cancer samples were further labeled with cancer type. All samples were detected with 99% specificity. FIG. 9C illustrates the presence of false positives (diamonds) in cancer samples that were likely due to the presence of undiagnosed blood cancers. The results suggest that further optimization of the model can be used to avoid the detection of false positives and thus reduce background. Such optimization permits a model with greater sensitivity that can identify additional true positive cancer samples unobscured by high background.

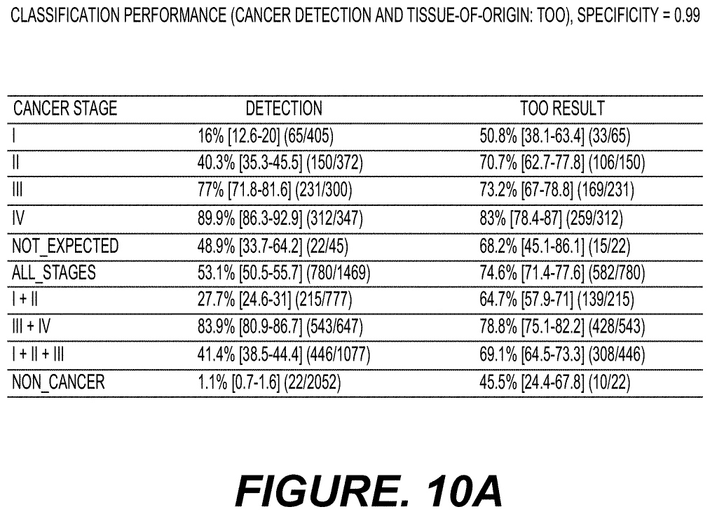

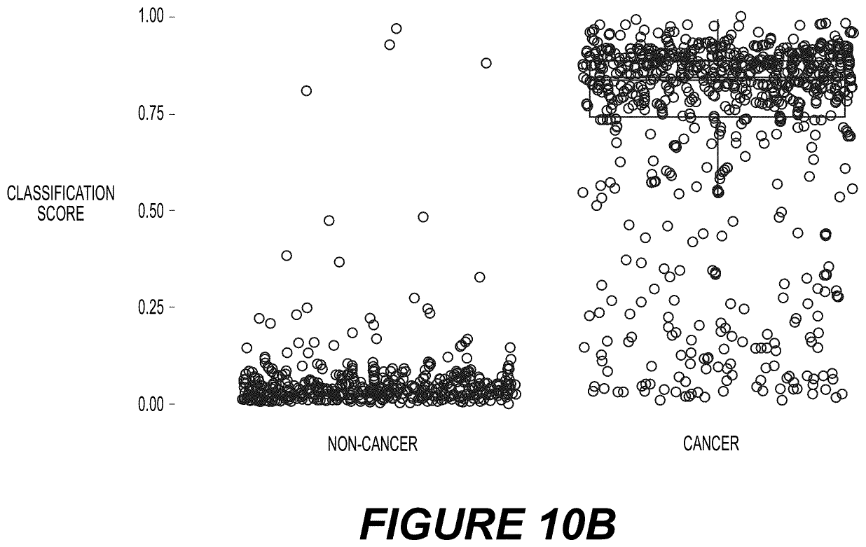

The performance of a Patch-CNN classifier was assessed for a panel of cancer samples grouped by cancer stage, as illustrated in FIG. 10A. Detection of all cancer samples was performed at 99% specificity. In one example, the sensitivity of detection (cancer versus non-cancer) for all cancer samples was 42.1%, the sensitivity of tissue-of-origin classifications for all cancer samples was 89.7%, and detection of early stage cancer samples was relatively low compared to late stage cancer samples (stage I: 10.1%, stage II: 29%, stage III: 58.3%, stage IV: 79.8%), although for each group of cancer stages the accuracy of tissue-of-origin predictions was high (approximately 90% sensitivity). FIG. 10B shows the performance of a Patch-CNN classifier in a binary setting (e.g., where samples are not categorized into 3 or more labels such as tissue of origin or stage). In this example, samples were classified as cancer or non-cancer. In a binary setting, the Patch-CNN classifier assigned non-cancer samples a mean probability of less than 10% and assigned cancer samples a mean probability of about 80%, indicating high performance of the binary classifier. Adjusting the parameters for 98%, 99%, and 99.5% specificity for the Patch-CNN classifier results in 88% sensitivity, 74.36% sensitivity, and 44.23% sensitivity, respectively.

### Example 3: Performance Testing by Isomap Clustering

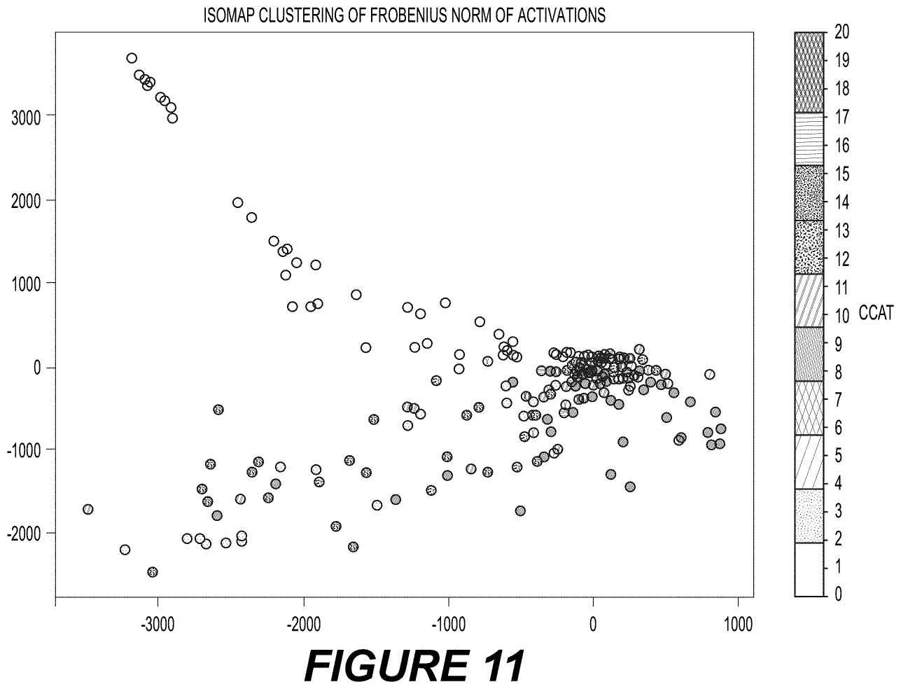

Referring to FIG. 11, a dimensional reduction technique was used to evaluate the performance of the embedding values (activations) generated following training for a patch-CNN classifier of the present disclosure, where activation refers to the ability of the embedding values to predict a classification for a sample. A set of cancer samples denoted by the labels 0 to 20 was used for classification. For each sample, features were extracted for each patch using a trained feature extractor. For each patch, the norm of the embedding values was calculated, and the norms for each patch within a given sample were concatenated to give a sample feature. The concatenated norms for each sample were then plotted by projection onto a manifold space. Specifically, a nonlinear dimensionality reduction method Isomap was used to cluster the different cancer labels within an N-dimensional space. The x and y-axes in the 2-dimensional coordinate space shown in FIG. 11 indicate relative distances between samples after clustering. The projections reveal that different cancer labels cluster to different regions of the Isomap, indicating that the embedding values are able to discriminate between samples with different labels. These results also suggest that either the embedding values or the norm of the embedding values can be used to provide information on performance.

### Example 4: Performance Testing by Patch Frequency of Maximal Activations

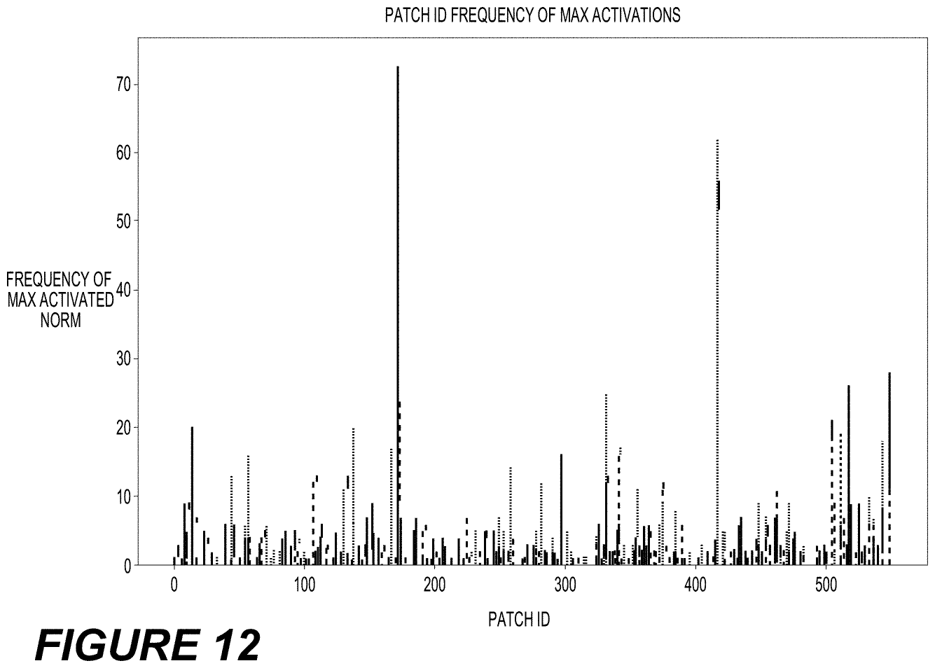

Referring to FIG. 12, a set of samples was evaluated using a patch-CNN model of the present disclosure that consisted of 544 patches, where each of the 544 patches represented a different portion of the human genome. For each of the 544 patches, the frequency of activations was determined across the set of samples. Thus, for example, if patch 10 of the 544 patches activated for samples 2 and 10 in the set of the samples, the y-value in FIG. 12 for patch 10 (X=10 in FIG. 12) would be 2. Specifically, a patch in the set of 544 patches incurring the highest signal to predict classifications for a sample was considered the maximally activated patch (e.g., where the embedding values are the most discriminative). For each patch in the set of 544 patches, the frequency of activation was calculated by determining the number of times that the respective patch was maximally activated compared to all other patches. FIG. 12 illustrates that most of the performance is derived from about 20 of the 544 patches, and that two patches in particular are highly indicative. Thus, some patches in the set of 544 patches activate more frequently than others and such patches likely drive classifier performance. For example, certain patches can specialize for different classification types (e.g., cancer and/or non-cancer). Furthermore, patch IDs that are highly indicative are likely to include CpG sites that are highly differential, providing a method to assess and optimize patch selection (e.g., to minimize the set of patches thus improving computational efficiency and/or reducing cost). Specifically, performance indicators as illustrated in FIG. 12 can guide a trained feature extractor model in bootstrapping a new region selection algorithm.

### Example 5: Performance Testing by t-SNE Clustering

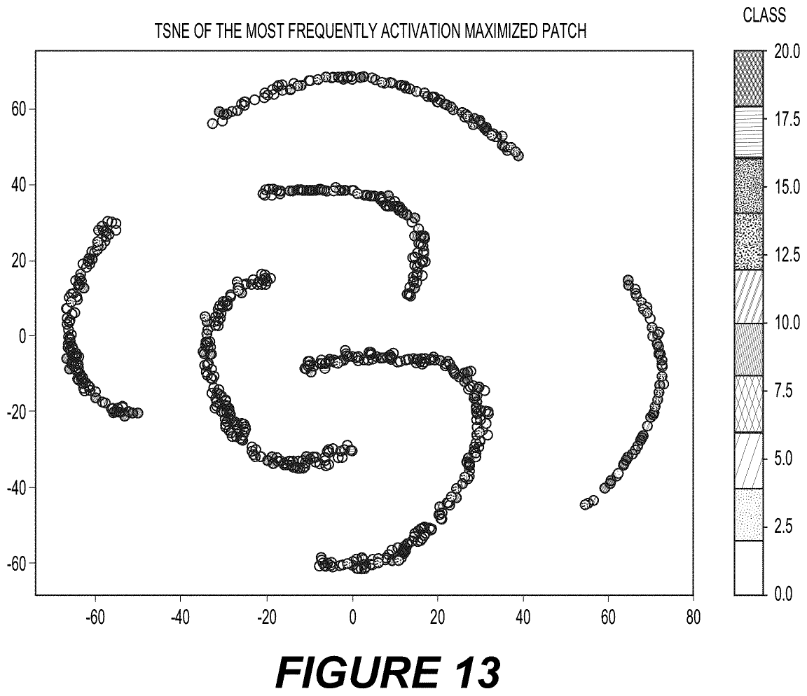

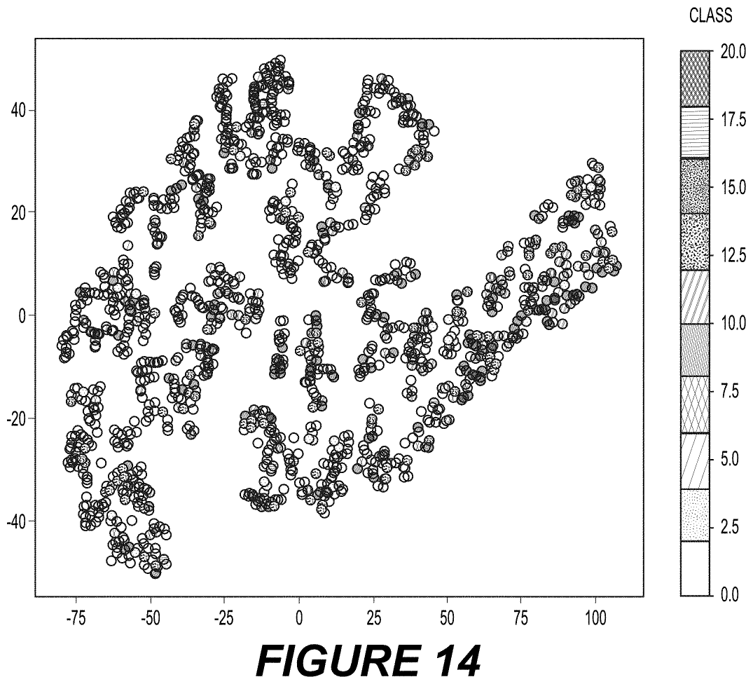

Referring to FIGS. 13 and 14, t-SNE clustering was performed for a set of samples using the embedding values for the top six (FIG. 13) or top three (FIG. 14) maximally activated patches. As described above in Example 4, maximally activated patches are those with the highest frequency of activations (e.g., the ability of a given patch to predict classifications for a given sample over all other patches). T-SNE clustering then performs a dimensional reduction and projects the data onto a 2-dimensional space. The set of 20 samples is indicated by the legend on the right where samples labels are denoted by 0 to 20, and each discrete point on the graph corresponds to a fragment of a sample. In FIG. 13, each cluster of points corresponds to one of the top six maximally activated patches. The cluster on the right hand side of FIG. 13 comprises mainly cancer samples, indicating that the patch represented by the respective cluster is capable of discriminating several different cancer types. This result parallels the observation from FIG. 12 that patches are unequally weighted during classification (e.g., some patches drive classification more than others). In FIG. 14, although t-SNE clustering of the top three maximally activated patches does not result in discrete clusters, there is a visible concentration of cancer types along the right hand side of the graph.

### Example 6: Performance Testing by Cancer Stage

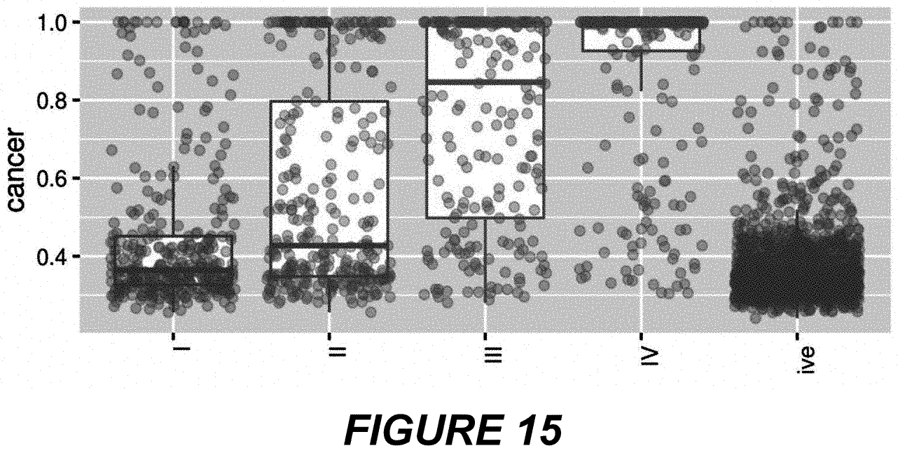

Referring to FIG. 15, classification performance using patch-CNN architecture of the present disclosure was compared for stages I, II, III and IV of cancer samples. Data was obtained from a subset of the Circulating Cell-free Genome Atlas study (CCGA 2) and filtered for 98% specificity. Resulting sensitivity of the data set was 45% for the model. Classification scores are presented along the y-axis, where 0 denotes non-cancer and 1 denotes cancer. Each discrete point represents a sample (e.g., an individual subject). Non-informative samples are included as a reference on the right side of the graph. FIG. 15 illustrates that classification performance improves with progressive cancer stages, such that stage I cancer samples are assigned a mean probability of less than 0.4 that the subject has cancer, while stage IV cancer samples are assigned a mean probability of 1 that the subject has cancer.

### Example 7: Performance Testing by Tissue of Origin

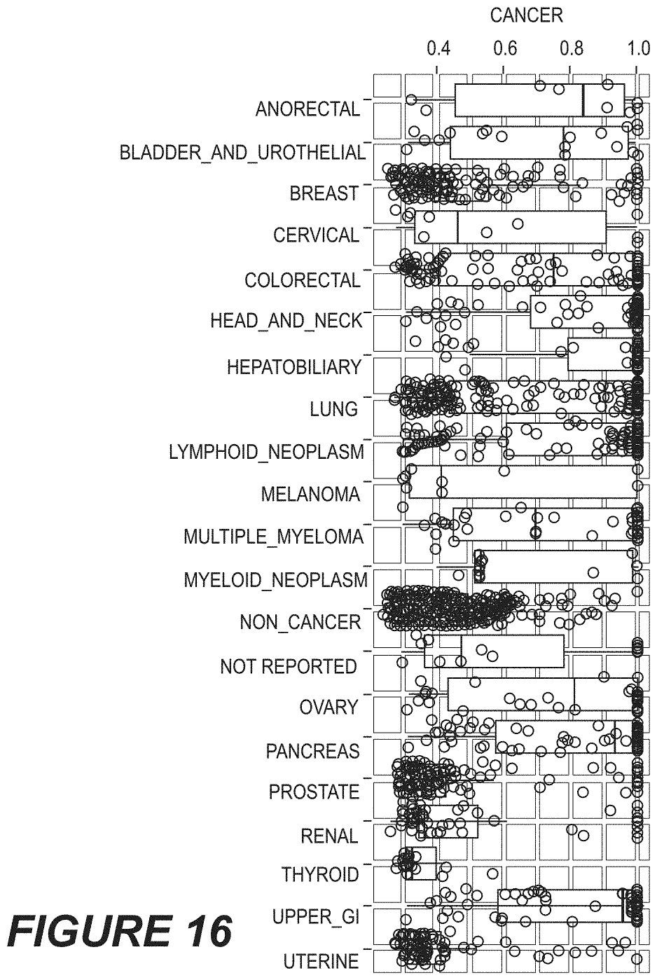

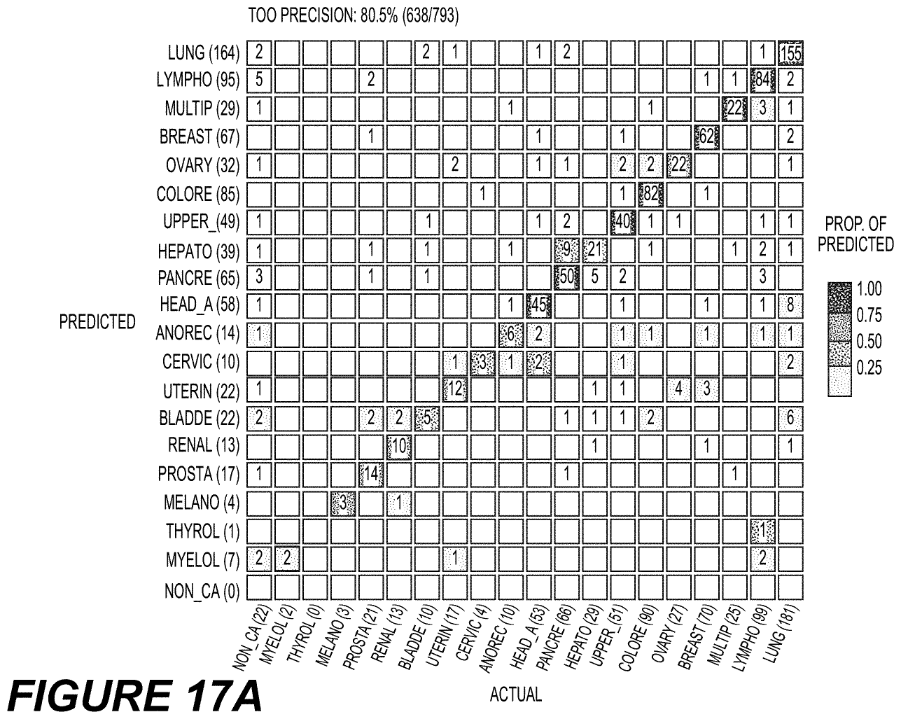

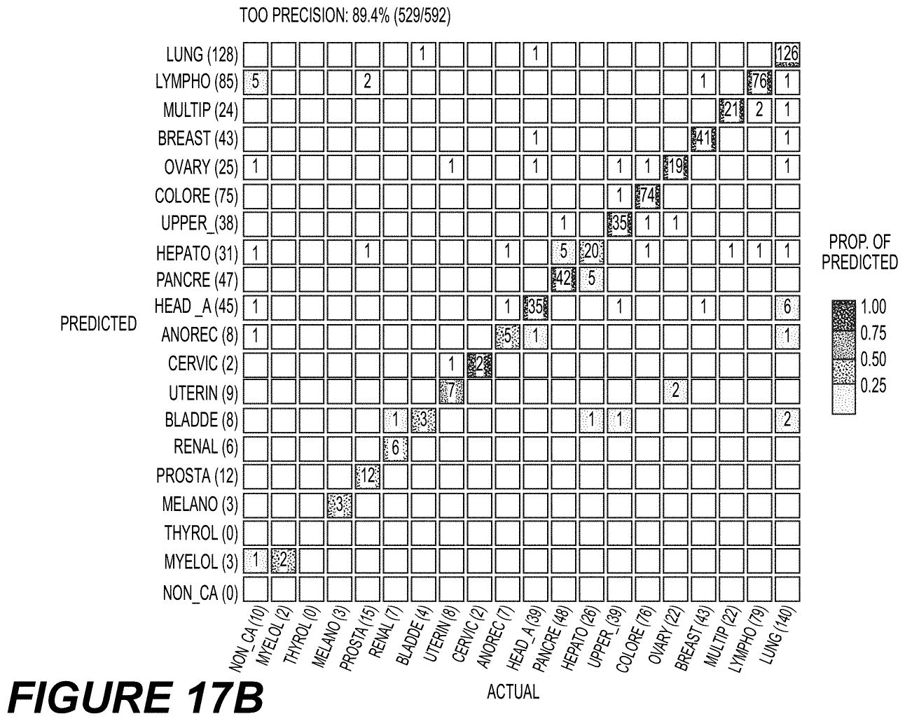

Referring to FIGS. 16, 17A and 17B, classification performance using a patch-CNN architecture of the present disclosure was evaluated for samples originating from a variety of tissue origins. Data was obtained from CCGA 2. In FIG. 16, classification scores are presented along the y-axis, where 0 denotes non-cancer and 1 denotes cancer. Each discrete point represents a sample (e.g., an individual subject). Interestingly, classification results for individual cancer types were consistent between CCGA 1 and CCGA 2 datasets. Eleven high-signal cancer types were identified as easily detectable (e.g., probability of greater than 0.6) compared to other cancer types, including anorectal, bladder and urothelial, colorectal, head and neck, hepatobiliary, lung, lymphoid neoplasm, multiple myeloma, ovary, pancreas, and upper GI.

FIGS. 17A and 17B illustrates results of confusion matrix analysis performed using a “take one out” method for tissue of origin in which above 80 percent accuracy for predictions was achieved without indeterminate analysis (FIG. 17A) and about 90 percent accuracy for predictions was achieved with indeterminate analysis (FIG. 17B).

Specifically, in FIG. 17A, lymphoid neoplasm cancer samples were correctly classified with 84% accuracy (84/99) and lung cancer samples were correctly classified with 86% accuracy (155/181). Other high-signal cancer types were predicted with varying degrees of accuracy including breast (62/70 at 89%), colorectal (82/90 at 91%), head and neck (45/53 at 85%), hepatobiliary (21/29 at 72%), multiple myeloma (22/25 at 88%), ovary (22/27 at 81%), pancreas (50/66 at 76%), and upper GI (40/51 at 78%).

In FIG. 17B, removal of indeterminate samples further enhanced tissue of origin classification. Lymphoid neoplasm cancer samples were correctly classified with 96% accuracy (76/79) and lung cancer samples were correctly classified with 98.4% accuracy (126/140). Other high-signal cancer types were predicted with varying degrees of accuracy including breast (41/43 at 95%), colorectal (74/76 at 97%), head and neck (35/39 at 90%), hepatobiliary (20/26 at 77%), multiple myeloma (21/22 at 95%), ovary (19/22 at 86%), pancreas (42/48 at 88%), and upper GI (35/39 at 90%).

### Example 8: Encoding Hyperparameters

Hyperparameters for the disclosed patch CNN classifiers were encoded and defined. The use of such hyperparameters allowed the patch CNN classifiers of the present disclosure to be rapidly tuned and adjusted to accommodate and/or optimize different types of experimental designs, applications, sequencing methods, stringencies, accuracies, and/or computational attributes, among others. Examples of adjustable hyperparameters included number of patches (e.g., between 10 and 1000 patches), number of CpG sites evaluated per patch (e.g., image width such as between 10 and 1000 CpG sites or between 64 and 512 CpG sites, image width such as 128 CpG sites or 256 CpG sites), depth of fragments per patch (e.g., image height such as between 2 and 1000 fragments, or image height such as 32, 50, 64, or 128 fragments), density of fragment packing within a patch, which packing algorithm is used to position nucleic acid fragments within a patch, among others. Additional example hyperparameters included, but are not limited to, p-value (the value used to prune the input plurality of nucleic acid fragments by removing from the plurality of nucleic acid fragments each respective nucleic acid fragment whose corresponding methylation pattern when evaluated against corresponding nucleic acid fragments in a cohort failed to satisfy a p-value threshold set by the p-value hyperparameter such as p=0.05 or p=0.001), type of cross validation used (e.g., P×Q-fold cross-validation, where P and Q were positive integers and were the same or different as described herein), L2 regularization dropout rate (e.g., 0.250000), L2 regularization initial learning rate (e.g., 0.000200), and L2 regularization factor (e.g., 0.010000). A loss function for such regularization was performed over a number of cycles and the performance of the classifier for each set of hyperparameters was evaluated using metrics for sensitivity, specificity, and accuracy.

### Example 9: Creating and Validating Control Data Structures for Quality Control

As described above, FIGS. 3 and 4 illustrate workflows used for the classification of cancer conditions from methylation sequencing data. Quality control and/or quality monitoring was performed on the data after the initial pre-processing and prior to methylation calling and p-value-based pruning. A control group was used to compare a test sample (e.g., cancer) to a data structure comprising normal or healthy sample data. An example workflow for generating a data structure for a healthy control group is described herein. To create a healthy control group data structure, an analytics system (or a processing system described elsewhere herein) received a plurality of nucleic acid fragments (e.g., cfDNA) from a plurality of subjects. A set of methylation state vectors were generated for the control group by identifying a methylation state vector for each nucleic acid fragment.

With each nucleic acid fragment's methylation state vector, the analytics system subdivided the methylation state vector into strings of methylation sites (e.g., CpG sites). The analytics system subdivided the methylation state vector such that the resulting strings were all less than a given length. For example, a methylation state vector of length 11 subdivided into strings of length less than or equal to 3 resulted in 9 strings of length 3, 10 strings of length 2, and 11 strings of length 1. In another example, a methylation state vector of length 7 subdivided into strings of length less than or equal to 4 resulted in 4 strings of length 4, 5 strings of length 3, 6 strings of length 2, and 7 strings of length 1. If a methylation state vector was shorter than or the same length as the specified string length, then the methylation state vector was converted into a single string containing all of the CpG sites of the vector.

The analytics system tallied the strings by counting, for each possible CpG site and possibility of methylation states in the vector, the number of strings present in the control group having the specified CpG site as the first CpG site in the string and having that possibility of methylation states. For example, at a given CpG site and considering string lengths of 3, there were 2³ or 8 possible string configurations. At that given CpG site, for each of the 8 possible string configurations, the analytics system tallied how many occurrences of each methylation state vector possibility came up in the control group. Continuing this example, this involved tallying the following quantities: <Mₓ, Mₓ₊₁, Mₓ₊₂>, <Mₓ, Mₓ₊₁, Uₓ₊₂>, . . . , <Uₓ, Uₓ₊₁, Uₓ₊₂> for each starting CpG site x in the reference genome. The analytics system created the data structure storing the tallied counts for each starting CpG site and string possibility.

There are several benefits to setting an upper limit on string length. First, depending on the maximum length for a string, the size of the data structure created by the analytics system can dramatically increase in size. For instance, maximum string length of 4 means that every CpG site has at the very least 2⁴ numbers to tally for strings of length 4. Increasing the maximum string length to 5 means that every CpG site has an additional 2⁴ or 16 numbers to tally, doubling the numbers to tally (and computer memory) compared to the prior string length. Reducing string size helps keep the data structure creation and performance (e.g., use for later accessing as described below), in terms of computational and storage, reasonable. Second, a statistical consideration to limiting the maximum string length is to avoid overfitting downstream models that use the string counts. If long strings of CpG sites do not, biologically, have a strong effect on the outcome (e.g., predictions of anomalousness that predictive of the presence of cancer), calculating probabilities based on large strings of CpG sites can be problematic as it uses a significant amount of data that may not be available, and thus can be too sparse for a model to perform appropriately. For example, calculating a probability of anomalousness/cancer conditioned on the prior 100 CpG sites can utilize counts of strings in the data structure of length 100, ideally some matching exactly the prior 100 methylation states. If sparse counts of strings of length 100 are available, there can be insufficient data to determine whether a given string of length of 100 in a test sample is anomalous or not.

Once the data structure was created, the analytics system sought to validate the data structure and/or any downstream models making use of the data structure. One type of validation checked consistency within the control group's data structure. For example, if there were any outlier subjects, samples, and/or fragments within a control group, then the analytics system performed various calculations to determine whether to exclude any fragments from one of those categories. In a representative example, the healthy control group contained a sample that was undiagnosed but cancerous such that the sample contained anomalously methylated fragments. This first type of validation ensured that potential cancerous samples were removed from the healthy control group so as to not affect the control group's purity.

A second type of validation checked the probabilistic model used to calculate p-values with the counts from the data structure itself (i.e., from the healthy control group). Once the analytics system generated a p-value for the methylation state vectors in the validation group, the analytics system built a cumulative density function (CDF) with the p-values. With the CDF, the analytics system performed various calculations on the CDF to validate the control group's data structure. One test used the fact that the CDF was ideally at or below an identity function, such that CDF(x)≤x. On the converse, being above the identity function revealed some deficiency within the probabilistic model used for the control group's data structure. For example, if 1/100 of fragments had a p-value score of 1/1000 meaning CDF( 1/1000)= 1/100> 1/1000, then the second type of validation failed indicating an issue with the probabilistic model.

A third type of validation used a healthy set of validation samples separate from those used to build the data structure, which tested if the data structure was properly built and the model worked. The third type of validation quantified how well the healthy control group generalized the distribution of healthy samples. If the third type of validation failed, then the healthy control group did not generalize well to the healthy distribution. A fourth type of validation tested with samples from a non-healthy validation group.

The analytics system calculated p-values and builds the CDF for the non-healthy validation group. With a non-healthy validation group, the analytics system saw the CDF(x)>x for at least some samples or, stated differently, the converse of what was expected in the second type of validation and the third type of validation with the healthy control group and the healthy validation group. If the fourth type of validation failed, then this was indicative that the model was not appropriately identifying the anomalousness that it was designed to identify.

An additional workflow was performed in order to validate the consistency of the control group data structure. The analytics system utilized a validation group with a supposedly similar composition of subjects, samples, and/or fragments as the control group. For example, if the analytics system selected healthy subjects without cancer for the control group, then the analytics system also used healthy subjects without cancer in the validation group.

The validation workflow comprised generating a set of methylation state vectors for the validation group as described for the control group. For each methylation state vector, all possible methylation state vectors at that position were enumerated, and the probabilities of all possible methylation state vectors from the control group data structure were calculated. A p-value score was then calculated for each methylation state vector based on the calculated probabilities, and a cumulative density function (CDF) of all p-values from the validation group was generated. The p-value score represented an expectedness of finding that specific methylation state vector and other possible methylation state vectors having even lower probabilities in the control group. A low p-value score, therefore, corresponded to a methylation state vector which was relatively unexpected in comparison to other methylation state vectors within the control group, where a high p-value score corresponded to a methylation state vector which was relatively more expected in comparison to other methylation state vectors found in the control group. Using the CDF, the consistency of the p-values within the data structure of the control group was validated.

### Example 10: Determining Metastasis Disease Statuses

Table 3 shows some examples of using cfDNA fragments in plasma samples from cancer patients afflicted with metastases to determine metastasis disease statuses. The determination of metastatic processes was performed with the same classifier that was used to detect the presence of cancer and tissues of origin (TOO).

For example, the TOO reference dataset included plasma samples from 18 subjects with pancreatic cancers and a known metastasis to the liver. Out of these 18 subjects, signals from the liver were seen in plasma samples in 9 subjects. However, signals from the liver were also seen in plasma samples from remaining subjects with pancreatic cancer, but the signal was less common. Similarly, as another example, the TOO reference dataset included plasma samples from 4 subjects with breast cancers and known metastases to lung, brain, bone, and liver. The samples with metastases to brain and bone had strong cross-scores (e.g., normalized probabilities of cancer) for tissues of origin other than breast, even if no classes represented brain tissue for the trained classifier. Also, the cross-scores for the sample with bone metastases included scores for multiple myeloma and sarcoma with a methylation signal similar to those of some cells in the bone marrow.

In another example, the TOO reference dataset included plasma samples from 13 subjects with lung cancers and known metastases to bone, brain, pericardium, and liver. The samples with metastases to bone and brain had strong cross-scores (e.g., normalized probabilities of cancer) for tissues other than lung. In a further example, the TOO reference dataset included plasma samples from 10 subjects with colorectal cancers and a known metastasis to liver. There was no clearly visible methylation signal from liver cells in samples from the subjects with colorectal cancer and metastases to the liver.

TABLE 3
TOO results (e.g., normalized probabilities of cancer)
for different subjects with different primary cancers.

| Subject | Primary | Site of | Tissue-of-origin |
|---|---|---|---|
| Sample ID# | cancer | metastases | classification results |
| 8024414998 | pancreas | bile duct | 0.227 hepatobiliary_biliary |
| | | | 0.098 pancreas |
| | | | 0.095 hepatobiliary_hcc |
| | | | 0.080 renal_urothelial |
| | | | 0.056 cervical |
| | | | 0.054 neuroendocrine |
| | | | 0.043 anorectal |
| | | | 0.043 sarcoma |
| | | | 0.035 upper_gi_all_other |
| | | | 0.033 melanoma |
| | | | 0.030 bladder |
| | | | 0.027 head_neck |
| | | | 0.024 colorectal |
| | | | 0.022 upper_gi_squamous |
| | | | 0.022 multiple_myeloma |
| | | | 0.022 renal_all_other |
| | | | 0.019 thyroid |
| | | | 0.016 ovarian |
| | | | 0.015 lung_sclc |
| 8024619221 | pancreas | liver | 0.967 hepatobiliary_biliary |
| 8024414084 | pancreas | liver | 0.516 pancreas |
| | | | 0.112 hepatobiliary_biliary |
| | | | 0.061 upper_gi_all_other |
| | | | 0.044 cervical |
| | | | 0.034 anorectal |
| | | | 0.024 sarcoma |
| | | | 0.024 neuroendocrine |
| | | | 0.023 upper_gi_squamous |
| | | | 0.017 renal_urothelial |
| | | | 0.015 ovarian |
| | | | 0.015 hepatobiliary_hcc |
| | | | 0.013 multiple_myeloma |
| | | | 0.013 melanoma |
| | | | 0.012 colorectal |
| 8024576490 | pancreas | liver | 0.946 pancreas |
| | | | 0.040 hepatobiliary_biliary |
| 8024621468 | pancreas | liver | 0.996 pancreas |
| 8024622168 | pancreas | liver | 0.787 pancreas |
| | | | 0.100 hepatobiliary_biliary |
| | | | 0.022 lung_all_other |
| | | | 0.019 renal_urothelial |
| | | | 0.011 neuroendocrine |
| 8024605732 | pancreas | liver | 0.974 pancreas |
| | | | 0.014 hepatobiliary_biliary |
| 8024615058 | pancreas | liver | 0.940 pancreas |
| | | | 0.052 upper_gi_all_other |
| 8024420827 | pancreas | liver | 0.979 pancreas |
| | | | 0.016 hepatobiliary_biliary |
| 8024423080 | pancreas | liver | 0.997 pancreas |
| 8025990127 | pancreas | liver | 0.991 pancreas |
| 8024927748 | pancreas | liver | 0.873 pancreas |
| | | | 0.042 upper_gi_all_other |
| | | | 0.022 neuroendocrine |
| | | | 0.013 anorectal |
| | | | 0.011 hepatobiliary_biliary |
| 8024623662 | pancreas | liver | 0.993 pancreas |
| 8024581812 | pancreas | liver | 0.974 pancreas |
| 8024414859 | pancreas | liver | 0.744 pancreas |
| | | | 0.096 upper_gi_all_other |
| | | | 0.049 neuroendocrine |
| | | | 0.029 head_neck |
| | | | 0.021 upper_gi_squamous |
| | | | 0.019 lung_sclc |
| | | | 0.015 anorectal |
| 8024425726 | pancreas | liver | 0.460 pancreas |
| | | | 0.115 renal_urothelial |
| | | | 0.050 lung_adenocarcinoma |
| | | | 0.041 bladder |
| | | | 0.040 hepatobiliary_biliary |
| 8024619637 | pancreas | liver | 0.678 pancreas |
| | | | 0.195 upper_gi_all_other |
| | | | 0.027 neuroendocrine |
| | | | 0.014 lung_adenocarcinoma |
| | | | 0.013 renal_urothelial |
| | | | 0.011 hepatobiliary_biliary |
| 8024422200 | pancreas | lung | 0.837 pancreas |
| | | | 0.051 hepatobiliary_biliary |
| | | | 0.022 renal_urothelial |
| | | | 0.021 sarcoma |
| | | | 0.015 neuroendocrine |
| 8024419981 | breast | bone* | 0.863 breast |
| | | | 0.016 multiple_myeloma |
| | | | 0.014 sarcoma |
| | | | 0.013 pancreas |
| 8023556702 | breast | brain* | 0.596 breast |
| | | | 0.060 lung_adenocarcinoma |
| | | | 0.046 sarcoma |
| | | | 0.041 hepatobiliary_biliary |
| | | | 0.032 renal_urothelial |
| | | | 0.025 neuroendocrine |
| | | | 0.025 upper_gi_squamous |
| | | | 0.025 hepatobiliary_hcc |
| | | | 0.021 upper_gi_all_other |
| | | | 0.020 lung_all_other |
| 8025741820 | breast | liver | 0.996 breast |
| 8024613251 | breast | lung | 0.996 breast |
| 8024416247 | lung | bone* | 0.917 lung_adenocarcinoma |
| | | | 0.040 lung_all_other |
| | | | 0.020 breast |
| 8024611491 | lung | bone* | 0.999 lung_adenocarcinoma |
| 8024612498 | lung | bone* | 0.111 lung_adenocarcinoma |
| | | | 0.074 renal_urothelial |
| | | | 0.072 sarcoma |
| | | | 0.069 upper_gi_squamous |
| | | | 0.067 neuroendocrine |
| | | | 0.054 melanoma |
| | | | 0.051 thyroid |
| | | | 0.050 non_cancer |
| | | | 0.042 multiple_myeloma |
| | | | 0.041 anorectal |
| | | | 0.041 hepatobiliary_hcc |
| | | | 0.041 pancreas |
| | | | 0.038 head_neck |
| | | | 0.037 hepatobiliary_biliary |
| | | | 0.027 cervical |
| | | | 0.026 upper_gi_all_other |
| | | | 0.023 lung_all_other |
| | | | 0.022 leukemia |
| | | | 0.019 breast |
| | | | 0.019 lung_sclc |
| | | | 0.019 uterine |
| | | | 0.016 lymphoma |
| | | | 0.012 bladder |
| | | | 0.012 prostate |
| | | | 0.010 ovarian |
| 8024608645 | lung | bone* | 0.969 lung_adenocarcinoma |
| | | | 0.020 lung_all_other |
| 8024620380 | lung | bone* | 1 lung_adenocarcinoma |
| 8025758541 | lung | brain* | 0.846 lung_adenocarcinoma |
| | | | 0.035 hepatobiliary_biliary |
| | | | 0.022 renal_urothelial |
| | | | 0.015 neuroendocrine |
| | | | 0.014 sarcoma |
| | | | 0.010 thyroid |
| 8023555042 | lung | brain* | 0.287 lung_adenocarcinoma |
| | | | 0.120 sarcoma |
| | | | 0.066 neuroendocrine |
| | | | 0.062 renal_all_other |
| | | | 0.056 cervical |
| | | | 0.052 hepatobiliary_hcc |
| | | | 0.047 melanoma |
| | | | 0.039 thyroid |
| | | | 0.036 anorectal |
| | | | 0.034 lung_all_other |
| | | | 0.033 lung_sclc |
| | | | 0.024 upper_gi_all_other |
| | | | 0.023 leukemia |
| | | | 0.021 hepatobiliary_biliary |
| | | | 0.021 head_neck |
| | | | 0.017 pancreas |
| | | | 0.016 renal_urothelial |
| | | | 0.012 breast |
| 8024618117 | lung | pericardium | 0.992 lung_adenocarcinoma |
| 8024610687 | lung | peritoneum | 0.995 lung_adenocarcinoma |
| 8025757681 | lung | unknown | 0.955 lung_adenocarcinoma |
| | | | 0.011 lung_all_other |
| 8024612601 | lung | liver | 0.831 lung_all_other |
| | | | 0.051 head_neck |
| | | | 0.035 upper_gi_squamous |
| | | | 0.028 cervical |
| | | | 0.023 renal_urothelial |
| | | | 0.011 lung_adenocarcinoma |
| 8024621483 | lung | liver\|peritoneum | 0.941 lung_all_other |
| | | | 0.025 head_neck |
| | | | 0.013 renal_urothelial |
| | | | 0.010 upper_gi_squamous |
| 8024623924 | lung | pelvis | 0.930 lung_all_other |
| | | | 0.035 upper_gi_squamous |
| 8024610695 | colorectal | liver | 0.991 colorectal |
| 8024620611 | colorectal | liver | 0.996 colorectal |
| 8024611930 | colorectal | liver | 0.999 colorectal |
| 8025752253 | colorectal | liver | 1 colorectal |
| 8024623340 | colorectal | liver | 0.999 colorectal |
| 8024413579 | colorectal | liver | 1 colorectal |
| 8024613608 | colorectal | liver | 0.999 colorectal |
| 8024576685 | colorectal | liver | 0.998 colorectal |
| 8026589395 | colorectal | liver | 1 colorectal |
| 8024618429 | colorectal | ovaries | 0.120 colorectal |
| | | | 0.115 renal_urothelial |
| | | | 0.074 hepatobiliary_biliary |
| | | | 0.072 neuroendocrine |
| | | | 0.064 anorectal |
| | | | 0.063 sarcoma |
| | | | 0.055 bladder |
| | | | 0.047 leukemia |
| | | | 0.045 upper_gi_squamous |
| | | | 0.042 upper_gi_all_other |
| | | | 0.040 hepatobiliary_hcc |

### CONCLUSION

Plural instances may be provided for components, operations or structures described herein as a single instance. Boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event (” or “in response to detecting (the stated condition or event),” depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purposes of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

1. A method of determining a cancer condition of a test subject of a species, the method comprising:
at a computer system comprising at least one processor and a memory storing at least one program for execution by the at least one processor, the at least one program comprising instructions for:
A) obtaining a dataset, in electronic form, wherein the dataset comprises a corresponding methylation pattern of each respective fragment in a plurality of fragments, wherein the corresponding methylation pattern of each respective fragment (i) is determined by a methylation sequencing of one or more nucleic acid samples comprising the respective fragment in a biological sample obtained from the test subject and (ii) comprises a methylation state of each CpG site in a corresponding plurality of CpG sites in the respective fragment;
B) constructing a first patch comprising a first channel, the first patch representing a first independent set of CpG sites in a reference genome of the species, each respective CpG site in the first independent set of CpG sites corresponding to a predetermined location in the reference genome, wherein:
the first channel of the first patch comprises a plurality of instances of a first plurality of parameters, wherein each instance of the first plurality of parameters includes a parameter for a methylation status of a respective CpG site in the first independent set of CpG sites for the first patch,
the constructing B) comprises populating, for each respective fragment in the plurality of fragments that aligns to the first independent set of CpG sites, an instance of all or a portion of the first plurality of parameters based on the methylation pattern of the respective fragment; and
C) applying at least the first patch to a classifier thereby determining the cancer condition in the test subject.

2. The method of claim 1, wherein the at least one program further comprises instructions for, after the obtaining A) and prior to the constructing B):
pruning the plurality of fragments by removing from the plurality of fragments each respective fragment, whose corresponding methylation pattern across a corresponding plurality of CpG sites in the respective fragment, has a p-value that fails to satisfy a p-value threshold, wherein the p-value of the respective fragment is determined based upon a comparison of the corresponding methylation pattern of the respective fragment to a corresponding distribution of methylation patterns of the corresponding plurality of CpG sites in a corresponding plurality of reference fragments that have the corresponding plurality of CpG sites of the respective fragment, wherein the methylation pattern of each reference fragment in the corresponding plurality of reference fragments is obtained by a methylation sequencing of nucleic acid from biological samples obtained from a cohort of healthy subjects.

3. The method of claim 1, wherein:
the first patch comprises a plurality of channels including the first channel and a second channel,
the second channel comprises a corresponding instance of a second plurality of parameters for each instance of the first plurality of parameters, wherein each instance of the second plurality of parameters includes a parameter for a first characteristic, other than CpG methylation state, of a respective CpG site in the first independent set of CpG sites for the first patch, and
the constructing B) comprises populating, for each respective fragment in the plurality of fragments that aligns to the first independent set of CpG sites, an instance of all or a portion of the first plurality of parameters and an instance of all or a portion of the second plurality of parameters based on the methylation pattern of the respective fragment.

4. The method of claim 1, wherein the methylation pattern of a respective fragment does not include each CpG site in the first independent set of CpG sites of the first patch and wherein the constructing B), for a respective fragment in the plurality of fragments, comprises populating parameters in the instance of first plurality of parameters that correspond to CpG sites present in the respective fragment.

5. The method of claim 1, wherein the constructing B), for a respective fragment in the plurality of fragments, comprises:
i) identifying, within an instance of the first plurality of parameters of the first channel, parameters, corresponding to the CpG sites in the respective fragment, that have not previously been assigned methylation states based on another fragment in the plurality of fragments; and
ii) assigning for each parameter, among the identified parameters, that aligns to a corresponding CpG site of the respective fragment, the methylation state of the corresponding CpG site of the respective fragment.

6. The method of claim 3, wherein the constructing B), for a respective fragment in the plurality of fragments, comprises:
i) identifying, within an instance of the first plurality of parameters of the first channel, parameters, corresponding to the CpG sites in the respective fragment, that have not previously been assigned methylation states based on another fragment in the plurality of fragments;
ii) assigning for each parameter, among the identified parameters, that aligns to a respective CpG site of the respective fragment, the methylation state of the respective CpG site of the respective fragment; and
iii) assigning for each parameter, among the identified parameters, in the second plurality of parameters of the instance of the second plurality of parameters of the second channel that corresponds to the instance of the first plurality of parameters, that aligns to a respective CpG site of the respective fragment, the first characteristic of the respective CpG site of the respective fragment.

7. (canceled)

8. The method of claim 6, wherein the first characteristic of the respective CpG site is selected from the group consisting of:
a CpG β-value drawn from a healthy cohort,
a CpG β-value drawn from a predetermined tissue type in a healthy cohort,
a CpG β-value drawn from the test subject,
a Pearson's correlation score for methylation state of 5′ and 3′ neighbor CpG sites,
a Jaccard similarity, Euclidean distance, Manhattan distance, maximum value, normalized Euclidean distance, normalized maximum value, dice coefficient, or cosine coefficient of methylation state of the respective CpG site in the test subject versus a cohort of subjects,
a fragment p-value of the respective fragment,
a length of the respective fragment the respective CpG site is on,
a fragment sequence source,
a fragment mapping quality score of the respective fragment the respective CpG site is on,
a distance to a 5′ adjacent CpG site in the reference genome,
a distance to a 3′ adjacent CpG site in the reference genome,
a multiplicity of the respective fragment the respective CpG site is on,
a genetic element the respective CpG site is within,
a biological pathway the respective CpG site is associated with,
a gene the respective CpG site is associated with,
a value of a CpG transition impulse function for the respective CpG site,
a value of a CpG run-length encoding for the respective CpG site, and
a read strand orientation of the fragment the respective CpG site is on.

9. The method of claim 5, wherein more than one fragment in the plurality of fragments is assigned to a single instance of the first plurality of parameters of the first channel in the first patch provided that the more than one fragment does not have common CpG sites.

10. The method of claim 4, wherein parameters in the instance of the first plurality of parameters are zero filled.

11. The method of claim 1, wherein the first independent set of CpG sites are in a CpG index of the reference genome, and wherein the CpG index of the reference genome includes a first CpG site, not present in the first independent set of CpG sites, that is located in the reference genome between a second CpG site and a third CpG site that are present in the first independent set of CpG sites.

12. (canceled)

13. The method of claim 1, wherein:
the first independent set of CpG sites includes a first CpG site and a second CpG site that are adjacent to each other in a CpG index of the reference genome,
a first fragment in the plurality of fragments includes the first CpG site but not the second CpG site, and
a second fragment in the plurality of fragments includes the second CpG site but not the first CpG site.

14. The method of claim 1, wherein a parameter in an instance of the first plurality of parameters, for a respective fragment in the plurality of fragments, is:
methylated when the corresponding CpG site in the respective fragment is determined by the methylation sequencing to be methylated,
unmethylated when the corresponding CpG site in the respective fragment is determined by the methylation sequencing to not be methylated,
other when the corresponding CpG site in the respective fragment is determined by the methylation sequencing to be other than methylated or unmethylated.

15. (canceled)

16. (canceled)

17. (canceled)

18. The method of claim 3, wherein:
the plurality of channels comprises at least three channels; and
a third channel in the first plurality of channels comprises a corresponding instance of a third plurality of parameters for each instance of the first plurality of parameters, wherein each instance of the third plurality of parameters includes a parameter for a second characteristic of a respective CpG site in the first independent set of CpG sites, wherein the second characteristic is selected from the group consisting of:
a CpG β-value drawn from a healthy cohort,
a CpG β-value drawn from a predetermined tissue type in a healthy cohort,
a CpG β-value drawn from the test subject,
a Pearson's correlation score for methylation state of 5′ and 3′ neighbor CpG sites,
a Jaccard similarity, Euclidean distance, Manhattan distance, maximum value, normalized Euclidean distance, normalized maximum value, dice coefficient, or cosine coefficient of methylation state of the respective CpG site in the test subject versus a cohort of subjects,
a fragment p-value of the respective fragment,
a length of the respective fragment the respective CpG site is on,
a fragment sequence source,
a fragment mapping quality score of the respective fragment the respective CpG site is on,
a distance to a 5′ adjacent CpG site in the reference genome,
a distance to a 3′ adjacent CpG site in the reference genome,
a multiplicity of the respective fragment the respective CpG site is on,
a genetic element the respective CpG site is within,
a biological pathway the respective CpG site is associated with,
a gene the respective CpG site is associated with,
a value of a CpG transition impulse function for the respective CpG site,
a value of a CpG run-length encoding for the respective CpG site, and
a read strand orientation of the fragment the respective CpG site is on.

19. (canceled)

20. The method of claim 1, the at least one program further comprising instructions for:
constructing a second patch comprising a corresponding first channel, the second patch representing a second independent set of CpG sites in the reference genome of the species, each respective CpG site in the second independent set of CpG sites corresponding to a predetermined location in the reference genome, wherein the corresponding first channel of the second patch comprises a corresponding plurality of instances of a first plurality of parameters, wherein each instance of the corresponding first plurality of parameters of the second channel includes a parameter for a methylation status of a respective CpG site in the second independent set of CpG sites for the second patch; and
populating, for each respective fragment in the plurality of fragments that aligns to the second independent set of CpG sites, an instance of all or a portion of the first plurality of parameters of the second patch based on the methylation pattern of the respective fragment thereby constructing the second patch; and wherein
the applying C) further comprises applying the first and second patches to the classifier thereby determining the cancer condition in the test subject.

21. The method of claim 20, wherein:
the second patch comprises a corresponding plurality of channels including the corresponding first channel;
a corresponding second channel in the corresponding plurality of channels of the second patch comprises a corresponding instance of a second plurality of parameters for each instance of the first plurality of parameters, wherein each instance of the second plurality of parameters of the second patch includes a parameter for a first characteristic, other than CpG methylation state, of a respective CpG site in the second independent set of CpG sites for the second patch; and
the instructions for populating, for each respective fragment in the plurality of fragments that aligns to the second independent set of CpG sites, further populates an instance of all or a portion of the instance of the second plurality of parameters of the second patch based on the methylation pattern of the respective fragment.

22. (canceled)

23. (canceled)

24. (canceled)

25. The method of claim 20, wherein the first patch represents a first portion of the reference genome and the second patch represents a second portion of the reference genome, wherein a size of the first portion is different than a size of the second portion.

26. (canceled)

27. (canceled)

28. The method of claim 1, wherein the methylation sequencing of one or more nucleic acid samples is i) whole genome methylation sequencing or ii) targeted DNA methylation sequencing using a plurality of nucleic acid probes.

29. The method of claim 28, wherein the methylation sequencing of one or more nucleic acid samples uses a plurality of nucleic acid probes and the plurality of nucleic acid probes comprises one hundred or more probes.

30. (canceled)

31. (canceled)

32. (canceled)

33. (canceled)

34. The method of claim 1, wherein:
the at least one program further comprises instructions for constructing a plurality of patches including the first patch, each respective patch being for a different independent set of CpG sites in the reference genome;
the constructing B) constructs a plurality of patches including the first patch;
the classifier comprises a plurality of trained first stage models and a second stage model;
the applying the at least the first patch to a classifier comprises:
obtaining a feature vector comprising a plurality of feature elements, wherein each feature element in the plurality of feature elements is an output of a corresponding trained first stage model in the plurality of trained first stage models upon application of a respective patch in the plurality of patches to the corresponding trained first stage model; and
applying the feature vector to the second stage model thereby determining the cancer condition in the test subject.

35. The method of claim 34, wherein:
each respective trained first stage model in the plurality of trained first stage models is a corresponding trained convolutional neural network and the second stage model is a logistic regression model; and
the first channel of the first patch is two dimensional with each respective instance of the plurality of instances of the first plurality of parameters of the first patch forming a first dimension and the first plurality of parameters of the first patch forming the second dimension.

36. (canceled)

37. (canceled)

38. The method of claim 1, wherein:
the classifier comprises a plurality of first stage models and a dynamic neural network;
the at least one program further comprises instructions for constructing a plurality of patches including the first patch, each respective patch being for a different set of CpG sites in the reference genome;
the constructing B) constructs a respective patch including the first patch;
the applying at least the first patch to a classifier C) comprises:
C1) applying each respective patch in the plurality of patches to a corresponding first stage model in the plurality of first stage models, wherein the corresponding first stage model comprises:
i) a respective input layer for receiving the respective patch, wherein the respective patch comprises a first number of dimensions;
ii) a respective fully connected embedding layer that comprises a corresponding set of weights, wherein the respective fully connected embedding layer directly or indirectly receives output of the respective input layer, and wherein a respective output of the respective embedding layer is a second number of dimensions that is less than the first number of dimensions; and
iii) a respective output layer that directly or indirectly receives output from the respective fully connected embedding layer; and
C2) inputting an aggregate of the respective output from each respective fully connected embedding layer of each trained first stage model in the plurality of first stage models into the dynamic neural network thereby determining the cancer condition in the test subject.

39. (canceled)

40. The method of claim 38, wherein the at least one program further comprises instructions for training the plurality of first stage models and the dynamic neural network using a cohort of subjects, wherein the cohort of subjects comprises a first subset of subjects that have a first label for the cancer condition and a second subset of subjects that have a second label for the cancer condition.

41. (canceled)

42. The method of claim 40, wherein the cancer condition is tissue of origin and each subject in the cohort of subjects is labeled with a tissue of origin, and wherein the cohort includes subjects that have an anorectal cancer, a bladder cancer, a breast cancer, a cervical cancer, a colorectal cancer, a head and neck cancer, a hepatobiliary cancer, an endometrial cancer, a kidney cancer, a leukemia, a liver cancer, a lung cancer, a lymphoid neoplasm, a melanoma, a multiple myeloma, a myeloid neoplasm, an ovary cancer, a non-Hodgkin lymphoma, a pancreatic cancer, a prostate cancer, a renal cancer, a thyroid cancer, an upper gastrointestinal tract cancer, a urothelial carcinoma, or a uterine cancer.

43. (canceled)

44. The method of claim 40, wherein the cancer condition is a stage of a specified cancer and each subject in the cohort of subjects is labeled with a stage of a specified cancer, and wherein the cohort includes subjects that have a stage of an anorectal cancer, a stage of bladder cancer, a stage of breast cancer, a stage of cervical cancer, a stage of colorectal cancer, a stage of head and neck cancer, a stage of hepatobiliary cancer, a stage of endometrial cancer, a stage of kidney cancer, a stage of leukemia, a stage of liver cancer, a stage of lung cancer, a stage of lymphoid neoplasm, a stage of melanoma, a stage of multiple myeloma, a stage of myeloid neoplasm, a stage of ovary cancer, a stage of non-Hodgkin lymphoma, a stage of pancreatic cancer, a stage of prostate cancer, a stage of renal cancer, a stage of thyroid cancer, a stage of upper gastrointestinal tract cancer, a stage of urothelial carcinoma, or a stage of uterine cancer.

45. (canceled)

46. (canceled)

47. (canceled)

48. (canceled)

49. (canceled)

50. (canceled)

51. The method of claim 1, wherein the at least one program further comprises instructions for selecting the first independent set of CpG sites of the first patch through evaluation of a plurality of CpG methylation patterns determined by a methylation sequencing of a plurality of clinical fragments obtained from a plurality of clinical nucleic acid samples of a plurality of clinical biological samples obtained from a clinical cohort comprising a plurality of clinical subjects, wherein the plurality of clinical subjects includes a first set of clinical subjects that have a first indication for the cancer condition and a second set of clinical subjects that have a second indication for the cancer condition.

52. The method of claim 51, wherein the instructions for selecting comprise:
determining a first ranking of a plurality of CpG sites in the reference genome based upon a respective first mutual information score for a methylation status of each CpG site in the plurality of CpG sites between the first set of clinical subjects and the second set of clinical subjects; and
selecting a first threshold number of CpG sites for the corresponding independent set of CpG sites for the first patch using the ranking.

53. The method of claim 51, wherein:
the plurality of clinical subjects includes a third set of clinical subjects that have a third indication for the cancer condition and a fourth set of clinical subjects that have a fourth indication for the cancer condition and the instructions for selecting further comprise:
determining a second ranking of the plurality of CpG sites in the reference genome based upon a respective second mutual information score for a methylation status of each CpG site in the plurality of CpG sites between the third set of clinical subjects and the fourth set of clinical subjects; and
selecting a second threshold number of CpG sites for the first independent set of CpG sites of the first patch using the second ranking.

54. (canceled)

55. The method of claim 51, wherein the first indication for the cancer condition is a first cancer type and the second indication for the cancer condition is a second cancer type.

56.-82. (canceled)
